(19) United States
(12) Patent Application Publication
(10) Pub. No.: US 2025/0114465 A1
(43) Pub. Date: Apr. 10, 2025

(54) RNA DEGRADERS AND USES THEREOF

(71) Applicant: Arrakis Therapeutics, Inc., Waltham, MA (US)

(72) Inventors: Simon Aoyama, Waltham, MA (US); Bhalchandra Rao, Waltham, MA (US); Kathleen McGinness, Waltham, MA (US)

(21) Appl. No.: 18/705,376

(22) PCT Filed: Oct. 28, 2022
(86) PCT No.: PCT/US2022/078892

§ 371 Date: Apr. 26, 2024

Related U.S. Application Data
(60) Provisional application No. 63/263,208, filed on Oct. 28, 2021.

(51) Int. Cl. A61K 47/64

(52) U.S. Cl. CPC A61K 47/646

(57) ABSTRACT

The present invention includes compounds and compositions, and methods of use thereof for modulating an RNA transcript, or a precursor, isoform, fragment, or mutant thereof by degradation of the RNA transcript via recruitment or binding of one or more decay factors (e.g. an RNA binding protein). The invention also provides methods of preparing such compounds and compositions and methods of treating an RNA-mediated disease, disorder, or condition, comprising administering an effective amount of a disclosed compound or composition to a patient in need thereof. The present invention further provides compositions comprising an RNA binder and a ligand of an RNA-binding protein (RBP), as well as methods of use thereof, including for modulating the activity of an RNA transcript or a precursor, isoform, fragment, or mutant thereof, and treating diseases associated with the same.

### TECHNICAL FIELD OF THE INVENTION

The present invention relates to compounds and methods of preparation and use thereof for modulating the activity of RNA transcripts, as well as isoforms, mutants, and fragments thereof, via modulating their degradation and/or otherwise modulating their activity. The invention also provides methods of treating various diseases and conditions mediated by a target RNA transcript, such as those described herein.

### CROSS-REFERENCE TO RELATED APPLICATIONS

This application claims the benefit of U.S. Provisional Patent Application No. 63/263,208, filed on Oct. 28, 2021; the entire contents of which are hereby incorporated by reference.

### BACKGROUND OF THE INVENTION

RNA, both coding or messenger RNA (mRNA), as well as non-coding RNA (ncRNA), play a multitude of critical regulatory roles in the cell. The total of all RNAs transcribed from DNA-both coding and non-coding-comprise the transcriptome and all cellular biology flows from the transcriptome. All endogenous mammalian diseases are ultimately derived from or modulated by the transcriptome, either directly by RNA or through expressed proteins. Thus, there is the potential to intervene in all human diseases that are protein-mediated or RNA-mediated by modulating the translation or regulatory function of the corresponding mRNAs or ncRNAs.

RNA quality control (QC) mechanisms are varied and ubiquitous. After transcription, RNAs must undergo processing to produce their active forms. RNA processing includes a variety of endo- and exonucleolytic cleavage of sequences at either end of the initial transcript, cleavage of internal sequences (e.g., internal transcribed spacers and introns), nucleotide editing, and various types of functionalization via chemical modification. Notably, most cellular RNAs undergo multiple processing reactions, with alternate pathways (e.g., alternative splicing) leading to distinct products. Multiple RNAs from otherwise similar or identical RNA primary transcripts result in an increase in the functional diversity of RNA and protein species encoded by individual genes.

mRNA decay is the process that causes programmed nucleolytic degradation of the mRNA. The process is enabled by the association of mRNAs with specific RNA-binding proteins (RBPs). Thus, mRNA decay has the potential to directly influence the steady state levels of a translatable pool of mRNAs in vivo. Eukaryotic mRNA decay occurs primarily by enzymatic removal of nucleotides in the 5′-3′ direction and is catalyzed by Xrn1. mRNAs are also degraded in the 3′-5′ direction by the multi-subunit protein complex called the exosome, the catalytic subunit of which is Rrp44. The contribution of 3′-5′ decay to global mRNA turnover is higher in metazoans as compared to lower eukaryotes.

RNA QC mechanisms normally operate to eliminate incorrectly or incompletely processed RNAs. However, if the normal activity of these nucleases and QC pathways could be harnessed to selectively degrade (or not degrade) a disease-causing (or disease-treating) RNA target, it would lead to novel and indeed transformative modes of treating a variety of diseases.

Thus, there is a broad need for agents that selectively inhibit or eradicate target RNAs. The present invention achieves this using bifunctional or chimeric molecules and compositions that both (i) bind to target RNA transcripts and (ii) recruit decay factors, such as RNA-binding proteins (RBPs), that activate an RNA degradation mechanism to degrade the target RNAs or otherwise abrogate the function of the target RNAs (e.g., the availability of the RNA for translation into an active protein). The compounds of this invention and pharmaceutically acceptable compositions thereof meet these requirements and provide other related benefits, as described herein.

### SUMMARY OF THE INVENTION

An approach to modulating RNA function, as described herein, is to co-opt elements of the endogenous RNA quality control (QC) apparatus. As provided herein, it is possible to use compounds and compositions that include ligands that recruit endogenous nucleic-acid-modulating proteins, which, when recruited, modulate, inhibit or eradicate the RNA or its function. For example, RNA levels are frequently regulated by endonucleases or exonucleases in the cell. There are ligands which, when tethered to an RNA-targeted small molecule (rSM) or an RNA-targeted oligonucleotide, will bring endonucleases or exonucleases into proximity with the targeted RNA thereby accelerating degradation of the RNA and thus preventing its translation. We have surprisingly found that certain RNA-binding proteins (RBPs), such as CNOT2, CNOT7 and YTHDF2, at endogenous levels, can degrade target nucleic acids. The RBPs do not need to be induced or dimerize in order to act on the target RNA.

In one aspect, we describe chimeric molecules depicted generically below, comprising three moieties: (1) An RNA-binding small molecule (rSM) that binds to a targeted RNA and confers selectivity by directing a decay factor, e.g., an RBP, such as a nuclease, specifically to that RNA; (2) A decay factor-recruiting ligand, e.g., a small-molecule ligand that binds to the decay factor or associated protein(s) but leaves intact its activity (e.g. its nuclease activity; note that “nuclease” in this context encompasses endonucleases and exonucleases and the wide variety of nucleic-acid-modifying proteins described in more detail below); and (3) A linker that completes the chimera and brings the decay factor (e.g., the nucleic-acid-modifying proteins) into proximity with the RNA bound by the rSM, thereby allowing the decay factor (e.g., a nucleic-acid modifying protein) to specifically act on the target RNA. Together these elements comprise “NUTACs”: Nucleic Acid TArgeted Chimeras.

More specifically, in one aspect, the present invention provides a bifunctional compound of Formula A:

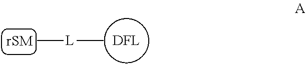

or a pharmaceutically acceptable salt thereof, wherein:
rSM is an RNA-binding small molecule that binds to a target RNA transcript;
DFL is a Decay Factor-recruiting Ligand; and
L is a bivalent linker group that covalently connects the rSM to the DFL;
wherein the DFL binds to or recruits one or more decay factors that degrade the target RNA transcript.

In some embodiments, the DFL is a small molecule that binds to an RNA-binding protein (RBP), and wherein binding of the DFL to the RBP modulates the target RNA transcript.

In some embodiments, the DFL recruits a nuclease by binding to the nuclease or by binding to a protein associated with or which recruits the nuclease, thus bringing the nuclease into proximity to the target RNA.

In some embodiments, the DFL recruits an RBP that is part of a multi-component complex that has nuclease activity by binding to the nuclease or by binding to a protein of the multi-component complex, thus bringing the nuclease into proximity to the target RNA. In some embodiments, the multi-component complex is a protein complex, a protein-nucleic acid complex or a protein-metabolite complex.

In some embodiments, the RBP that is being recruited is an RBP that at endogenous levels can modulate (e.g., degrade) the target RNA transcript. Thus, in some embodiments, no additional action needs to be taken (e.g., dimerizing and/or inducing the RBP) to allow for the RBP to act on the target RNA transcript.

In one aspect, the present invention provides a bifunctional compound that effects recruitment of a target RNA transcript (also referred to herein as an “RNA transcript,” “target RNA,” or “target transcript”) to a nuclease capable of degrading the target RNA, or to an RNA-binding protein (RBP) that destabilizes the target RNA transcript towards degradation by any of a cell's or tissue's endogenous mechanisms of RNA degradation.

The present invention further provides methods of preparing the disclosed bifunctional compounds and methods of use thereof in treating a disease, disorder, or condition.

In some embodiments, the present invention provides a bifunctional compound useful as a modulator of targeted degradation of a variety of target RNA transcripts, which are then degraded and/or otherwise inhibited by the bifunctional compounds as described herein. An advantage of the compounds provided herein is that a broad range of pharmacological activities is possible, consistent with the degradation/inhibition of a target RNA transcript from virtually any RNA class or family.

In some embodiments, the present invention provides a bifunctional composition comprising an RNA binder and a DFL. In some embodiments, the RNA binder is an oligonucleotide. In some embodiments, the RNA binder is an oligonucleotide, peptide, or oligosaccharide. In some embodiments, the DFL binds an RBP. In some embodiments, the present invention provides a bifunctional composition comprising an RNA binder and a DFL useful as a modulator of targeted degradation of a variety of target RNA transcripts, which are then degraded and/or otherwise inhibited by the bifunctional composition as described herein. An advantage of the composition provided herein is that a broad range of pharmacological activities is possible, consistent with the degradation/inhibition of a target RNA transcript from virtually any RNA class or family.

In addition, the present invention provides methods of using an effective amount of the compounds and compositions as described herein for the treatment or amelioration of a disease, disorder, or condition, such as those described herein.

In another aspect, the present invention provides a method of modulating the activity (e.g., the availability for protein translation) of a target RNA transcript or an isoform, fragment, or mutant thereof, comprising contacting the RNA transcript or an isoform, fragment, or mutant thereof with a disclosed compound or a pharmaceutically acceptable salt thereof that binds to the RNA transcript or an isoform, fragment, or mutant thereof.

In another aspect, the present invention provides a method of selectively degrading a target RNA transcript or an isoform, fragment, or mutant thereof, comprising contacting the RNA transcript or an isoform, fragment, or mutant thereof with a disclosed compound or a pharmaceutically acceptable salt thereof that binds to the RNA transcript or an isoform, fragment, or mutant thereof.

In another aspect, the present invention provides a method of identifying a bifunctional compound that binds to a target RNA transcript or an isoform, fragment, or mutant thereof, comprising i) contacting the target RNA transcript or an isoform, fragment, or mutant thereof with a disclosed compound and ii) analyzing the results by an assay disclosed herein, optionally in combination with a computational method and optionally comprising measuring degradation of the target RNA transcript or an isoform, fragment, or mutant thereof.

In one aspect the disclosure provides a compound of Formula A:

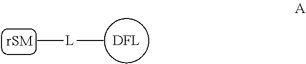

or a pharmaceutically acceptable salt thereof, wherein: rSM is an RNA-binding small molecule that binds to a target RNA transcript; DFL is a Decay Factor-recruiting Ligand; and
L is a bivalent linker group that covalently connects the rSM to the DFL; wherein the DFL binds to or recruits a decay factor. In some embodiments of the compounds provided herein, the decay factor is an RNA-binding protein (RBP) and binding of the DFL to the RBP leads to modulation of the target RNA transcript in vivo. In some embodiments of the compounds provided herein, modulation of the target RNA transcript in vivo is degradation of the target RNA transcript. In some embodiments of the compounds provided herein, the DFL binds the RBP without abrogating the enzymatic activity of the RBP and/or the ability of the RBP to be part of a multi-component complex, such as a protein complex. In some embodiments of the compounds provided herein, the RBP is an endonuclease, an exonuclease, a deadenylase, or a decapping protein, or the RBP is part of a multi-component complex that has endonuclease, exonuclease, deadenylase, or decapping activity. In some embodiments, the RBP destabilizes the target RNA transcript's 3-dimensional structure in a manner that makes it more prone to degradation. In some embodiments of the compounds provided herein, the RBP is one of those listed in Table 1B. In some embodiments of the compounds provided herein, the RBP has enzymatic activity, or is part of a multi-component complex that has enzymatic activity, at endogenous levels in vivo sufficient to measurably modulate the target RNA transcript or destabilize its 3-dimensional structure in a manner that makes it more prone to degradation. In some embodiments, the multi-component complex is a protein complex, a protein-nucleic acid complex or a protein-metabolite complex.

In some embodiments of the compounds provided herein, modulation of the target RNA transcript is degradation of the target RNA transcript. In some embodiments of the compounds provided herein, the RBP does not need to be induced to be active. In some embodiments of the compounds provided herein, the RBP does not need to dimerize to be active. In some embodiments of the compounds provided herein, the RBP is part of the CCR4-NOT (Carbon Catabolite Repression-Negative On TATA-less) complex. In some embodiments of the compounds provided herein, the RBP is CNOT2, CNOT7, DDX6, YTHDF2, ZFP36, DCP1A, ZC3H12A (Regnase-1), PARN, MARF, or IRE-1. In some embodiments of the compounds provided herein, the RBP is CNOT2. In some embodiments of the compounds provided herein, the RBP is CNOT7. In some embodiments of the compounds provided herein, the RBP is YTHDF2. In some embodiments of the compounds provided herein, the RBP is not RNase L. In some embodiments of the compounds provided herein, the DFL is one of those depicted in Table 1C. In some embodiments of the compounds provided herein, the target RNA transcript is an mRNA or a precursor, isoform, unspliced isoform, splicing intermediate, fragment, or mutant thereof. In some embodiments of the compounds provided herein, the target RNA transcript is selected from one of those listed in Table A, Table B, Table C, or Table D; or a precursor, isoform, unspliced isoform, splicing intermediate, fragment, or mutant thereof.

In some embodiments of the compounds provided herein, L is a covalent bond or a bivalent, saturated or unsaturated, straight or branched, optionally substituted C₁₋₅₀ hydrocarbon chain, wherein 0, 1, 2, 3, 4, 5, 6, 7, 8, 9, or 10 methylene units of L are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(NR₂)—, an amino acid, wherein:

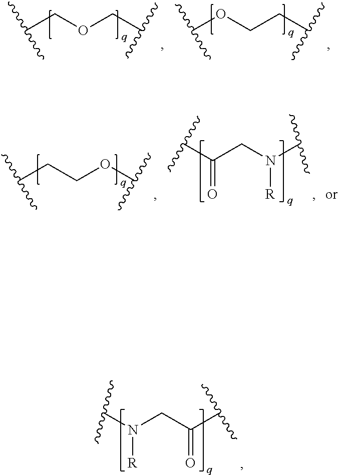

each -Cy²- is independently an optionally substituted bivalent ring selected from phenylene, an 8-12 membered bicyclic arylene, a 3-8 membered saturated or partially unsaturated carbocyclylene, an 8-12 membered bicyclic saturated or partially unsaturated carbocyclylene, a 3-8 membered saturated or partially unsaturated heterocyclylene having 1-3 heteroatoms independently selected from nitrogen, oxygen, and sulfur, an 8-12 membered bicyclic saturated or partially unsaturated heterocyclylene having 1-5 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur; and each q is independently 1, 2, or 3. In some embodiments of the compounds provided herein, L is selected from one of those depicted in Table 2.

In some embodiments of the compounds provided herein, the rSM is selected from any one of those described in the section entitled exemplary rSMs. In some embodiments of the compounds provided herein, the rSM is one of those shown in Table 1A.

In one aspect, the disclosure provides a composition comprising an RNA binder that binds to a target RNA transcript and a Decay Factor-recruiting Ligand (DFL), wherein the DFL binds to or recruits a decay factor. In some embodiments of the compositions provided herein, the decay factor is an RNA-binding protein (RBP) and binding of the DFL to the RBP leads to modulation of the target RNA transcript in vivo. In some embodiments of the compositions provided herein, modulation of the target RNA transcript in vivo is degradation of the target RNA transcript. In some embodiments of the compositions provided herein, the RNA binder is an oligonucleotide. In some embodiments of the compositions provided herein, the DFL binds the RBP without abrogating the enzymatic activity of the RBP and/or the ability of the RBP to be part of a multi-component complex, such as a protein complex. In some embodiments of the compositions provided herein, the RBP is an endonuclease, an exonuclease, a deadenylase, or a decapping protein, or the RBP is part of a multi-component complex that has endonuclease, exonuclease, deadenylase, or decapping activity. In some embodiments, the RBP destabilizes the target RNA transcript's 3-dimensional structure in a manner that makes it more prone to degradation. In some embodiments of the compositions provided herein, the RBP is one of those listed in Table 1B. In some embodiments of the compositions provided herein, the RBP has enzymatic activity, or is part of a multi-component complex that has enzymatic activity, at endogenous levels in vivo sufficient to measurably modulate the target RNA transcript or destabilize its 3-dimensional structure in a manner that makes it more prone to degradation. In some embodiments, the multi-component complex is a protein complex, a protein-nucleic acid complex or a protein-metabolite complex.

In some embodiments of the compositions provided herein, the modulation of the target RNA transcript is degradation of the target RNA transcript. In some embodiments of the compositions provided herein, the RBP does not need to be induced to be active. In some embodiments of the compositions provided herein, the RBP does not need to dimerize to be active. In some embodiments of the compositions provided herein, the RBP is part of the CCR4-NOT (Carbon Catabolite Repression-Negative On TATA-less) complex. In some embodiments of the compositions provided herein, the RBP is CNOT2, CNOT7, DDX6, YTHDF2, ZFP36, DCP1A, ZC3H12A (Regnase-1), PARN, MARF, or IRE-1. In some embodiments of the compositions provided herein, the RBP is CNOT2. In some embodiments of the compositions provided herein, the RBP is CNOT7. In some embodiments of the compositions provided herein, the RBP is YTHDF2. In some embodiments of the compounds provided herein, the RBP is not RNase L. In some embodiments of the compositions provided herein, the DFL is one of those depicted in Table 1C. In some embodiments of the compositions provided herein, the target RNA transcript is an mRNA or a precursor, isoform, unspliced isoform, splicing intermediate, fragment, or mutant thereof. In some embodiments of the compositions provided herein, the target RNA transcript is selected from one of those listed in Table A, Table B, Table C, or Table D; or a precursor, isoform, unspliced isoform, splicing intermediate, fragment, or mutant thereof.

In one aspect, the disclosure provides a pharmaceutical composition comprising any one of the compounds provided herein, or a pharmaceutically acceptable salt thereof, or any one of the compositions provided herein; and a pharmaceutically acceptable carrier.

In one aspect, the disclosure provides a method of modifying the amount of a protein in a cell, the method comprising administering a compound or composition that acts on a target RNA transcript or a precursor, isoform, fragment, or mutant thereof, in an amount sufficient to modify the amount of the protein in the cell. In some embodiments of the methods provided herein, modifying the amount of a protein in a cell is reducing the amount of protein in the cell. In some embodiments of the methods provided herein, the method comprises administering any one of the compounds provided herein, or a pharmaceutically acceptable salt thereof, or any one of the compositions provided herein; and a pharmaceutically acceptable carrier.

In one aspect, the disclosure provides a method of modulating the availability for protein translation of a target RNA transcript or a precursor, isoform, fragment, or mutant thereof, comprising contacting the target RNA transcript or a precursor, isoform, fragment, or mutant thereof with any one of the compounds provided herein, or a pharmaceutically acceptable salt thereof, or any one of the compositions provided herein; and a pharmaceutically acceptable carrier.

In one aspect, the disclosure provides a method of modulating the translation of a target protein or mutant thereof, comprising contacting a target RNA transcript or a precursor, isoform, fragment, or mutant thereof with any one of the compounds provided herein, or a pharmaceutically acceptable salt thereof, or any one of the compositions provided herein; and a pharmaceutically acceptable carrier.

In one aspect, the disclosure provides a method of decreasing the half-life or increasing degradation of a target RNA transcript or a precursor, isoform, fragment, or mutant thereof, comprising contacting the target RNA transcript or the precursor, isoform, fragment, or mutant thereof with any one of the compounds provided herein, or a pharmaceutically acceptable salt thereof, or any one of the compositions provided herein; and a pharmaceutically acceptable carrier.

In one aspect, the disclosure provides a method of treating a disease, comprising administering to a subject in need thereof any one of the compounds provided herein, or a pharmaceutically acceptable salt thereof, or any one of the compositions provided herein; and a pharmaceutically acceptable carrier. In some embodiments of the methods provided herein, the disease is characterized by an aberrant level of a protein in a cell. In some embodiments of the methods provided herein, the disease is one of those listed in Table A, Table B, Table C, or Table D. In some embodiments of the methods provided herein, the disease is a cancer. In some embodiments of the methods provided herein, the method induces proximity of an RNA-binding protein (RBP) to the target RNA transcript, and the RBP is CNOT2, CNOT7, DDX6, YTHDF2, ZFP36, DCP1A, ZC3H12A (Regnase-1), PARN, MARF, or IRE-1; and the target RNA transcript is a pre-mRNA, mature mRNA, or partially processed mRNA, or an isoform, fragment, or mutant thereof.

In one aspect, the disclosure provides a method of inducing degradation or decreasing the half-life of a target RNA transcript or an isoform, fragment, or mutant thereof, comprising contacting the target RNA transcript or the isoform, fragment, or mutant thereof with an RNA-binding moiety that induces proximity of an RNA-binding protein (RBP). In some embodiments of the methods provided herein, the RBP is one of those listed in Table 1B. In some embodiments of the methods provided herein, the RBP is CNOT2, CNOT7, DDX6, YTHDF2, ZFP36, DCP1A, ZC3H12A (Regnase-1), PARN, MARF, or IRE-1. In some embodiments of the methods provided herein, the RBP is CNOT2, CNOT7, or YTHDF2.

### BRIEF DESCRIPTION OF THE FIGURES

FIG. 1 shows an overview of a genetic tethering assay to determine RNA degradation activity of RBPs at endogenous activity levels.

FIG. 2A and FIG. 2B depict bar graphs of genetic tethering experiments that show that RNA-binding proteins (RBPs) capable of degrading a target RNA can do so in a reporting system with promoters that express reporters and RBPs at endogenous (physiological) levels. The genetic tethering assay identified multiple RNA-binding proteins (RBPs) with consistent activity at endogenous levels. Various reporters and readouts are shown for CNOT2, CNOT7 and ZFP36 in FIG. 2A, while FIG. 2B shows data for CNOT2, CNOT7, YTHDF2, DDX6, DCP1A, ZFP36 and PARN with an endogenous level promoter. NCI-H1299 cells (ATCC), which provide a physiologically relevant model, were engineered to stably express various λN-tagged RBPs via lentivirus. Cells were then transfected using Lipofectamine 3000 (Thermo Fisher) with two plasmids: one encoding a reporter luciferase containing a BoxB site in either the 3′ untranslated region (UTR) or the 5′ UTR and one normalizer plasmid encoding an alternative luciferase without any BoxB sites for tethering. After 24 hours, expression of both luciferases wes measured using commercially available kits (Promega). Data are expressed as a ratio of reporter expression (measured in relative light units or RLUs) to normalizer expression.

### DETAILED DESCRIPTION OF CERTAIN EMBODIMENTS

### 1. General Description of Certain Embodiments of the Invention; Definitions

Targeting RNA Transcripts with Compounds of the Present Invention

It is known that RNA interference (RNAi) complexes recruit ribonuclease to cleave the bound, targeted RNA. See, for example, Velagapudi, S. P., et al., “Design of a small molecule against an oncogenic noncoding RNA,” Proc Natl Acad Sci USA 2016 113 (21), 5898-903; Disney, M. D., “Targeting RNA with Small Molecules To Capture Opportunities at the Intersection of Chemistry, Biology, and Medicine,” J. Am. Chem. Soc. 2019 141 (17), 6776-6790; Ursu, A. et al., “Methods to identify and optimize small molecules interacting with RNA (SMIRNAs),” Drug Discovery Today 2019 October; 24 (10): 2002-2016; each of which is hereby incorporated by reference. And of course, in proteins, there is a burgeoning industry around protease-targeted chimeras (ProTacs) comprised of ligands that bind to the targeted proteins and (typically) ligands that recruit the E3-ligase apparatus which, by virtue of linker-imposed proximity, ubiquitinylates the target protein, and thereby labels the target protein for degradation by the proteasome system.

Distinct molecular features protect mRNA from constitutive degradation and are essential for the stability of the mRNA. Mature eukaryotic mRNAs contain a 5′-cap, which consists of a 5′-methylguanosine residue linked to the mRNA in a 5′-5′ linkage. The 5′-cap is resistant to the activity of Xrn1 and protects the mRNA from 5′-3′ mRNA degradation. The 3′-end of the mRNA is protected by a series of adenosine residues, also referred to as a polyA tail. The poly A residues are bound by the polyA binding protein (PABP), which restricts the access of the exosome to the 3′-end. Deadenylation, which is the successive removal of As from the polyA tail, occurs over the lifespan of the mRNA. The resultant short polyA tail is no longer protected by PABP and instead the mRNA gets exposed to 3′-5′ degradation.

The initiation of mRNA degradation is highly choreographed. Most mRNAs are turned over via a process called “deadenylation-dependent decay.” A deadenylated (short polyA-tailed) mRNA recruits translational repressor and decapping enhancer proteins, which in turn recruit the Dcp1-2 enzymes that cause mRNA decapping. Thus, deadenylation can affect the decay rates of most mRNA in vivo. This is corroborated by the finding that artificial tethering of deadenylation proteins to mRNA significantly reduces their half-lives. In some instances, mRNAs contain distinct cis-elements that promote their degradation as part of an autoregulatory mechanism. Such cis-elements, for instance AU-rich elements present in mRNA 3′-ends, cause binding of regulatory RBPs that promote deadenylation-dependent decay by recruiting deadenylation factors to the mRNA. Lastly, in some exceptional cases, decapping occurs independent of deadenylation. Such mechanisms are a part of specialized mRNA decay mechanisms.

In addition to normal mRNA turnover pathways, distinct quality control mechanisms have evolved that surveil mRNA to ensure high-fidelity gene expression. mRNA surveillance systems ensure that mRNA, which may contain deleterious mutations that can cause faulty protein production, are identified and degraded at rates that are significantly faster than their natural turnover rates. Distinct mRNA surveillance mechanisms identify specific types of aberrant mRNA, such as mRNA with pre-termination codons, mRNA that lack natural stop codons, and mRNA that contain mutations that lead to extended ribosomal pausing on an mRNA. Specialized mRNA decay mechanisms, such as Nonsense mediated decay (NMD), Non-stop decay, and No-go decay enable enhanced degradation of the above listed aberrant mRNA, respectively.

Whether at the end of an mRNA natural life cycle or via quality control pathways, the RNA-binding proteins that promote the degradation of mRNA can be broadly classified as “mRNA-destabilizing proteins.” Once stably associated with mRNAs, most mRNA-destabilizing proteins recruit deadenylation and/or decapping proteins to target mRNA for degradation. The association of such mRNA destabilizing proteins to mRNA is often epistatic to other upstream RBP interactions and penultimate to degradation. In some instances, mRNA destabilization proteins also function by recruiting select mRNA endonucleases (see below) to degrade mRNA.

Lastly, while the majority of mRNA decay is initiated by 5′-3′ and 3′-5′ exononucleolytic mechanisms, endonucleolytic cleavage of target mRNA is observed under some special circumstances. Select endonucleases have been identified, such as SMG6, Cue2, RNase L, and IRE1, which cleave mRNAs internally as a part of responses such as NMD, No-go decay, viral defense, unfolded protein response (UPR), respectively. In a few notable instances, endonucleolytic cleavage has been documented as a part of the natural turnover of mRNA, such as c-myc, c-fos, MITF etc.

The vast majority of molecular targets that have been addressed therapeutically are proteins. However, it is now understood that RNA plays important regulatory roles in both healthy and diseased cells. A bifunctional compound that binds to a target RNA transcript such as an mRNA transcript or mature mRNA can modulate the activity of the RNA transcript (e.g., its availability to be translated into a functional protein) by increasing or decreasing its in vivo half-life, e.g., by increasing or decreasing the rate of degradation of the RNA. In some embodiments, the rate of degradation is modulated via recruitment or activation of an RNA-degrading mechanism such as a nuclease and/or RNA-destabilizing protein. Such recruitment and/or activation occurs by binding of a portion of the bifunctional compound to an RBP or other protein involved in an RNA degradation process. For RNAs that encode proteins, this process will thus affect expression levels of the encoded protein.

Genetic tethering assays are routinely employed to elucidate a role for an RBP in mRNA destabilization and mRNA decay. Genetic tethering involves artificial association of RBPs with reporter mRNA using viral-derived high affinity RNA-protein interactions e.g., MS2 RNA-MS2 protein and BoxB-AN. See, for example, Luo, E.-C. et al., Nature Structural & Molecular Biology volume 27, pages 989-1000 (2020), which is hereby incorporated by reference. However, it should be appreciated that most genetic tethering assays are designed to artificially maximize the “signal” of the experiment. Provided herein are genetic tethering assays that allow for the identification of RBPs that are active at endogenous conditions.

In some embodiments, genetic tethering is employed to assay the association of one or more mRNA destabilizing proteins with an mRNA target to induce its degradation. In some embodiments, genetic tethering is employed to assay the association of one or more mRNA destabilizing proteins with an mRNA target to induce its degradation, wherein the one or more mRNA destabilizing proteins are present at endogenous levels.

In one aspect, the present invention provides an mRNA destabilizing protein, such as a ribonuclease, tethered to a small molecule that binds an mRNA target.

In one aspect, the present disclosure provides a bifunctional compound or composition that effects recruitment of a decay factor to a target RNA. In some embodiments the decay factor is an mRNA destabilizing protein, a nuclease and/or an RNA-binding protein. However, decay factors are not so limited and include any protein that interferes with the stability and/or activity of the RNA. It should further be appreciated that some RNA-binding proteins also have nuclease activity. In some embodiments, the present disclosure provides a bifunctional compound or composition that effects recruitment to a target RNA to a nuclease (e.g., an RBP) capable of degrading the target RNA, or to an RNA-binding protein (RBP) that destabilizes the target RNA towards degradation by any of a cell's or tissue's mechanisms of RNA degradation.

In one aspect, the present disclosure provides a bifunctional compound or composition that effects recruitment of a target RNA to a decay factor, wherein the decay factor is present at endogenous levels. In some embodiments, the present disclosure provides a bifunctional compound or composition that effects recruitment of a target RNA to a nuclease capable of degrading the target RNA, wherein the nuclease is present at endogenous levels, or to an RNA-binding protein (RBP) that destabilizes the target RNA towards degradation by any of a cell's or tissue's mechanisms of RNA degradation, wherein the RBP that destabilizes the target RNA is present at endogenous levels.

In some embodiments, the compounds or compositions disclosed herein bind to an mRNA transcript or mature mRNA. In some embodiments, the compounds include an rSM and the compound binds the target RNA through its rSM. Generally, the rSM will bind specifically to a particular target RNA, resulting in the selective degradation of the target RNA.

In some embodiments, the composition includes an RNA binder, such as an oligonucleotide, and the composition binds the RNA through its oligonucleotide. Oligonucleotides that bind RNA are well known. Generally, the oligonucleotide that binds the target RNA will have a nucleic acid sequence that is complementary to a nucleic acid sequence in the target RNA. The binding of an oligonucleotide with a complimentary sequence to a target RNA sequence is stable and highly specific. In some embodiments, the composition including an RNA binder, such as an oligonucleotide, is optimized for intracellular delivery. Optimization of oligonucleotides and compositions comprising oligonucleotides for intracellular delivery is well established.

In some embodiments, the composition comprises an RNA binder. In some embodiments, the RNA binder is an oligonucleotide. In some embodiments, the oligonucleotide can specifically bind an RNA target. In some embodiments, the oligonucleotide comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. In some embodiments, the oligonucleotide consists of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. In some embodiments, the oligonucleotide has been modified for therapeutic delivery.

In some embodiments, the target RNA transcript is a noncoding RNA such as microRNA (miRNA) or long noncoding RNA (lncRNA). In some embodiments, such RNA transcripts regulate transcription, splicing, mRNA stability/decay, or translation. In addition, the noncoding regions of mRNA such as the 5′ untranslated regions (5′ UTR), the 3′ UTR, and introns can play regulatory roles in affecting mRNA expression levels, alternative splicing, translational efficiency, and mRNA and protein subcellular localization. Without wishing to be bound by theory, it is believed that RNA secondary and tertiary structures are critical for these regulatory activities. Accordingly, modulation of the activity of a target RNA transcript or an isoform, fragment, or mutant thereof is possible by binding of a disclosed compound at one or more than one binding site.

In one aspect, the present disclosure provides a bifunctional compound of Formula A:

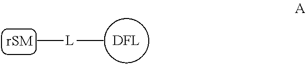

or a pharmaceutically acceptable salt thereof, wherein:
rSM is an RNA-binding small molecule that binds to a target RNA transcript;
DFL is a Degrading Factor-recruiting Ligand; and
L is a bivalent linker group that covalently connects the rSM to the DFL;
wherein the DFL binds to or recruits one or more decay factors that degrade the target RNA transcript.

In some embodiments, the DFL is a small molecule that binds to an RNA-binding protein (RBP), and wherein binding of the DFL to the RBP modulates the target RNA transcript (e.g., modulates the half-life of the target RNA transcript).

In some embodiments, the DFL binds to a protein that is associated with or which recruits a nuclease. In some embodiments, the DFL recruits a nuclease by binding to the nuclease or by binding to a protein associated with or which recruits the nuclease, thus bringing the nuclease into proximity with the target RNA.

In one aspect, the present invention provides a method of modulating the activity of a target RNA transcript or an isoform, fragment, or mutant thereof, comprising contacting the target RNA transcript or an isoform, fragment, or mutant thereof with a disclosed compound or a pharmaceutically acceptable salt thereof that binds to the target RNA transcript or an isoform, fragment, or mutant thereof.

In another aspect, the present invention provides a method of modulating the activity of a target protein or mutant thereof, comprising contacting a corresponding target RNA transcript or an isoform, fragment, or mutant thereof with a disclosed compound or a pharmaceutically acceptable salt thereof that binds to the target RNA transcript or an isoform, fragment, or mutant thereof.

In one aspect, the present invention provides a method of decreasing the half-life or increasing degradation of a target RNA transcript or an isoform, fragment, or mutant thereof, comprising contacting the target RNA transcript or an isoform, fragment, or mutant thereof with a disclosed compound that binds to the target RNA transcript or an isoform, fragment, or mutant thereof.

In some embodiments, translation of the target RNA transcript is decreased or inhibited, e.g., by decreasing the half-life of the transcript. In some embodiments, production of the corresponding functional protein or a mutant thereof is decreased or inhibited.

In some embodiments, the target RNA transcript is one which under normal physiological conditions, or in a disease state that is to be treated by a compound of the present invention, has a relatively long half-life in the body. In some embodiments, the half-life is at least 72 hours. In some embodiments, the half-life is at least 48, 24, 20, 18, 16, 14, 12, 10, 8, 6, 4, or 3 hours. In some embodiments, the half-life is about 24, 20, 18, 16, 14, 12, 10, 8, 6, 4, or 3 hours. In some embodiments, the half-life is about 3 to about 72 hours. In some embodiments, the half-life is about 4 to about 48 hours, or about 4-24, 4-18, 6-72, 6-48, 6-24, 6-18, 8-72, 8-48, 8-24, 8-18, 10-72, 10-48, 10-24, 10-18, 12-72, 12-48, 12-24, 12-18, 14-72, 14-48, 14-24, 14-18, 16-72, 16-48, 16-24, 16-18, 18-72, 18-48, 18-24, 20-72, 20-48, 20-24, 22-72, 22-48, 22-24, 24-72, 24-48, 30-72, or 30-48 hours.

In some embodiments, the administration of a compound or composition provided herein results in decrease or inhibition of the production of a functional protein or a mutant thereof. In some embodiments, the production of a functional protein or a mutant thereof is decreased by at least 10%, at least 20%, at least 30%, at least 40%, at least 25%, at least 60%, at least 70%, at least 80%, at least 90%, or is no longer produced at detectable levels.

In some embodiments, the activity of the target RNA transcript or an isoform, fragment, or mutant thereof is inhibited or decreased. In some embodiments, processing or splicing of the target RNA transcript or an isoform, fragment, or mutant thereof is inhibited.

In some embodiments, the target RNA is an mRNA, or a precursor, isoform, fragment, or mutant thereof. In some embodiments, inhibition of processing or splicing results in a decrease in levels of mature mRNA and/or protein. In some embodiments, the activity of the protein or mutant thereof is inhibited or decreased, e.g., due to a decreased level of the protein in a cell.

In some embodiments, the target RNA transcript comprises a functionally relevant fragment of a disease-causing RNA. A target RNA transcript or an isoform, fragment, or mutant thereof is “functionally relevant” if it includes at least a portion of a target RNA transcript that is ultimately transcribed and that is essential to producing a corresponding, disease-causing functional protein or mutant thereof.

In some embodiments, the target RNA transcript is a pre-mRNA, mature mRNA, or partially processed mRNA, or an isoform, fragment, or mutant thereof.

In some embodiments, the RNA transcript comprises a 5′ untranslated region (UTR).

In some embodiments, the RNA transcript comprises an open reading frame (ORF).

In some embodiments, the RNA transcript comprises a 5′ cap.

In some embodiments, the RNA transcript comprises a 3′ polyA tail (polyadenylated tail).

In some embodiments, the compound binds to a 5′ untranslated region (5′ UTR), a 3′ UTR, or an intron present in the RNA transcript.

In some embodiments, translation of the RNA transcript is reduced. In some embodiments, levels of protein encoded by the RNA transcript are decreased in a biological sample contacted with a disclosed compound or composition, such as a cell culture, or decreased in a patient treated with a disclosed compound or composition. In some embodiments, degradation of the RNA transcript is increased. In some embodiments, degradation of the RNA transcript is increased due to binding of the disclosed compound.

In one aspect, the present invention provides a method of identifying a compound that binds to a target RNA transcript or an isoform, fragment, or mutant thereof, comprising i) contacting the target RNA transcript or an isoform, fragment, or mutant thereof with a disclosed compound and ii) analyzing the results by an assay disclosed herein, optionally in combination with a computational method. In some embodiments, the method comprises the use of an SEC-MS, SPR, or DEL screen to identify the compound.

In another aspect, the present invention provides a method of treating an RNA-mediated disease, disorder, or condition (which includes any protein-mediated disease, disorder or condition) in a patient in need thereof, comprising administering to the patient an effective amount of a disclosed compound or a pharmaceutically acceptable salt thereof. In some embodiments, the disease, disorder, or condition is a proliferative disorder, such as a cancer.

A variety of RNA transcripts are appropriate as target RNA transcripts for use in the present invention. In some embodiments, the target RNA transcript is selected from one of those in Table A, Table B, Table C, or Table D below, or a precursor, isoform, unspliced isoform, splicing intermediate, fragment, or mutant thereof.

In some embodiments, the target RNA transcript is single-stranded. In some embodiments, the target RNA transcript is double-stranded or partially double-stranded. In some embodiments, the target RNA is a pair of nucleic acids engaged in an interaction, such as a miRNA-mRNA hybridized (or partially hybridized) pair. In some embodiments, the target RNA comprises one, two, or more miRNAs bound to an mRNA. In some embodiments, the target RNA is an mRNA, miRNA, premiRNA, or a viral or fungal RNA.

In some embodiments, the target RNA transcript includes structural features such as at least some intramolecular base pairing, a junction (e.g., cis or trans three-way junctions (3WJ)), quadruplex, hairpin, triplex, bulge loop, pseudoknot, or internal loop, etc., and any transient forms or structures adopted by the nucleic acid. In some embodiments, the target RNA transcript includes a bound protein, such as a chaperone, RNA-binding protein (RBP), or other nucleic acid-binding protein.

Target RNA transcripts of various lengths are target RNA transcripts within the scope of the present invention. For example, the target RNA may be from 20-10,000 nucleotides in length. In some embodiments, the target RNA is a relatively short sequence of, e.g., less than 250, less than 100, or less than 50 nucleotides in length. In some embodiments, the target RNA is 100 or more nucleotides in length. In some embodiments, the target RNA is 250 or more nucleotides in length. In some embodiments, the target RNA is up to about 350, 450, 500, 600, 750, or 1,000, 2,000, 3,000, 4,000, 5,000, 7,500, 10,000, 15,000, 25,000, 50,000, or more than 50,000 nucleotides in length. In some embodiments, the target RNA is between about 30 and about 500 nucleotides in length. In some embodiments, the target RNA is between about 250 and about 1,000 nucleotides in length. In some embodiments, the target RNA is between about 20-50, 30-60, 40-70, 50-80, 20-100, 30-100, 40-100, 50-100, 20-200, 30-200, 40-200, 50-200, 20-300, 50-300, 75-300, 100-300, 20-400, 50-400, 100-400, 200-400, 20-500, 50-500, 100-500, 250-500, 20-750, 50-750, 100-750, 250-750, 500-750, 20-1,000, 100-1,000, 250-1,000, 500-1,000, 20-2,000, 100-2,000, 500-2,000, 1,000-2,000, 20-5,000, 100-5,000, 1,000-5,000, 20-10,000, 100-10,000, 1,000-10,000, or 20-25,000 nucleotides in length.

Where the target or other referenced nucleic acid is an RNA, “nucleotides” refers to ribonucleotides. Where the target or other referenced nucleic acid is DNA, “nucleotides” refers to 2′-deoxyribonucleotides. In some embodiments, a target RNA comprises one or more nucleotide analogs (modified nucleotides) as defined herein and as known in the art.

In some embodiments, the target RNA is a pre-mRNA, pre-miRNA, pretranscript, partially spliced mRNA, fully spliced mRNA, fully spliced and partially processed mRNA, or a mature mRNA (i.e., fully spliced and processed mRNA).

In some embodiments, the RNA is a non-coding RNA (ncRNA), messenger RNA (mRNA), micro-RNA (miRNA), a ribozyme, riboswitch, lncRNA, lincRNA, snoRNA, snRNA, scaRNA, piRNA, rRNA, ceRNA, or pseudo-gene, wherein each of the foregoing may be selected from a human or non-human RNA, such as viral RNA, fungal RNA, or bacterial RNA.

Targeting mRNA

In some embodiments, the target RNA transcript is an mRNA or a precursor to a mature mRNA; or an isoform, fragment, or mutant thereof. Within mRNAs, noncoding regions can affect the level of mRNA and protein expression. Briefly, these include internal ribosome entry sites (IRES) and upstream open reading frames (uORF) that affect translation efficiency, intronic sequences that affect splicing efficiency and alternative splicing patterns, 3′ UTR sequences that affect mRNA and protein localization, and elements that control mRNA decay and half-life. Therapeutic modulation of these RNA elements can have beneficial effects. Also, mRNAs may contain expansions of simple repeat sequences such as trinucleotide repeats. These repeat expansion containing RNAs can be toxic and have been observed to drive disease pathology, particularly in certain neurological and musculoskeletal diseases (see Gatchel & Zoghbi, Nature Rev. Gen. 2005, 6, 743-755). Accordingly, in some embodiments, the present invention provides a method of degrading an mRNA that contains a toxic repeat expansion, or an isoform, fragment, or mutant thereof, comprising contacting the mRNA with a disclosed compound. The present invention further provides a method of treating a disease, disorder, or condition mediated by an mRNA that contains a toxic repeat expansion, or an isoform, fragment, or mutant thereof.

Additionally, in some embodiments, the expression of a target mRNA and its translation products is modulated by targeting noncoding sequences and structures in the 5′ and 3′ UTRs. For instance, RNA structures in the 5′ UTR can affect translational efficiency. RNA structures such as hairpins in the 5′ UTR have been shown to affect translation. In general, RNA structures are believed to play a critical role in translation of mRNA. One example of these are internal ribosome entry sites (IRES), which can affect the level of translation of the main open reading frame (Komar and Hatzoglou, Frontiers Oncol. 5:233, 2015; Weingarten-Gabbay et al., Science 351, 4939, 2016; Calvo et al., Proc. Natl. Acad. Sci. USA 106:7507-7512; Le Quesne et al., J. Pathol. 220:140-151, 2010; Barbosa et al., PLOS Genetics 9: e10035529, 2013). Small molecules targeting these RNAs could be used to modulate specific protein levels for therapeutic benefit. In some embodiments, the small molecule rSM binding site is a 5′ UTR, internal ribosome entry site, or upstream open reading frame.

### Non-Coding RNA Transcripts

Non-coding RNAs regulate cellular biology directly through function of RNA structures (e.g., ribonucleoproteins) as well as via regulating protein expression. These ncRNAs include (but are not limited to) miRNA, lncRNA, lincRNA, snoRNA, snRNA, scaRNA, piRNA, ceRNA, and pseudo-genes. Drugs that intervene at this level have the potential of modulating any cellular process.

In some embodiments, the target RNA transcript is an RNA that is transcribed but not translated into protein, termed “non-coding RNA” or “ncRNA.” Non-coding RNA is highly conserved, and the many varieties of non-coding RNA play a wide range of regulatory functions. The term “non-coding RNA,” as used herein, includes but is not limited to micro-RNA (miRNA), long non-coding RNA (lncRNA), long intergenic non-coding RNA (lincRNA), Piwi-interacting RNA (piRNA), competing endogenous RNA (ceRNA), and pseudo-genes. Each of these sub-categories of non-coding RNA offers a large number of RNA targets with significant therapeutic potential. Accordingly, in some embodiments, the present invention provides methods of treating a disease mediated by a non-coding transcript. In some embodiments, the disease is caused by a lncRNA, lincRNA, ceRNA, or pseudo-gene. In another aspect, the present invention provides a method of producing a small molecule that modulates the activity of a target non-coding transcript to treat a disease or disorder, comprising the steps of: screening one or more disclosed compounds for binding to or degradation of the target non-coding transcript; and analyzing the results by an RNA binding assay disclosed herein. In some embodiments, the target non-coding transcript is a lncRNA, lincRNA, ceRNA, or pseudo-gene.

In some embodiments, the target RNA transcript is an miRNA. miRNA are short double-strand RNAs that regulate gene expression (see Elliott & Ladomery, Molecular Biology of RNA, 2^{nd} Ed.). Each miRNA can affect the expression of many human genes. There are nearly 2,000 miRNAs in humans. These RNAs regulate many biological processes, including cell differentiation, cell fate, motility, survival, and function. miRNA expression levels vary between different tissues, cell types, and disease settings. They are frequently aberrantly expressed in tumors versus normal tissue, and their activity may play significant roles in cancer (for reviews, see Croce, Nature Rev. Genet. 10:704-714, 2009; Dykxhoorn Cancer Res. 70:6401-6406, 2010). miRNAs have been shown to regulate oncogenes and tumor suppressors and themselves can act as oncogenes or tumor suppressors. Some have been shown to promote epithelial-mesenchymal transition (EMT) and cancer cell invasiveness and metastasis. In the case of oncogenic miRNAs, their inhibition could be an effective anti-cancer treatment. Accordingly, in one aspect, the present invention provides a method of producing a small molecule that modulates the activity of a target miRNA to treat a disease or disorder, comprising the steps of: screening one or more disclosed compounds for binding to or degradation of the target miRNA; and analyzing the results by an RNA binding assay disclosed herein. In some embodiments, the miRNA regulates an oncogene or tumor suppressor, or acts as an oncogene or tumor suppressor. In some embodiments, the disease is cancer. In some embodiments, the cancer is a solid tumor.

Beyond oncology, miRNAs play roles in many other diseases including cardiovascular and metabolic diseases (Quiant and Olson, J. Clin. Invest. 123:11-18, 2013; Olson, Science Trans. Med. 6: 239ps3, 2014; Baffy, J. Clin. Med. 4:1977-1988, 2015).

Many mature miRNAs are relatively short in length and thus may lack sufficient folded, three-dimensional structure to be targeted by small molecules. However, it is believed that the levels of such miRNA could be reduced by small molecules that bind the primary transcript or the pre-miRNA to block the biogenesis of the mature miRNA. Accordingly, in some embodiments of the methods described above, the target miRNA is a primary transcript or pre-miRNA whose corresponding mature miRNA affects an oncogene or tumor suppressor, or which affects the levels or activity of a disease-causing RNA transcript or protein.

In some embodiments, the target RNA transcript is an lncRNA. lncRNA are RNAs of over 200 nucleotides (nt) that do not encode proteins (see Rinn & Chang, Ann. Rev. Biochem. 2012, 81, 145-166; (for reviews, see Morris and Mattick, Nature Reviews Genetics 15:423-437, 2014; Mattick and Rinn, Nature Structural & Mol. Biol. 22:5-7, 2015; Iyer et al., Nature Genetics 47 (: 199-208, 2015)). They can affect the expression of the protein-encoding mRNAs at the level of transcription, splicing and mRNA decay. Considerable research has shown that lncRNA can regulate transcription by recruiting epigenetic regulators that increase or decrease transcription by altering chromatin structure (e.g., Holoch and Moazed, Nature Reviews Genetics 16:71-84, 2015). lncRNAs are associated with human diseases including cancer, inflammatory diseases, neurological diseases and cardiovascular disease (for instance, Presner and Chinnaiyan, Cancer Discovery 1:391-407, 2011; Johnson, Neurobiology of Disease 46:245-254, 2012; Gutscher and Diederichs, RNA Biology 9:703-719, 2012; Kumar et al., PLOS Genetics 9: e1003201, 2013; van de Vondervoort et al., Frontiers in Molecular Neuroscience, 2013; Li et al., Int. J. Mol. Sci. 14:18790-18808, 2013). In general, lncRNA are expressed at a lower level relative to mRNAs. Many lncRNAs are physically associated with chromatin (Werner et al., Cell Reports 12, 1-10, 2015) and are transcribed in close proximity to protein-encoding genes. They often remain physically associated at their site of transcription and act locally, in cis, to regulate the expression of a neighboring mRNA.

lncRNAs regulate the expression of protein-encoding genes, acting at multiple different levels to affect transcription, alternative splicing and mRNA decay. For example, lncRNA has been shown to bind to the epigenetic regulator PRC2 to promote its recruitment to genes whose transcription is then repressed via chromatin modification. lncRNA may form complex structures that mediate their association with various regulatory proteins. A small molecule that binds to these lncRNA structures could be used to modulate the expression of genes that are normally regulated by an individual lncRNA.

### Targeting Toxic RNA (Repeat RNA)

Simple repeats in mRNA often are associated with human disease. These are often, but not exclusively, repeats of three nucleotides such as CAG (“triplet repeats”) (for reviews, see Gatchel and Zoghbi, Nature Reviews Genetics 6:743-755, 2005; Krzyzosiak et al., Nucleic Acids Res. 40:11-26, 2012; Budworth and McMurray, Methods Mol. Biol. 1010:3-17, 2013, hereby incorporated by reference). Triplet repeats are abundant in the human genome, and they tend to undergo expansion over generations. Approximately 40 human diseases are associated with the expansion of repeat sequences. Diseases caused by triplet expansions are known as Triplet Repeat Expansion Diseases (TRED). Healthy individuals have a variable number of triplet repeats, but there is a threshold beyond which a higher repeat number causes disease. The threshold varies in different disorders. The triplet repeat can be unstable. As the gene is inherited, the number of repeats may increase, and the condition may be more severe or have an earlier onset from generation to generation. When an individual has a number of repeats in the normal range, it is not expected to expand when passed to the next generation. When the repeat number is in the premutation range (a normal, but unstable repeat number), then the repeats may or may not expand upon transmission to the next generation. Normal individuals who carry a premutation do not have the condition but are at risk of having a child who has inherited a triplet repeat in the full mutation range and who will be affected. TREDs can be autosomal dominant, autosomal recessive or X-linked. The more common triplet repeat disorders are autosomal dominant.

The repeats can be in the coding or noncoding portions of the mRNA. In the case of repeats within noncoding regions, the repeats may lie in the 5′ UTR, introns, or 3′ UTR sequences. Some examples of diseases caused by repeat sequences within coding regions are shown in Table

TABLE A
Repeat Expansion Diseases in Which the Repeat
Resides in the Coding Regions of mRNA

| | | | Normal | Disease |
|---|---|---|---|---|
| | | | repeat | repeat |
| Disease | Gene | Repeat | number | number |
| HD | HTT | CAG | 6-35 | 36-250 |
| DRPLA | ATN1 | CAG | 6-35 | 49-88 |
| SBMA | AR | CAG | 9-36 | 38-62 |
| SCA1 | ATXN1 | CAG | 6-35 | 49-88 |
| SCA2 | ATXN2 | CAG | 14-32 | 33-77 |
| SCA3 | ATXN3 | CAG | 12-40 | 55-86 |
| SCA6 | CACNA1A | CAG | 4-18 | 21-30 |
| SCA7 | ATXN7 | CAG | 7-17 | 38-120 |
| SCA17 | TBP | CAG | 25-42 | 47-63 |

In some embodiments, the target RNA is one of those listed in Table A, or a precursor, isoform, fragment, or mutant thereof.

Some examples of diseases caused by repeat sequences within noncoding regions of mRNA are shown in Table B.

TABLE B
Repeat Expansion Diseases in Which the Repeat
Resides in the Noncoding Regions of mRNA

| | | | | Normal | Disease |
|---|---|---|---|---|---|
| | | | Repeat | repeat | repeat |
| Disease | Gene | Repeat | location | number | number |
| Fragile X | FMR1 | CGG | 5′ UTR | 6-53 | ≥230 |
| DM1 | DMPK | CTG | 3′ UTR | 5-37 | ≥50 |
| FRDA | FXN | GAA | Intron | 7-34 | ≥100 |
| SCA8 | ATXN8 | CTG | Noncoding | 16-37 | 110-250 |
| | | | antisense | | |
| SCA10 | ATXN10 | ATTCT | Intron | 9-32 | 800-4500 |
| SCA12 | PPP2R2B | CAG | 5′ UTR | 7-28 | 66-78 |
| C9FTD/ALS | C9orf72 | GGGGCC | Intron | ~30 | 100 s |

In some embodiments, the target RNA is one of those listed in Table B, or a precursor, isoform, fragment, or mutant thereof.

The toxicity that results from the repeat sequence can be direct consequence of the action of the toxic RNA itself, or, in cases in which the repeat expansion is in the coding sequence, due to the toxicity of the RNA and/or the aberrant protein. The repeat expansion RNA can act by sequestering critical RNA-binding proteins (RBP) into foci. One example of a sequestered RBP is the Muscleblind family protein MBNL1. Sequestration of RBPs leads to defects in splicing as well as defects in nuclear-cytoplasmic transport of RNA and proteins. Sequestration of RBPs also can affect miRNA biogenesis. These perturbations in RNA biology can profoundly affect neuronal function and survival, leading to a variety of neurological diseases.

Repeat sequences in RNA form secondary and tertiary structures that bind RBPs and affect normal RNA biology. One specific example disease is myotonic dystrophy (DM1; dystrophia myotonica), a common inherited form of muscle disease characterized by muscle weakness and slow relaxation of the muscles after contraction (Machuca-Tzili et al., Muscle Nerve 32:1-18, 2005, hereby incorporated by reference). It is caused by a CUG expansion in the 3′ UTR of the dystrophia myotonica protein kinase (DMPK) gene. This repeat-containing RNA causes the misregulation of alternative splicing of several developmentally regulated transcripts through effects on the splicing regulators MBNL1 and the CUG repeat binding protein (CELF1) (Wheeler et al., Science 325:336-339, 2009, hereby incorporated by reference). Small molecules that bind the CUG repeat within the DMPK transcript would alter the RNA structure and prevent focus formation and alleviate the effects on these spicing regulators. Fragile X Syndrome (FXS), the most common inherited form of mental retardation, is the consequence of a CGG repeat expansion within the 5′ UTR of the FMR1 gene (Lozano et al., Intractable Rare Dis. Res. 3:134-146, 2014, hereby incorporated by reference). FMRP is critical for the regulation of translation of many mRNAs and for protein trafficking, and it is an essential protein for synaptic development and neural plasticity. Thus, its deficiency leads to neuropathology. A small molecule targeting this CGG repeat RNA may alleviate the suppression of FMR1 mRNA and FMRP protein expression. Another TRED having a very high unmet medical need is Huntington's disease (HD). HD is a progressive neurological disorder with motor, cognitive, and psychiatric changes (Zuccato et al., Physiol Rev. 90:905-981, 2010, hereby incorporated by reference). It is characterized as a poly-glutamine or polyQ disorder since the CAG repeat within the coding sequence of the HTT gene leads to a protein having a poly-glutamine repeat that appears to have detrimental effects on transcription, vesicle trafficking, mitochondrial function, and proteasome activity. However, the HTT CAG repeat RNA itself also demonstrates toxicity, including the sequestration of MBNL1 protein into nuclear inclusions. One other specific example is the GGGGCC repeat expansion in the C9orf72 (chromosome 9 open reading frame 72) gene that is prevalent in both familial frontotemporal dementia (FTD) and amyotrophic lateral sclerosis (ALS) (Ling et al., Neuron 79:416-438, 2013; Haeusler et al., Nature 507:195-200, 2014, hereby incorporated by reference). The repeat RNA structures form nuclear foci that sequester critical RNA binding proteins. The GGGGCC repeat RNA also binds and sequesters RanGAP1 to impair nucleocytoplasmic transport of RNA and proteins (Zhang et al., Nature 525:56-61, 2015, hereby incorporated by reference). Selectively targeting any of these repeat expansion RNAs could add therapeutic benefit in these neurological diseases.

The present invention includes a method of treating a disease or disorder wherein aberrant RNAs themselves cause pathogenic effects, rather than acting through the agency of protein expression or regulation of protein expression. In some embodiments, the target RNA is a repeat RNA, such as those described herein or in Table A or Table B. In some embodiments, the repeat RNA mediates or is implicated in a repeat expansion disease in which the repeat resides in the coding regions of mRNA. In some embodiments, the disease or disorder is a repeat expansion disease in which the repeat resides in the noncoding regions of mRNA. In some embodiments, the disease or disorder is selected from Huntington's disease (HD), dentatorubral-pallidoluysian atrophy (DRPLA), spinal-bulbar muscular atrophy (SBMA), or a spinocerebellar ataxia (SCA) selected from SCA1, SCA2, SCA3, SCA6, SCA7, or SCA17. In some embodiments, the disease or disorder is selected from Fragile X Syndrome, myotonic dystrophy (DM1 or dystrophia myotonica), Friedreich's Ataxia (FRDA), a spinocerebellar ataxia (SCA) selected from SCA8, SCA10, or SCA12, or C9FTD (amyotrophic lateral sclerosis or ALS).

In some embodiments, the disease is amyotrophic lateral sclerosis (ALS), Huntington's disease (HD), frontotemporal dementia (FTD), myotonic dystrophy (DM1 or dystrophia myotonica), or Fragile X Syndrome.

Also provided is a method of producing a small molecule that modulates the activity of a target repeat expansion RNA to treat a disease or disorder, comprising the steps of: screening one or more disclosed compounds for binding to the target repeat expansion RNA; and analyzing the results by an RNA binding assay disclosed herein. In some embodiments, the repeat expansion RNA causes a disease or disorder selected from HD, DRPLA, SBMA, SCA1, SCA2, SCA3, SCA6, SCA7, or SCA17. In some embodiments, the disease or disorder is selected from Fragile X Syndrome, DM1, FRDA, SCA8, SCA10, SCA12, or C9FTD.

### Target RNAs and Diseases/Conditions

An association is known to exist between a large number of RNAs and diseases or conditions, some of which are shown below in Table C or Table D. Accordingly, in some embodiments of the methods described above, the target RNA transcript is selected from one of those in Table C or Table D. In some embodiments, the target RNA mediates or is implicated in a disease or disorder selected from one of those in Table C or Table D. Accordingly, the present invention further provides a method of treating a disease, disorder, or condition selected from one of those in Table C or Table D, comprising the step of administering to a patient in need thereof an effective amount of a disclosed compound. In some embodiments, the method up- or down-regulates the target RNA transcript as shown in the “UP/DOWN REGULATION DESIRABLE?” column in Table C or Table D, below, thus treating the disease, disorder, or condition.

TABLE C
Exemplary Target RNA Transcripts and Associated Diseases

| | | UP/DOWN | | |
|---|---|---|---|---|
| | | REGULATION | Therapeutic | |
| GENE | CLASS | DESIRABLE? | Area | INDICATION(S) |
| MYC | TF | down | Onco | cancer |
| STAT3 | TF | down | Onco | cancer |
| C9orf72 | TRED | down | Neuro | ALS, FTD |
| FOXP3 | TF | down | I&I, I-O | immuno-oncology; I&I |
| MIR155 | miRNA | down | Onco, I&I, Neuro | ALS, fibrosis, cancer |
| APOC3 | apoprotein | down | Cardio | chylomicronemia syndrome |
| JUN | TF | down | I&I | I&I |
| RSV | genomic | down | Viral | RSV |
| KRAS | TF | down | Onco | cancer |
| BCL2L1 | IAP | down | Onco | cancer |
| HIF1A | TF | down | Onco | cancer |
| SMARCA2 | helicase | down | Onco | cancer |
| SNCA | | down | Neuro | PD |
| CCNE1 | cyclin | down | Onco | cancer |
| FOXM1 | TA | down | Onco | cancer |
| MYB | TF | down | Onco | cancer |
| PTPN11 | phosphatase | down | Onco, I&I | cancer, SLE |
| CD40LG | TNF | down | I&I | inflammation |
| NFE2L2 | TF | up | I&I | multiple sclerosis |
| RORC | NHR | down | I&I | |
| ZIKV | genomic | down | Viral | ZIKV |
| DENV | genomic | down | Viral | DENV |
| AR | NHR | down | Onco | prostate cancer |
| ASGR1 | | down | Cardio | CVD (cardiovascular disease) |
| BCL2 | IAP | down | Onco | cancer |
| BDNF | NF | up | Neuro | Huntington's Disease |
| BRD4 | epi | down | Onco | cancer |
| CD40 | TNF | down | | immuno-oncology |
| CD47 | Ig | down | I&I, I-O | immuno-oncology |
| CTLA4 | Ig | down | I&I, I-O | immuno-oncology; I&I |
| CTNNB1 | adhesion | down | Onco | cancer |
| DMPK | TRED | down | Neuro | Myotonic dystrophy type 1 (DM1) |
| EIF4E | IF | down | Onco | cancer |
| FOXA1 | TA | down | Onco | cancer |
| GATA3 | TF | down | Onco | cancer |
| IKZF1 | TF | down | Onco | cancer |
| IKZF3 | TF | down | Onco | cancer |
| IL17A | IL | down | I&I | inflammatory & autoimmune |
| | | | | diseases |
| IL23A | IL | down | I&I | inflammatory & autoimmune |
| | | | | diseases |
| IL6 | IL | down | I&I | rheumatoid arthritis |
| ITGA1 | integrin | down | I&I | RA |
| ITGA5 | integrin | down | Onco | solid tumors |
| ITGAE | integrin | down | I&I | UC, Crohns |
| ITGB2, | integrin | down | I&I | psoriasis |
| ITGAL | | | | |
| ITGB7 | integrin | down | I&I | UC, Crohns |
| MAPT | cytoskeleton | down | Neuro | Alzheimer's disease |
| MAX | TF | down | Onco | cancer |
| MDM2 | E3 | down | Onco | cancer |
| MDM4 | E3 | down | Onco | cancer |
| MIR21 | miRNA | down | Onco | cancer |
| NR4A2 | TF | down | Neuro | PD |
| PTEN | phosphatase | up | Onco | cancer |
| PTPN1 | phosphatase | down | Metab | Type 2 diabetes |
| RUNX1 | TF | down | Onco | cancer |
| SIRPA | glycoprotein | down | I&I, I-O | immuno-oncology |
| SMAD7 | TGF | down | I&I | IBD |
| SOX2 | TF | down | Onco | cancer |
| STAT5A | TF | down | Onco | cancer |
| TERT | telomerase | down | Onco | cancer |
| TGFB1 | TGF | down | Fibrosis | fibrosis |
| TNF | TNF | down | I&I | inflammatory disease |
| TNFRSF11A | TNF | down | | osteoporosis |
| TNFSF11 | TNF | down | | osteoporosis |
| TWIST1 | TF | down | Onco | cancer |
| WNT1 | | | Onco | cancer |
| HepB | | down | Viral | HepB |
| influenza | | down | Viral | influenza |
| DGAT2 | transferase | down | | NASH |
| DNMT3 | DNMT | down | Onco | cancer |
| ERBB3 | pseudokinase | down | Onco | cancer |
| FBXW7 | F-box (E3) | down | Onco | cancer |
| FMR1 | TRED | down | Neuro | Fragile X Syndrome; FTXAS |
| FOS | TF | down | | |
| FXN | TRED | down | Neuro | Friedreich's Ataxia |
| IRAK3 | pseudokinase | down | I&I | I&I |
| MECP2 | TF | up/down | Genetic Disease | Rett Syndrome |
| MIR17HG | miRNA | down | Onco | cancer |
| NF1 | | down | | neurofibromatosis |
| ORAI1 | ion channel | down | I&I | I&I |
| PCSK9 | convertase | down | Cardio | hypercholesterolemia |
| PSMB8 | protease | down | I&I | I&I |
| SKP2 | F-box (E3) | down | Onco | cancer |
| USP1 | protease | down | Onco | cancer |
| USP7 | protease | down | Onco | cancer |
| HIF1A | TF | up | I&I | wound repair & regeneration |
| HOTAIR | IncRNA | down | Onco | cancer |
| IKBKG | | down | I&I | I&I |
| IKK2 | kinase | down | I&I | I&I |
| MALAT1 | IncRNA | down | Onco | cancer |
| PRMT5 | KMT | down | Onco | cancer |
| BCL6 | IAP | down | Onco | cancer |
| GRN | | down | Neuro | neurological diseases |
| ABCA1 | transporter | | Cardio | coronary artery disease |
| ABCB11 | transporter | | | Primary Biliary Sclerosis |
| ABCB4 | transporter | | | Primary Biliary Sclerosis |
| ABCG5 | transporter | | Cardio | coronary artery disease |
| ABCG8 | transporter | | Cardio | coronary artery disease |
| ADIPOQ | hormone | up | Metab | diabetes; obesity; metabolic |
| | | | | syndrome |
| APOA1 | | | Cardio | hypercholesterolemia |
| APOA5 | | | Cardio | hypercholesterolemia |
| ATPA2 | Ca ATPase | up | Genetic Disease | congestive heart failure |
| ATXN1 | TRED | | Neuro | spinocerebellar ataxia 1 |
| ATXN10 | TRED | down | Neuro | spinocerebellar ataxia 10 |
| ATXN2 | TRED | | Neuro | spinocerebellar ataxia 2 |
| ATXN3 | TRED | | Neuro | spinocerebellar ataxia 3 |
| ATXN7 | TRED | | Neuro | spinocerebellar ataxia 7 |
| ATXN8 | TRED | | Neuro | spinocerebellar ataxia 8 |
| BACE1 | protease | down | Neuro | Alzheimer's disease |
| BIRC2 | IAP | down | Onco | cancer |
| BIRC3 | IAP | down | Onco | cancer |
| BIRC5 | IAP | down | Onco | cancer |
| BRCA1 | DNA repair | up | Onco | cancer |
| CACNA1A | ion channel | | Neuro | episodic ataxia type 2 |
| CD247 | TCR | | I&I | I&I |
| CD274 | | down | I-O | immuno-oncology |
| CETP | transfer | down | | cardiovascular |
| CFH | complement | | | macular degeneration |
| CFTR | ion channel | up | Genetic Disease | Cystic Fibrosis |
| CNBP | TRED | down | Neuro | Myotonic dystrophy type 2 (DM2) |
| CNTF | NF | | | macular degeneration |
| DIO2 | deiodinase | | Metab | dyslipidemia |
| DMD | cytoskeleton | | Neuro | Duchenne Muscular Dystrophy; |
| | | | | Becker's MD |
| F7 | protease | up | Hematology | hemophilia |
| F8 | protease | up | Hematology | hemophilia |
| F9 | protease | up | Hematology | hemophilia |
| FGF3 | | down | Genetic Disease | achondroplasia |
| HAMP | | down | Genetic Disease | thalassemia; hereditary |
| | | | | hemochromatosis |
| HAVCR2 | | down | I&I, I-O | inflammatory diseases; immuno- |
| | | | | oncology |
| HBG1, | hemoglobin | up | Hematology | sickle cell anemia; beta-thalassemia |
| HBG2 | | | | |
| HIF1AN | | | Onco | cancer |
| IDH1 | dehydrogenase | down | Onco | cancer |
| IL1 | IL | down | I&I | rheumatoid arthritis |
| IRAK4 | kinase | down | I&I | I&I |
| IRF5 | TF | | I-O | immuno-oncology |
| LAMA1 | ECM | | Genetic Disease | Merosin-deficient congenital |
| | | | | MD (MDCA1) |
| LARGE1 | | | Genetic Disease | Muscular Dystroglycanopathy |
| | | | | Type B,6 |
| LINGO1 | | down | Neuro | neurodegeneration |
| MBNL1 | splice factor | | Neuro | Myotonic Dystrophy |
| MCL1 | IAP | down | Onco | cancer |
| MERTK | kinase | | I&I | Lupus |
| METAP2 | peptidase | down | Onco, I&I | cancer, obesity, autoimmune |
| MTOR | kinase | | Onco | cancer |
| NANOG | TF | | Neuro | neurological diseases |
| NF2 | | | | neurofibromatosis |
| NSD-3 | KMT | down | Onco | cancer |
| PAH | hydroxylase | | Genetic Disease | phenylketonuria |
| PCSK6 | convertase | up | Cardio | hypertension |
| PDCD1 | | | I-O | immuno-oncology |
| PDK1, | kinase | | | polycystic kidney disease |
| PDK2 | | | | |
| PDX1 | TF | | Metab | diabetes |
| PPARGC1A | PPAR | | Neuro | Neurological diseases; obesity |
| PRKAA1 | kinase | | Metab | diabetes |
| PRKAB1 | kinase | | Metab | diabetes |
| PRKAG1 | kinase | | Metab | diabetes |
| RTN4 | | down | Neuro | neurodegeneration |
| RTN4R | | down | Neuro | neurodegeneration |
| SCARB1 | HDL receptor | | Cardio | coronary artery disease |
| SIRT6 | KDAC | down | Onco | cancer |
| SMN2 | | up | Neuro | Spinal Muscular Atrophy |
| SMURF2 | | down | | |
| SORT1 | glycoprotein | | Cardio | coronary artery disease |
| SSPN | cytoskeleton | | Genetic Disease | Duchenne's MD |
| TBX21 | | | I-O | immuno-oncology |
| THRB | NHR | | | dyslipidemia; NASH; NAFLD |
| TNFAIP3 | | | I&I | inflammatory disease; liver failure; |
| | | | | liver transplant |
| TRIB1 | pseudokinase | | Cardio | coronary artery disease |
| TTR | | down | Genetic Disease | amyloidosis |
| UTRN | cytoskeleton | | Genetic Disease | Duchenne Muscular Dystrophy |
| XIAP | IAP | down | Onco | cancer |
| RAGE | | | | |
| ANGPTL3 | | | | |

TABLE D
Additional Target RNA Transcripts

| | | | UP/DOWN | | |
|---|---|---|---|---|---|
| | COMMON | | REGULATION | | |
| GENE | NAME | CLASS | DESIRABLE? | TA | INDICATION(S) |
| CTSL | cathepsin L | protease | up | neuro | PD |
| AR | AR-V7 | NHR | down | cancer | CRPC |
| JMJD6 | JMJD6 | HDM | down | cancer | GBM |
| DNMT1 | DNMT1 | Me-transferase | down | cancer | GBM |
| ASGR1 | ASGR1 | ASG receptor | down | CVD | CVD |
| NAMPT | NAMPT | transferase | down | cancer | various |
| | IRE | | | | |
| ARID1B | ARID1B | | | | |
| SOX10 | SOX10 | | | | |
| HNF1B | TCF2 | | | | |
| PTPN2 | PTPN2 | | | | |
| NLGN3 | NLGN3 | | | | |
| | ETS | | | | |

### 2. Compounds and Related Definitions

It has now been found that the bifunctional compounds of this invention, and pharmaceutically acceptable salts and compositions thereof, are effective as modulators of degradation of a target RNA transcript in a biological sample or patient. Such compounds are also useful in treating an RNA-mediated disease, disorder, or condition, such as those described herein.

As described generally above, the present invention provides a bifunctional compound of Formula A:

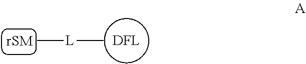

or a pharmaceutically acceptable salt thereof, wherein:
rSM is an RNA-binding small molecule that binds to a target RNA transcript;
DFL is a Decay Factor-recruiting Ligand; and
L is a bivalent linker group that covalently connects the rSM to the DFL;
wherein the DFL binds to or recruits one or more decay factors that degrade the target RNA transcript.
   RNA-Binding Small Molecules (rSMs)

In one aspect, the disclosure provides bifunctional compounds of Formula A wherein the compound includes an rSM. A variety of rSMs known in the art may be used in accordance with the present invention. In some embodiments, the rSM is modified from its known structure in order to covalently attach the rSM to the linker, L, at any available and modifiable C atom or a heteroatom such as an N, O, S, or P atom of the rSM. In the context of a C atom, “modifiable” refers to a C atom having 1) an attached H atom that can be replaced by a bond to L via a chemical reaction such as an oxidation, reduction, nucleophilic substitution, or cross-coupling reaction; or 2) a C atom that can participate in a chemical reaction such as oxidation, reduction, nucleophilic substitution, or cross-couple reaction due to unsaturation or the presence of a leaving group attached to the C atom. For example, a C═O group, C═N group, or C—Br group is “modifiable.” Similarly, a modifiable heteroatom may be attached to an H atom capable of being replaced by a bond to L, or is modifiable due to unsaturation or the presence of a leaving group attached to the heteroatom.

In some embodiments, the rSM is a small molecule or pharmaceutically acceptable salt thereof. In some embodiments, the rSM has a molecular weight (MW) of 1000 or less. In some embodiments, the rSM has a MW of about 750 or less. In some embodiments, the rSM has a MW of about 600 or less. In some embodiments, the rSM has a MW of about 500 or less. In some embodiments, the rSM has a MW of between about 100 and about 1000. In some embodiments, the rSM has a MW of between about 150 and about 800, about 150 and about 600, about 150 and about 400, about 150 and about 350, about 200 and about 350, or between about 200 and about 450.

In some embodiments, the rSM or compound of Formula A binds to the target RNA transcript, or an isoform, fragment, or mutant thereof, with a K_{d} of 1 μM, 500 nM, 100 nM, 50 nM, 10 nM, 1 nM, 500 μM, 10 μM, or 1 μM or lower affinity under biological conditions. In some embodiments, the rSM or compound binds to the target RNA transcript, or an isoform, fragment, or mutant thereof, with a K_{d} of 0.1 nm to 500 nm, 10 nm to 250 nm, 0.001-25 μM, 0.01-25 M, 0.1-25 μM, 0.1-15 μM, 0.01-10 μM, 0.001-1 μM, 0.001-0.1 μM, or 0.001-0.01 μM.

Exemplary rSMs

In some embodiments, the rSM is selected from one of the following:

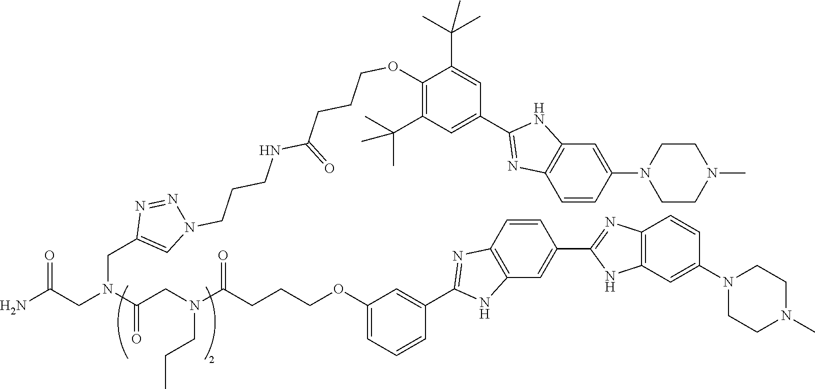

or a pharmaceutically acceptable salt thereof; wherein the rSM is covalently bound to L at any available modifiable C, N, or O atom.

In some embodiments, the rSM is a G-quadruplex binder, such as one of those described in Peng, W. et al., J. Med. Chem. 2018, 61, 6629-6646, which is hereby incorporated by reference.

In some embodiments, the rSM is a compound disclosed in Shi, Y. et al., Cell Chem. Biol. 2019, 26, 319-330, which is hereby incorporated by reference, such as one of the following:

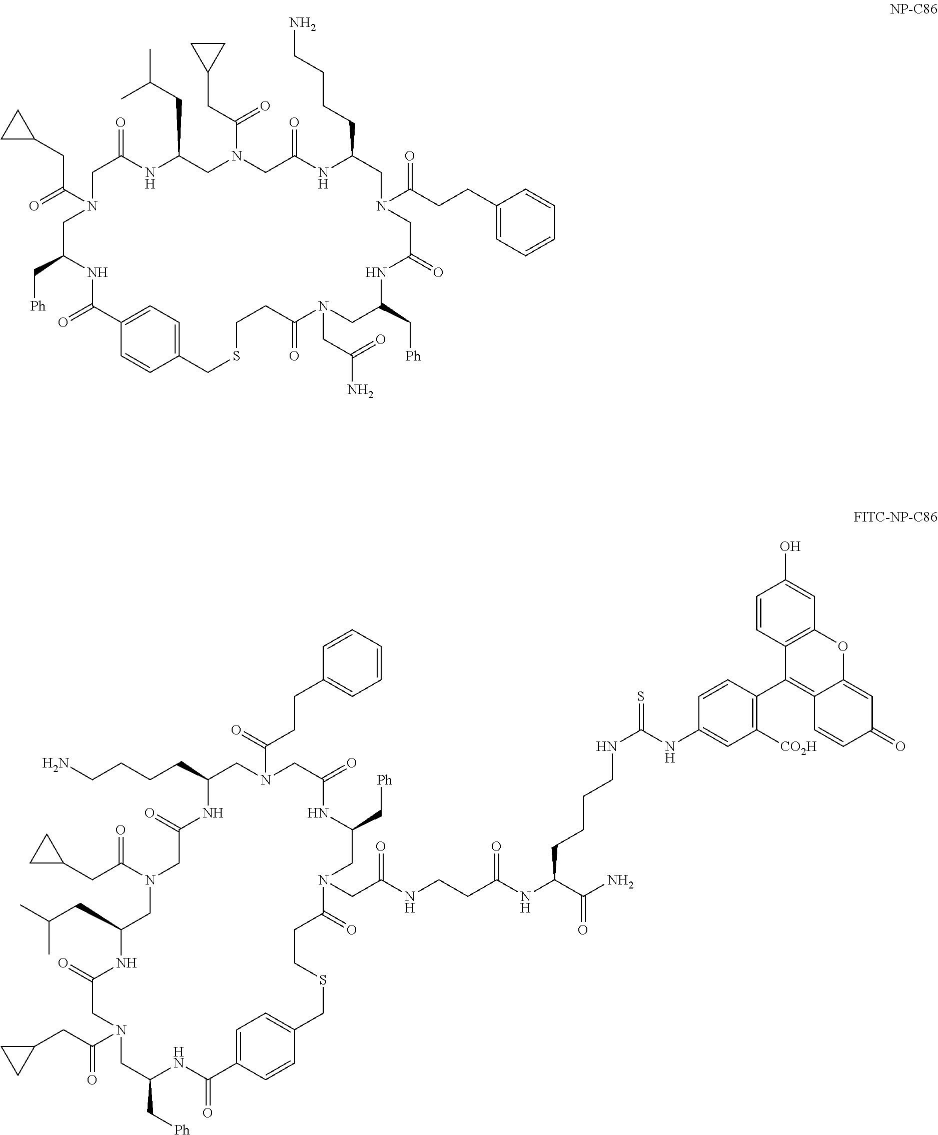

or a pharmaceutically acceptable salt thereof, wherein the rSM is covalently bound to L at any available modifiable C, N, O, S, or P atom.

In some embodiments, the rSM is a compound disclosed in Velagapudi, S. P. et al. (2014), “Sequence-based design of bioactive small molecules that target precursor microRNAs,” Nat. Chem. Biol. 10, 291, hereby incorporated by reference, for example the following:

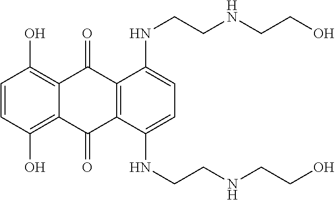 [SMILES: O=C1c2c(O)ccc(O)c2C(=O)c2c(NCCNCCO)ccc(NCCNCCO)c21]

or a pharmaceutically acceptable salt thereof; wherein the rSM is covalently bound to L at any available modifiable C, N, or O atom.

In some embodiments, the rSM is a MALAT-1 binder such as the following:

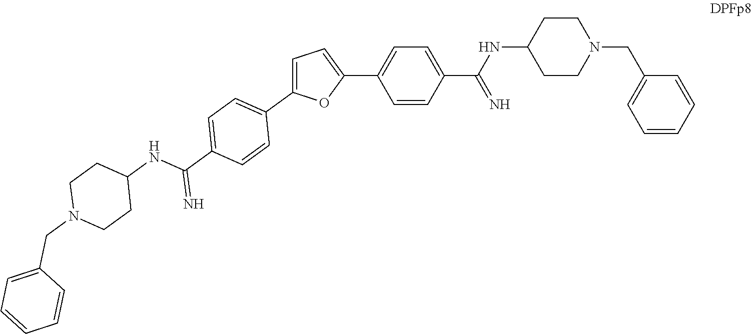 [SMILES: N=C(NC1CCN(Cc2ccccc2)CC1)c1ccc(-c2ccc(-c3ccc(C(=N)NC4CCN(Cc5ccccc5)CC4)cc3)o2)cc1]

or a pharmaceutically acceptable salt thereof; wherein the rSM is covalently bound to L at any available modifiable C, N, or O atom.

In some embodiments, the rSM is a G-quadruplex binder such as the following:

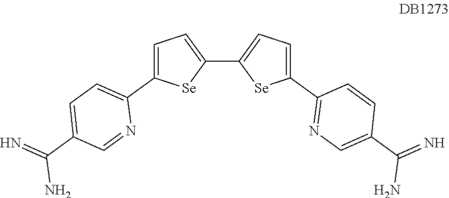 [SMILES: N=C(N)c1ccc(-c2ccc(-c3ccc(-c4ccc(C(=N)N)cn4)[se]3)[se]2)nc1]

or a pharmaceutically acceptable salt thereof; wherein the rSM is covalently bound to L at any available modifiable C or N atom.

In some embodiments, the rSM is one of the following compounds:

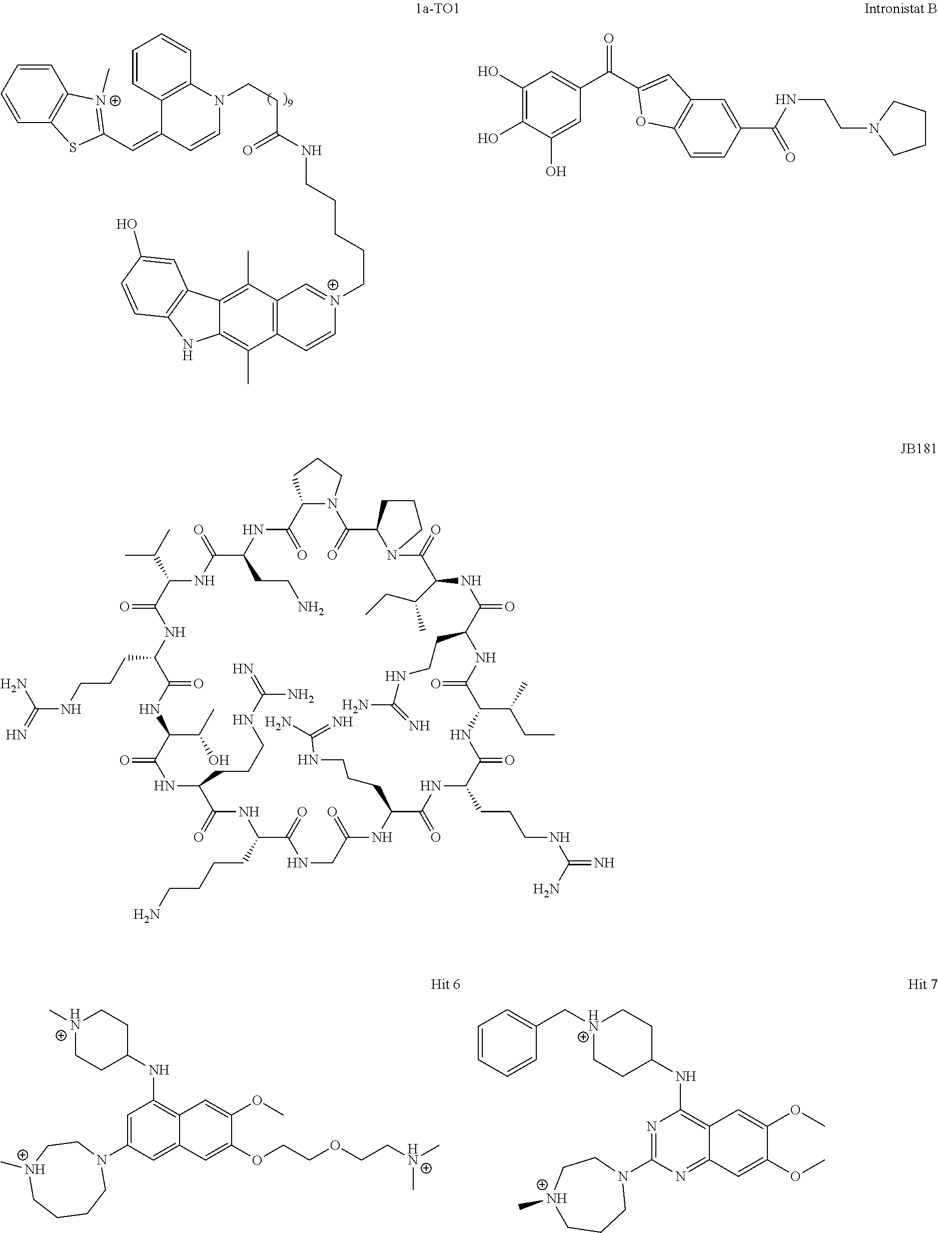

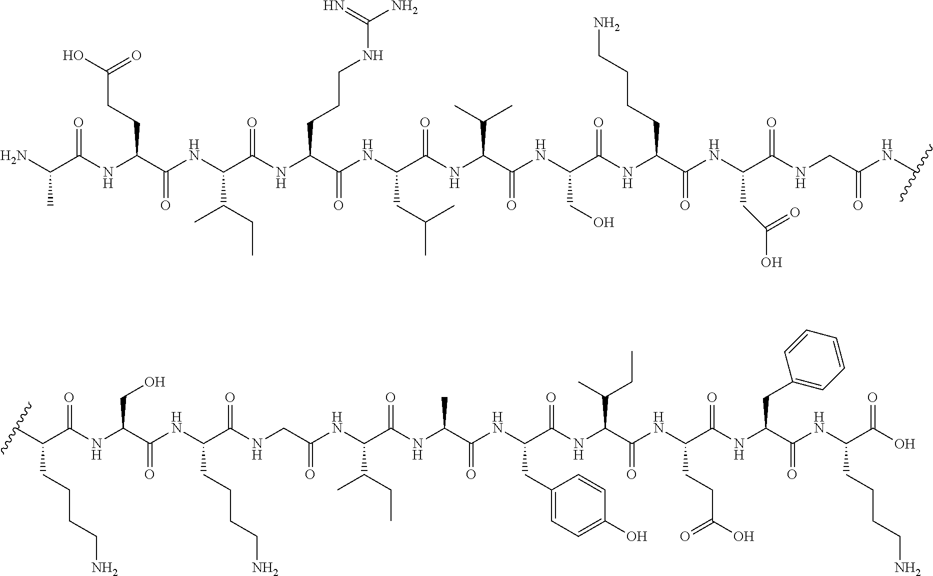 [SMILES: *NC(=O)CNC(=O)[C@H](CC(=O)O)NC(=O)[C@H](CCCCN)NC(=O)[C@H](CO)NC(=O)[C@@H](NC(=O)[C@H](CC(C)C)NC(=O)[C@H](CCCNC(=N)N)NC(=O)[C@@H](NC(=O)[C@H](CCC(=O)O)NC(=O)[C@H](C)N)C(C)CC)C(C)C ; *[C@@H](CCCCN)C(=O)N[C@@H](CO)C(=O)N[C@@H](CCCCN)C(=O)NCC(=O)N[C@H](C(=O)N[C@@H](C)C(=O)N[C@@H](Cc1ccc(O)cc1)C(=O)N[C@H](C(=O)N[C@@H](CCC(=O)O)C(=O)N[C@@H](Cc1ccccc1)C(=O)N[C@@H](CCCCN)C(=O)O)C(C)CC)C(C)CC]

or a pharmaceutically acceptable salt thereof; wherein the rSM is covalently bound to L at any available modifiable C, N, S, or O atom.

In some embodiments, the rSM is selected from one of those described in J. Med. Chem. 2018, 61 (15), 6501-6517, or U.S. Pat. No. 8,729,263, each of which is hereby incorporated by reference. For example, the rSM is selected from a compound according to Formula I from U.S. Pat. No. 8,729,263:

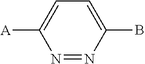

or a pharmaceutically acceptable salt thereof, wherein each variable is as defined therein; and
wherein the rSM is covalently bound to L at any available modifiable C, N, O, S, or P atom.

In some embodiments, the rSM is selected from one of those described in U.S. Pat. No. 9,040,712, which is hereby incorporated by reference. For example, in some embodiments, the rSM is selected from a compound according to Formula X from U.S. Pat. No. 9,040,712:

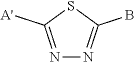

or a pharmaceutically acceptable salt thereof, wherein each variable is as defined therein; and
wherein the rSM is covalently bound to L at any available modifiable C, N, O, S, or P atom.

In some embodiments, the rSM is selected from one of those described in Angelbello, A. J., et al., “Small molecule targeting of RNA structures in neurological disorders,” Annals of the New York Academy of Sciences, 2020 July; 1471 (1): 57-71, hereby incorporated by reference, or a pharmaceutically acceptable salt thereof; wherein the rSM is covalently bound to L at any available modifiable C, N, O, S, or P atom. In some embodiments, the rSM is one of the following:

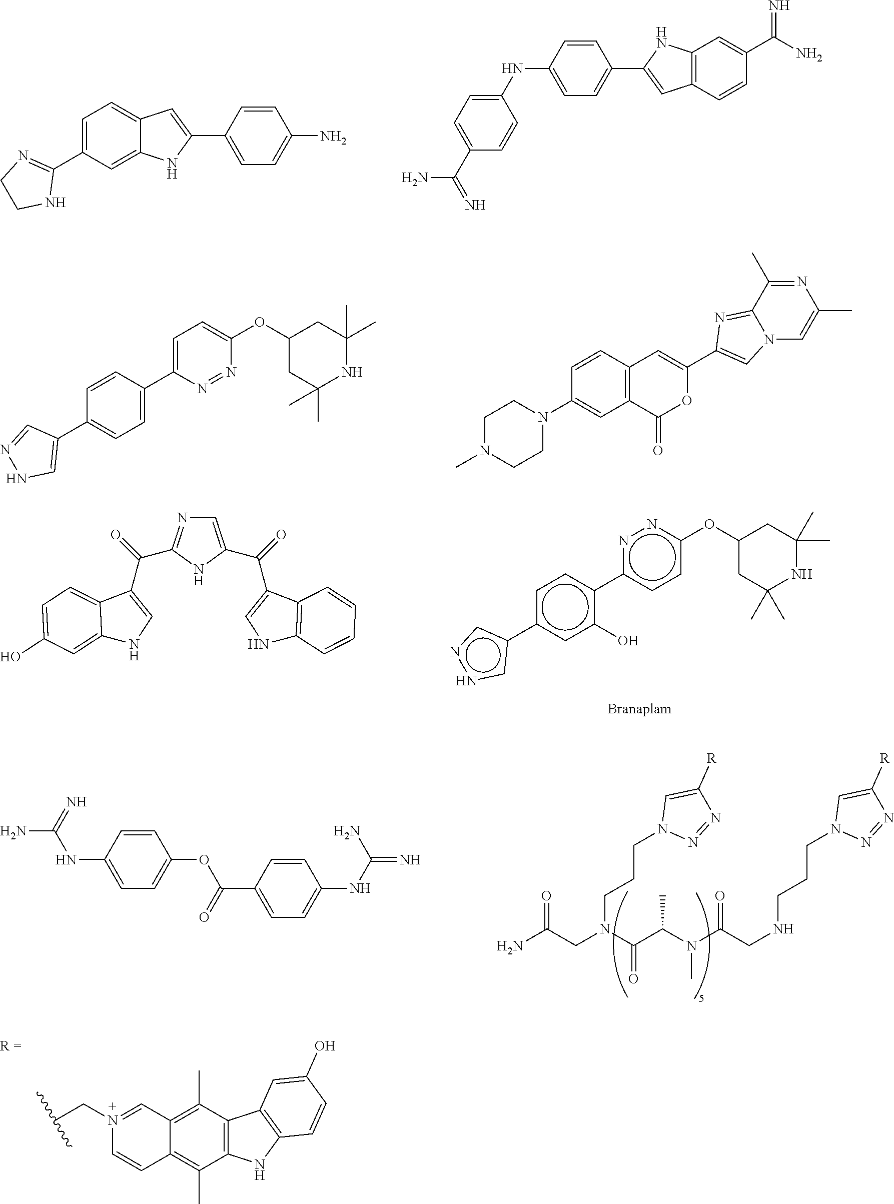

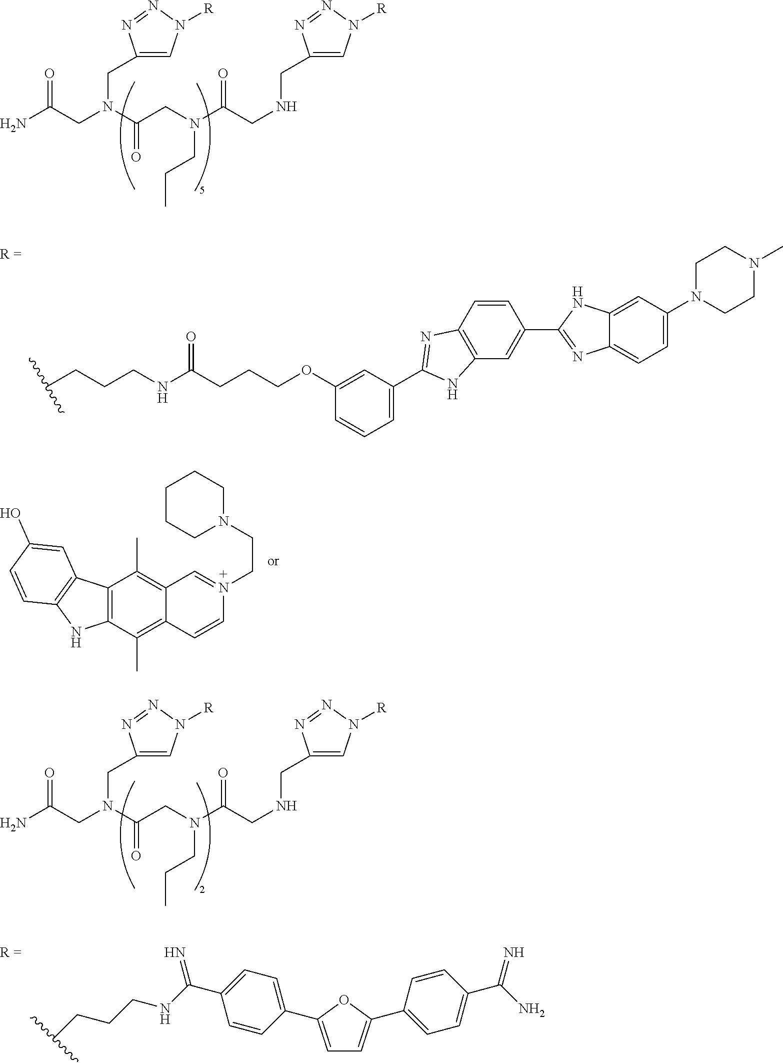

or a pharmaceutically acceptable salt thereof; wherein the rSM is covalently bound to L at any available modifiable C, N, O, S, or P atom.

In some embodiments, the rSM is

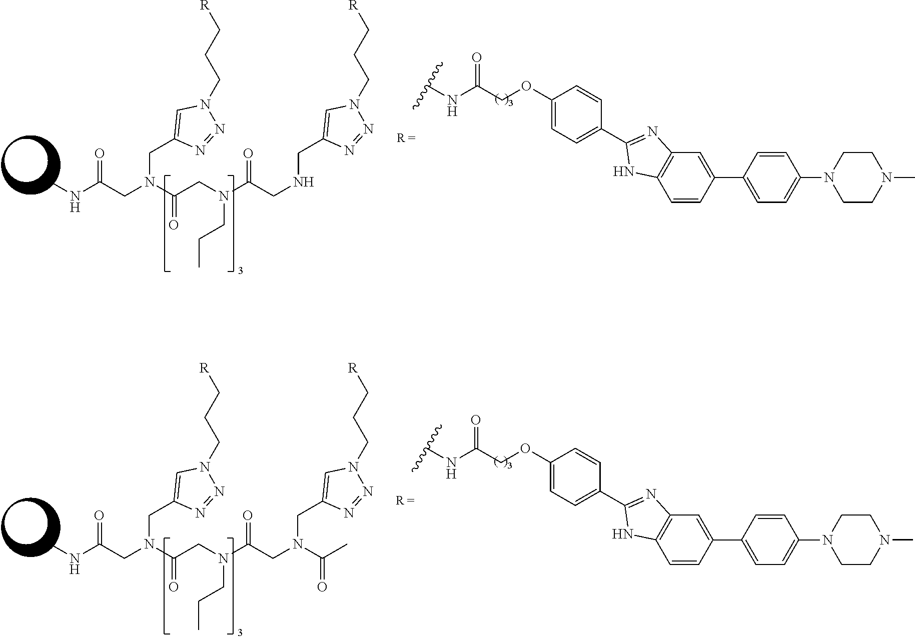

or a pharmaceutically acceptable salt thereof; wherein the rSM is covalently bound to L of Formula A at the shaded ball in the structure above. In some embodiments, the rSM binds to an miRNA such as miR-21.

In some embodiments, the rSM is selected from one of those depicted in Table 1A, below; or a pharmaceutically acceptable salt thereof; wherein the rSM is covalently bound to L at any available modifiable C, N, O, S, or P atom.

In some embodiments, the rSM is a compound according to Formula IX:

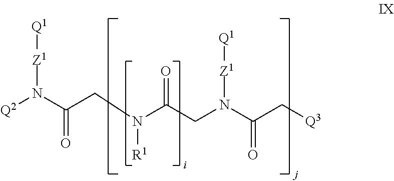

or a pharmaceutically acceptable salt thereof; wherein the rSM is covalently bound to L at any available modifiable C, N, O, S, or P atom; and

wherein each variable is as defined in U.S. Pat. No. 9,150,612, the entirety of which is hereby incorporated by reference.

In some embodiments, the rSM is a compound according to Formula

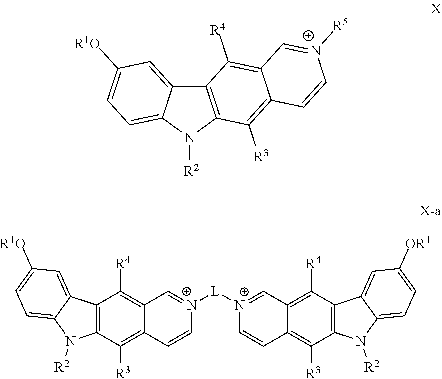

or a pharmaceutically acceptable salt thereof; wherein the rSM is covalently bound to L at any available modifiable C, N, O, S, or P atom; and
wherein each variable is as defined in U.S. Pat. No. 9,550,769, the entirety of which is hereby incorporated by reference. In some embodiments, variable L above is

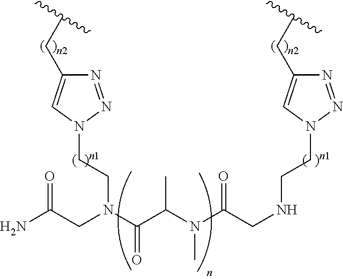

wherein each variable is as defined in U.S. Pat. No. 9,550,769.

In some embodiments, the rSM is selected from one of those disclosed in U.S. Pat. No. 10,157,261, the entirety of which is hereby incorporated by reference; and wherein the rSM is covalently bound to L at any available modifiable C, N, O, S, or P atom.

In some embodiments, the rSM is a compound according to Formula XI:

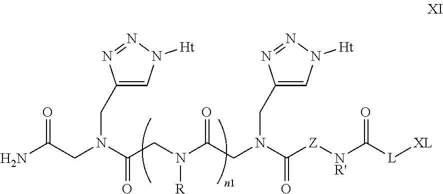

or a pharmaceutically acceptable salt thereof; wherein the rSM is covalently bound to L at any available modifiable C, N, O, S, or P atom; and
wherein each variable is as defined in U.S. Pat. No. 9,586,944, the entirety of which is hereby incorporated by reference.

In some embodiments, the rSM is a compound according to Formula XII:

H—Y—H XII
wherein His a group of the structure

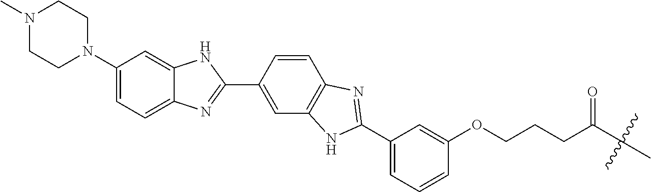 [SMILES: *C(=O)CCCOc1cccc(-c2nc3ccc(-c4nc5ccc(N6CCN(C)CC6)cc5[nH]4)cc3[nH]2)c1]

or a pharmaceutically acceptable salt thereof; wherein the rSM is covalently bound to L at any available modifiable C, N, O, S, or P atom; and
wherein each variable is as defined in U.S. Pat. No. 9,795,687, the entirety of which is hereby incorporated by reference.

In some embodiments, the rSM is a compound selected from one of the following:

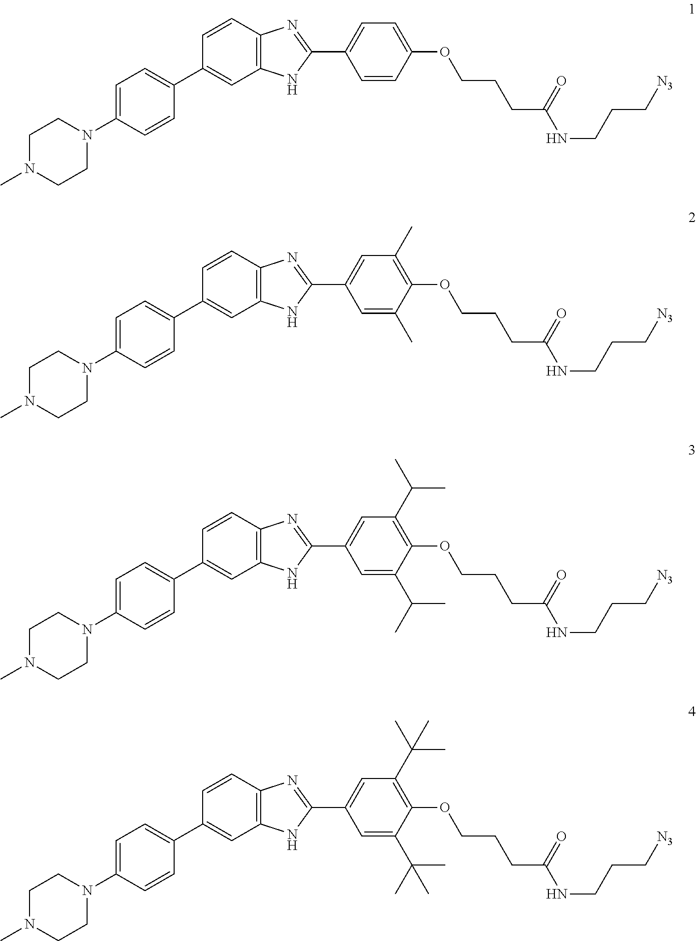 [SMILES: CN1CCN(c2ccc(-c3ccc4nc(-c5ccc(OCCCC(=O)NCCCN=[N+]=[N-])cc5)[nH]c4c3)cc2)CC1 ; Cc1cc(-c2nc3ccc(-c4ccc(N5CCN(C)CC5)cc4)cc3[nH]2)cc(C)c1OCCCC(=O)NCCCN=[N+]=[N-] ; CC(C)c1cc(-c2nc3ccc(-c4ccc(N5CCN(C)CC5)cc4)cc3[nH]2)cc(C(C)C)c1OCCCC(=O)NCCCN=[N+]=[N-] ; CN1CCN(c2ccc(-c3ccc4nc(-c5cc(C(C)(C)C)c(OCCCC(=O)NCCCN=[N+]=[N-])c(C(C)(C)C)c5)[nH]c4c3)cc2)CC1]

or a pharmaceutically acceptable salt thereof; wherein the rSM is covalently bound to L at any available modifiable C, N, O, S, or P atom; or
another compound disclosed in WO 2018/151810, the entirety of which is hereby incorporated by reference.

In some embodiments, the rSM is a compound of the following structure:

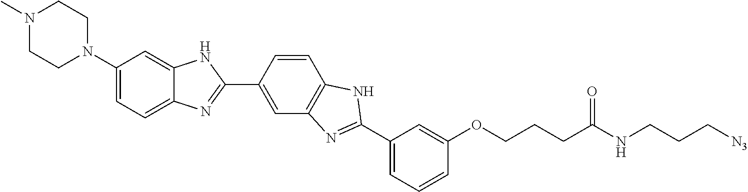 [SMILES: CN1CCN(c2ccc3nc(-c4ccc5[nH]c(-c6cccc(OCCCC(=O)NCCCN=[N+]=[N-])c6)nc5c4)[nH]c3c2)CC1]

or a pharmaceutically acceptable salt thereof; wherein the rSM is covalently bound to L at any available modifiable C, N, O, S, or P atom;
or another compound disclosed in in WO 2018/152414, the entirety of which is hereby incorporated by reference.

In some embodiments, the rSM is a compound of the following structure:

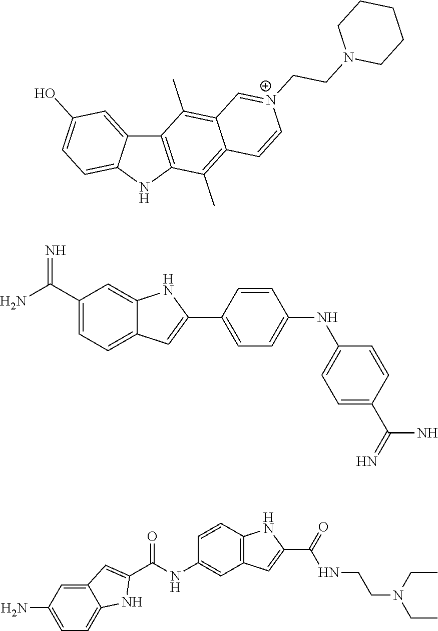 [SMILES: Cc1c2cc[n+](CCN3CCCCC3)cc2c(C)c2c1[nH]c1ccc(O)cc12 ; N=C(N)c1ccc(Nc2ccc(-c3cc4ccc(C(=N)N)cc4[nH]3)cc2)cc1 ; CCN(CC)CCNC(=O)c1cc2cc(NC(=O)c3cc4cc(N)ccc4[nH]3)ccc2[nH]1]

or a pharmaceutically acceptable salt thereof; wherein the rSM is covalently bound to L at any available modifiable C, N, O, S, or P atom; or
another compound disclosed in US 2018/0334678, the entirety of which is hereby incorporated by reference.

In some embodiments, the rSM is a compound according to Formula XIII:

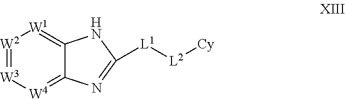

or a pharmaceutically acceptable salt thereof; wherein the rSM is covalently bound to L at any available modifiable C, N, O, S, or P atom; and
wherein each variable is as defined in US 2018/0296532, the entirety of which is hereby incorporated by reference.

In some embodiments, the rSM is a compound according to Formula XIV:

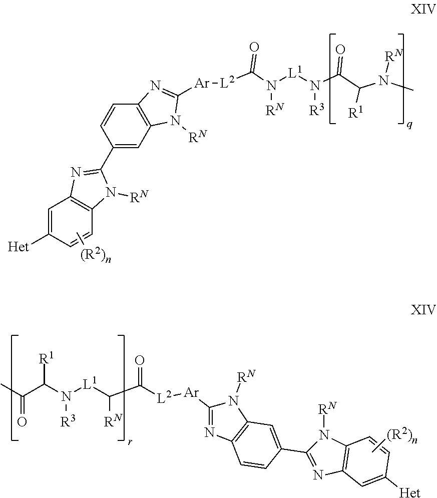

or a pharmaceutically acceptable salt thereof; wherein the rSM is covalently bound to L at any available modifiable C, N, O, S, or P atom; and
wherein each variable is as defined in WO 2018/098297, the entirety of which is hereby incorporated by reference.

In some embodiments, the rSM is a compound according to Formula XV, XVI, or XVII:

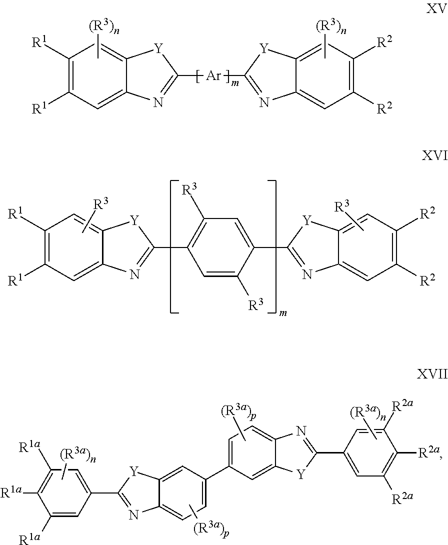

or a pharmaceutically acceptable salt thereof; wherein the rSM is covalently bound to L at any available modifiable C, N, O, S, or P atom; and
wherein each variable is as defined in US 2019/0152924, the entirety of which is hereby incorporated by reference.

In some embodiments, the rSM is a compound according to Formula XVIII:

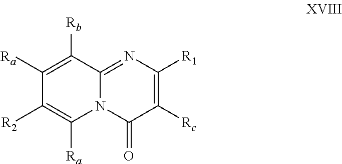

or a pharmaceutically acceptable salt thereof; wherein the rSM is covalently bound to L at any available modifiable C, N, O, S, or P atom; and
wherein each variable is as defined in WO 2019/005993, the entirety of which is hereby incorporated by reference.

In some embodiments, the rSM is a compound according to Formula XIX:

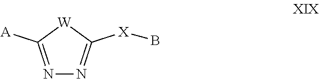

or a pharmaceutically acceptable salt thereof; wherein the rSM is covalently bound to L at any available modifiable C, N, O, S, or P atom; and
wherein each variable is as defined in WO 2018/232039, the entirety of which is hereby incorporated by reference.

In some embodiments, the rSM is a compound according to Formula XX:

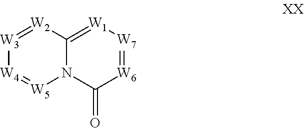

or a pharmaceutically acceptable salt thereof; wherein the rSM is covalently bound to L at any available modifiable C, N, O, S, or P atom; and
wherein each variable is as defined in WO 2019/005980, the entirety of which is hereby incorporated by reference.

In some embodiments, the rSM is a compound according to Formula XXI:

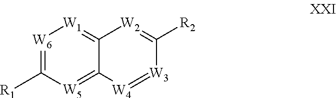

or a pharmaceutically acceptable salt thereof; wherein the rSM is covalently bound to L at any available modifiable C, N, O, S, or P atom; and
wherein each variable is as defined in WO 2018/226622, the entirety of which is hereby incorporated by reference.

In some embodiments, the rSM is a compound according to Formula XXII:

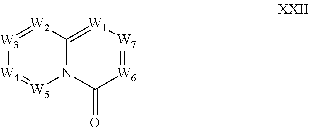

or a pharmaceutically acceptable salt thereof; wherein the rSM is covalently bound to L at any available modifiable C, N, O, S, or P atom; and
wherein each variable is as defined in WO 2018/098446, the entirety of which is hereby incorporated by reference.

In some embodiments, the rSM is a compound according to Formula XXIII:

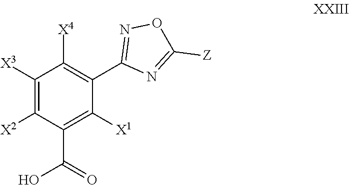

or a pharmaceutically acceptable salt thereof; wherein the rSM is covalently bound to L at any available modifiable C, N, O, S, or P atom; and
wherein each variable is as defined in WO 2017/087364, the entirety of which is hereby incorporated by reference.

In some embodiments, the rSM is ataluren:

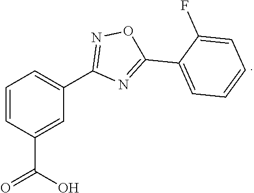 [SMILES: O=C(O)c1cccc(-c2noc(-c3ccccc3F)n2)c1]

or a deuterated analog thereof or pharmaceutically acceptable salt thereof, disclosed in US 2018/0333397 or WO 2017/087364, each of which is hereby incorporated by reference.

In some embodiments, the rSM is a compound of the following structure:

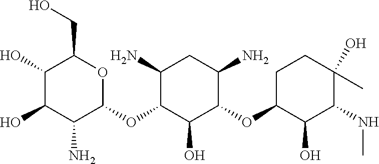 [SMILES: CN[C@@H]1[C@@H](O)[C@@H](O[C@@H]2[C@@H](O)[C@H](O[C@H]3O[C@H](CO)[C@@H](O)[C@H](O)[C@H]3N)[C@@H](N)C[C@H]2N)CC[C@]1(C)O]

or a pharmaceutically acceptable salt thereof, wherein the rSM is covalently bound to L at any available modifiable C, N, O, S, or P atom; as described in US 2018/147228, the entirety of which is hereby incorporated by reference.

In some embodiments, the rSM is a compound according to Formula XXIV:

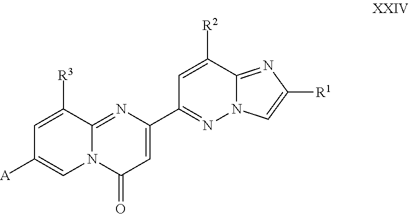

or a pharmaceutically acceptable salt thereof; wherein the rSM is covalently bound to L at any available modifiable C, N, O, S, or P atom; and
wherein each variable is as defined in U.S. Pat. No. 9,969,754, the entirety of which is hereby incorporated by reference.

In some embodiments, the rSM is a compound according to Formula XXV-i:

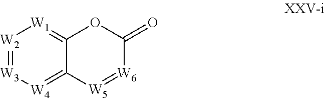

or a pharmaceutically acceptable salt thereof; wherein the rSM is covalently bound to L at any available modifiable C, N, O, S, or P atom; and
wherein each variable is as defined in U.S. Pat. No. 9,371,336, the entirety of which is hereby incorporated by reference. In some embodiments, the rSM is a compound disclosed in U.S. Pat. No. 9,371,336, or a pharmaceutically acceptable salt thereof.

In some embodiments, the rSM is a compound according to Formula XXV-ii:

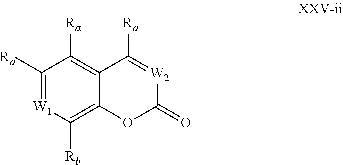

or a pharmaceutically acceptable salt thereof; wherein the rSM is covalently bound to L at any available modifiable C, N, O, S, or P atom; and
wherein each variable is as defined in U.S. Pat. No. 9,617,268, the entirety of which is hereby incorporated by reference. In some embodiments, the rSM is a compound disclosed in U.S. Pat. No. 9,617,268, or a pharmaceutically acceptable salt thereof.

In some embodiments, the rSM is a compound according to Formula XXVI:

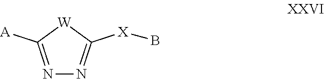

or a pharmaceutically acceptable salt thereof; wherein the rSM is covalently bound to L at any available modifiable C, N, O, S, or P atom; and
wherein each variable is as defined in US 2019/0000844, the entirety of which is hereby incorporated by reference. In some embodiments, the rSM is a compound disclosed in US 2019/0000844, or a pharmaceutically acceptable salt thereof.

In some embodiments, the rSM is a compound according to Formula XXVII:

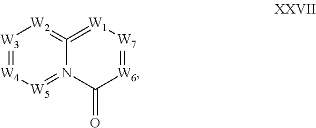

or a pharmaceutically acceptable salt thereof; wherein the rSM is covalently bound to L at any available modifiable C, N, O, S, or P atom; and
wherein each variable is as defined in US 2018/0161456, the entirety of which is hereby incorporated by reference. In some embodiments, the rSM is a compound disclosed in US 2018/0161456, or a pharmaceutically acceptable salt thereof.

In some embodiments, the rSM is a compound according to Formula XXVIII:

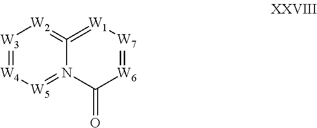

or a pharmaceutically acceptable salt thereof; wherein the rSM is covalently bound to L at any available modifiable C, N, O, S, or P atom; and
wherein each variable is as defined in U.S. Pat. No. 10,195,202, the entirety of which is hereby incorporated by reference. In some embodiments, the rSM is a compound disclosed in U.S. Pat. No. 10,195,202, or a pharmaceutically acceptable salt thereof.

In some embodiments, the rSM is a compound according to one of Formulae XXIX-XXXIII:

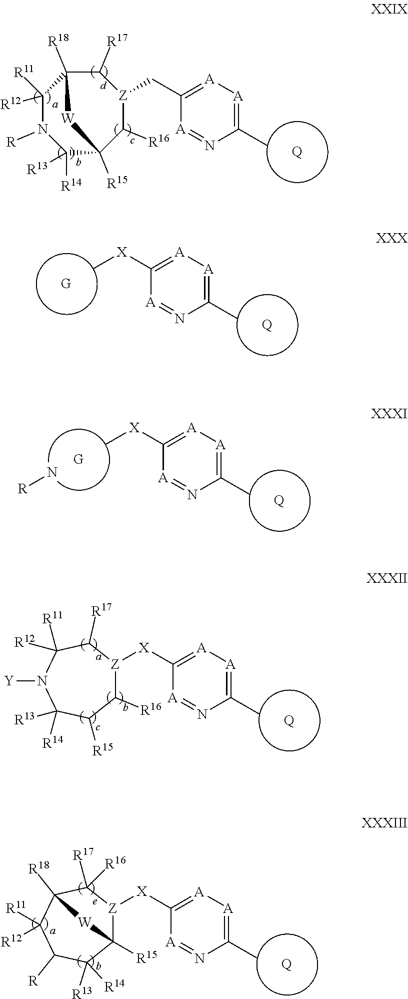

or a pharmaceutically acceptable salt thereof; wherein the rSM is covalently bound to L at any available modifiable C, N, O, S, or P atom; and
wherein each variable is as defined in WO 2019/028440, the entirety of which is hereby incorporated by reference. In some embodiments, the rSM is a compound disclosed in WO 2019/028440, or a pharmaceutically acceptable salt thereof.

In some embodiments, the rSM is a compound according to one of Formulae XXXIV-XLXI:

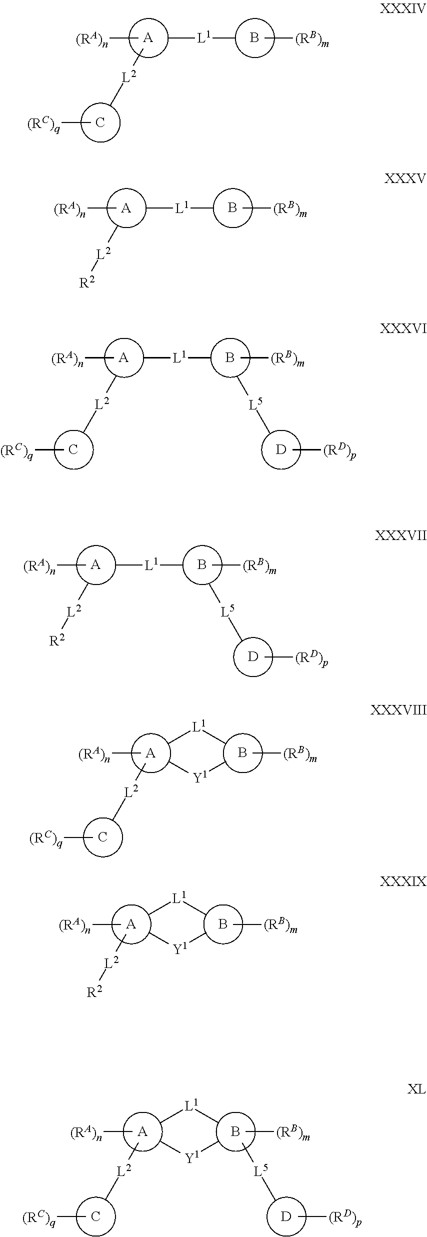

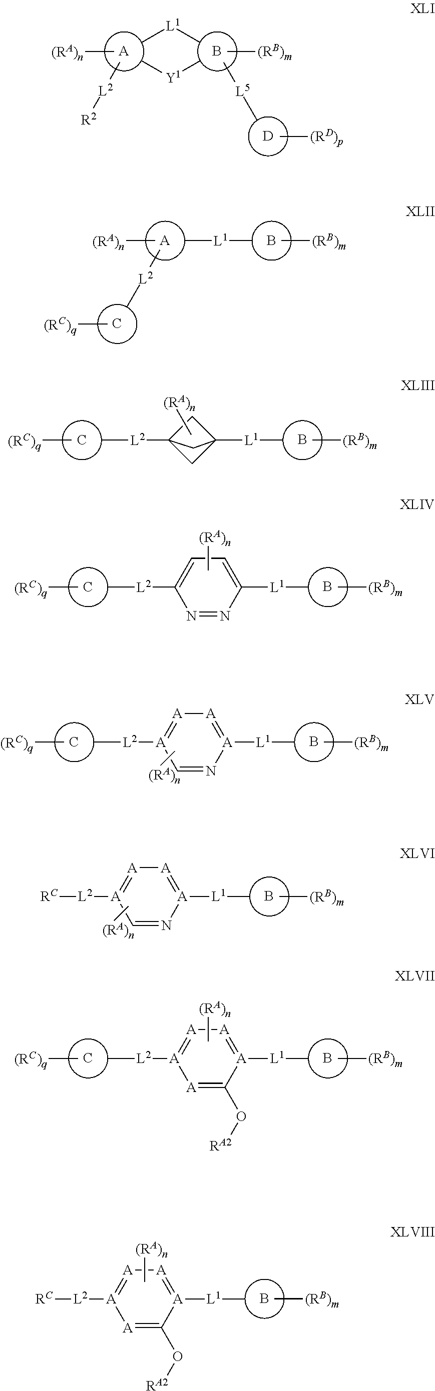

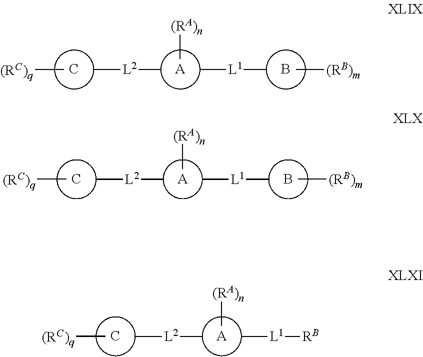

or a pharmaceutically acceptable salt thereof; wherein the rSM is covalently bound to L at any available modifiable C, N, O, S, or P atom; and
wherein each variable is as defined in WO 2019/060917, the entirety of which is hereby incorporated by reference. In some embodiments, the rSM is a compound disclosed in WO 2019/060917, or a pharmaceutically acceptable salt thereof.

In some embodiments, the rSM is a compound according to Formula XLXII or XLXIII:

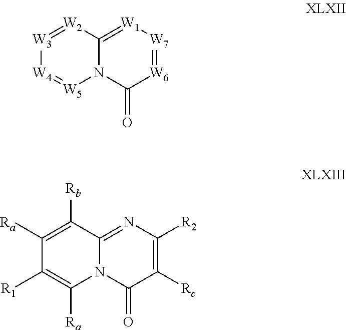

or a pharmaceutically acceptable salt thereof; wherein the rSM is covalently bound to L at any available modifiable C, N, O, S, or P atom; and
wherein each variable is as defined in U.S. Pat. No. 9,879,007, the entirety of which is hereby incorporated by reference.

In some embodiments, the rSM is a compound according to Formula XLXIV or XLXV:

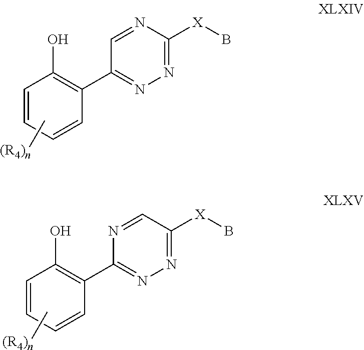

or a pharmaceutically acceptable salt thereof; wherein the rSM is covalently bound to L at any available modifiable C, N, O, S, or P atom; and
wherein each variable is as defined in WO 2019/191229, the entirety of which is hereby incorporated by reference.

In some embodiments, the rSM is a compound according to Formula XLXVI:

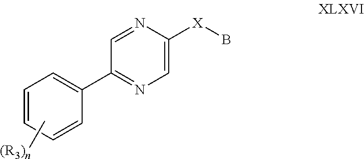

or a pharmaceutically acceptable salt thereof; wherein the rSM is covalently bound to L at any available modifiable C, N, O, S, or P atom; and
wherein each variable is as defined in WO 2019/191092, the entirety of which is hereby incorporated by reference.

In some embodiments, the rSM is a compound according to Formula XLXVII:

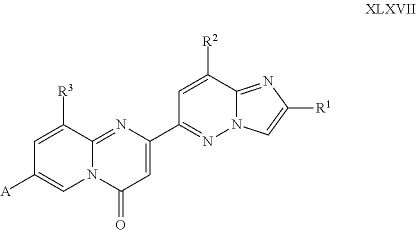

or a pharmaceutically acceptable salt thereof; wherein the rSM is covalently bound to L at any available modifiable C, N, O, S, or P atom; and
wherein each variable is as defined in US 2019/315773, the entirety of which is hereby incorporated by reference.

In some embodiments, the rSM is a compound according to Formula LVIII, LIX, or LX:

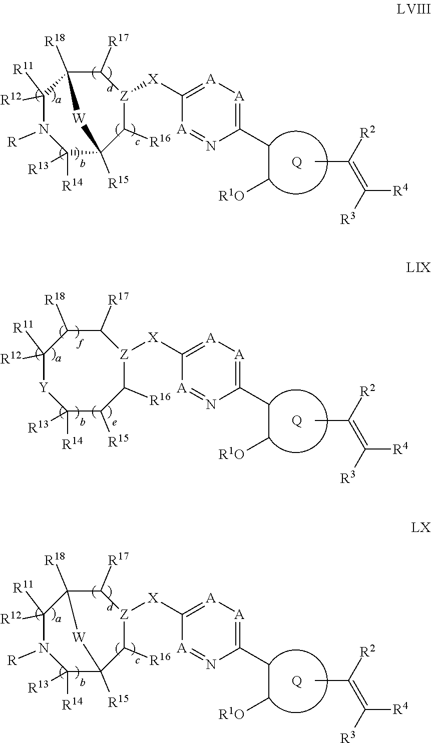

or a pharmaceutically acceptable salt thereof; wherein the rSM is covalently bound to L at any available modifiable C, N, O, S, or P atom; and
wherein each variable is as defined in WO 2019/199972, the entirety of which is hereby incorporated by reference. Such compounds are useful, for example, in modulating splicing of the FOXM1 gene for use in the treatment of cancer.

In some embodiments, the rSM is a compound according to Formula LXI:

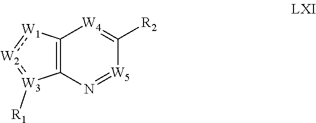

or a pharmaceutically acceptable salt thereof; wherein the rSM is covalently bound to L at any available modifiable C, N, O, S, or P atom; and
wherein each variable is as defined for Formula (I) in WO 2020/005873, the entirety of which is hereby incorporated by reference. Such compounds are useful, for example, in modulating RNA targets that mediate Huntington's disease. In some embodiments, the compound is of formula (Ibb1) described therein:

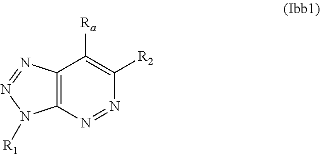

wherein or a pharmaceutically acceptable salt thereof; wherein the rSM is covalently bound to L at any available modifiable C, N, O, S, or P atom; and wherein each variable is as defined therein.

In some embodiments, the rSM is a compound according to Formula LXII or LXIII:

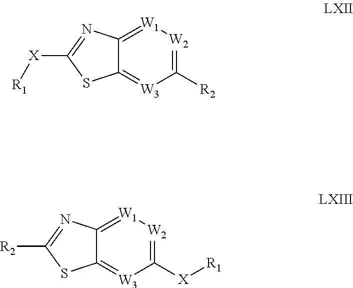

or a pharmaceutically acceptable salt thereof; wherein the rSM is covalently bound to L at any available modifiable C, N, O, S, or P atom; and
wherein each variable is as defined in WO 2020/005877, the entirety of which is hereby incorporated by reference. Such compounds are useful, for example, in binding to HTT RNA transcripts for use in the treatment of diseases such as Huntington's.

In some embodiments, the rSM is a compound according to Formula LXIV, LXV, LXVI, or LXVII:

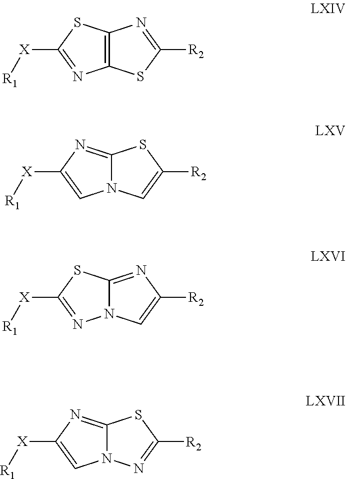

or a pharmaceutically acceptable salt thereof; wherein the rSM is covalently bound to L at any available modifiable C, N, O, S, or P atom; and wherein each variable is as defined in WO 2020/005882, the entirety of which is hereby incorporated by reference. Such compounds are useful, for example, in binding to HTT RNA transcripts for use in the treatment of diseases such as Huntington's.

In some embodiments, the rSM is selected from one of those depicted in U.S. Pat. Nos. 8,729,263, 9,545,404, 9,856,474, or 7,838,657, each of which is hereby incorporated by reference.

In some embodiments, the rSM is selected from one of those depicted in Table 1A, below; or a pharmaceutically acceptable salt thereof; wherein the rSM is covalently bound to L at any available modifiable C, N, O, S, or P atom.

TABLE 1A
Additional rSMs

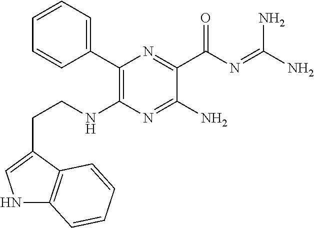 [SMILES: NC(N)=NC(=O)c1nc(-c2ccccc2)c(NCCc2c[nH]c3ccccc23)nc1N]

Patwardhan et al., Med. Chem. Comm. 2017,
8, 1022-1036; hereby incorporated by
reference.

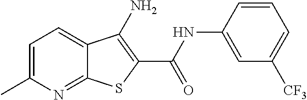 [SMILES: Cc1ccc2c(N)c(C(=O)Nc3cccc(C(F)(F)F)c3)sc2n1]

Sztuba-Solinska et al., J. Am. Chem. Soc.
2014, 136, 8402-8410; hereby incorporated
by reference.

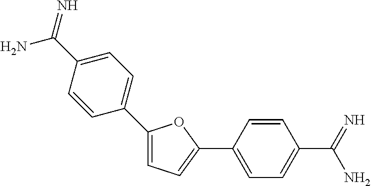 [SMILES: N=C(N)c1ccc(-c2ccc(-c3ccc(C(=N)N)cc3)o2)cc1]

Donlic et al. Angew. Chem. int. Ed. 2018, 40
13242-13247; hereby incorporated by
reference

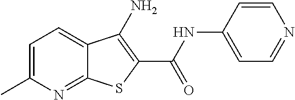 [SMILES: Cc1ccc2c(N)c(C(=O)Nc3ccncc3)sc2n1]

Sztuba-Solinska et al., J. Am. Chem. Soc.
2014, 136, 8402-8410.

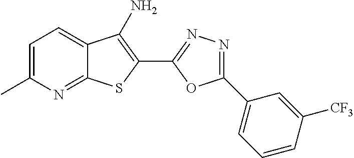 [SMILES: Cc1ccc2c(N)c(-c3nnc(-c4cccc(C(F)(F)F)c4)o3)sc2n1]

Sztuba-Solinska et al., J. Am. Chem. Soc.
2014, 136, 8402-8410.

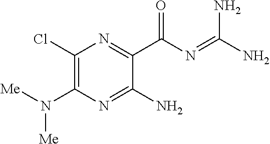 [SMILES: CN(C)c1nc(N)c(C(=O)N=C(N)N)nc1Cl]

Patwardhan et al. 2017

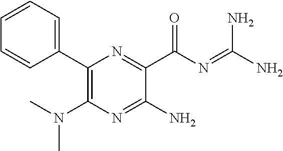 [SMILES: CN(C)c1nc(N)c(C(=O)N=C(N)N)nc1-c1ccccc1]

Patwardhan et al. 2017

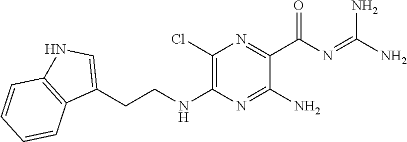 [SMILES: NC(N)=NC(=O)c1nc(Cl)c(NCCc2c[nH]c3ccccc23)nc1N]

Patwardhan et al. 2017

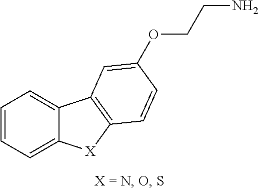

Connelly, et al. Nature communications,
2019; 10(1):1501; hereby incorporated by reference

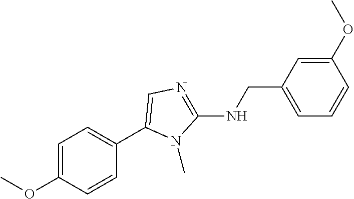 [SMILES: COc1ccc(-c2cnc(NCc3cccc(OC)c3)n2C)cc1]

Abulwerdi; et al., ACS Chem. Biol. 2019, 14, 223-235
hereby incorpoartaed by reference

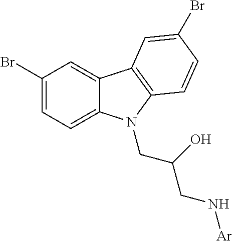

Ar = carbocyclic, heterocyclic
Connelly et al., ACS Chem Biol. 2017, 12(2):
435-443; hereby incorporated by reference.

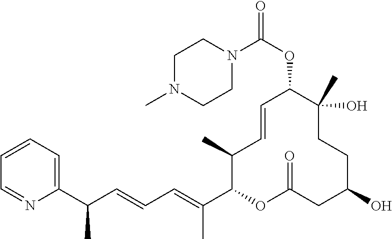 [SMILES: C/C(=C\C=C\[C@@H](C)c1ccccn1)[C@H]1OC(=O)C[C@H](O)CC[C@@](C)(O)[C@@H](OC(=O)N2CCN(C)CC2)/C=C/[C@@H]1C]

Seiler et al., Nature Medicine 2018, 24, 497-504;
hereby incorporated by reference.

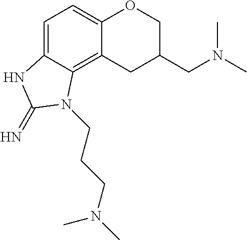 [SMILES: CN(C)CCCn1c(=N)[nH]c2ccc3c(c21)CC(CN(C)C)CO3]

Charrette et al., ACS Chem Biol. 2016,
11(12), 3263-3267; hereby incorporated by reference.

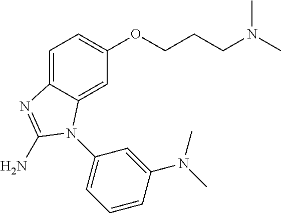 [SMILES: CN(C)CCCOc1ccc2nc(N)n(-c3cccc(N(C)C)c3)c2c1]

Charette et al. 2016

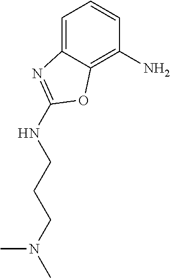 [SMILES: CN(C)CCCNc1nc2cccc(N)c2o1]

Charette et al. 2016

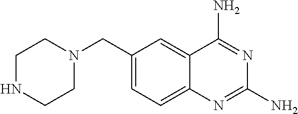 [SMILES: Nc1nc(N)c2cc(CN3CCNCC3)ccc2n1]

Charette et al. 2016

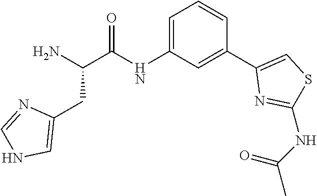 [SMILES: CC(=O)Nc1nc(-c2cccc(NC(=O)[C@@H](N)Cc3c[nH]cn3)c2)cs1]

Joly, J..P. et al., Chemistry 2014, 20, 2071-2079;
hereby incorporated by reference.

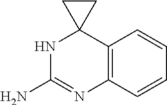 [SMILES: NC1=Nc2ccccc2C2(CC2)N1]

Charette et al. 2016

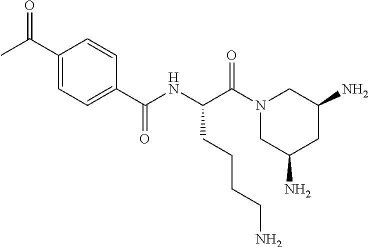 [SMILES: CC(=O)c1ccc(C(=O)N[C@@H](CCCCN)C(=O)N2C[C@H](N)C[C@H](N)C2)cc1]

Charette et al. 2016

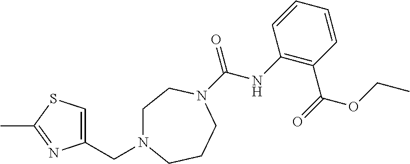 [SMILES: CCOC(=O)c1ccccc1NC(=O)N1CCCN(Cc2csc(C)n2)CC1]

Park et al., J. Am. Chem. Soc. 2011, 133
10094-10100: hereby incorporated by reference

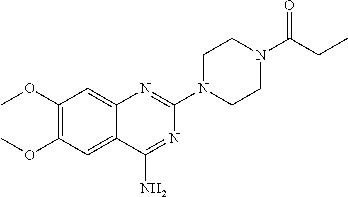 [SMILES: CCC(=O)N1CCN(c2nc(N)c3cc(OC)c(OC)cc3n2)CC1]

Bottini, A. et al., Chem. Biol. Drug Des.
2015, 86, 663-673; hereby incorporated by
reference.

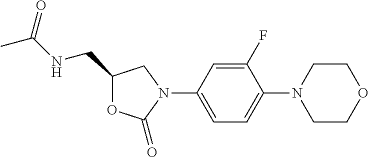 [SMILES: CC(=O)NC[C@H]1CN(c2ccc(N3CCOCC3)c(F)c2)C(=O)O1]

Lin et al., Antimicrob. Agents
Chemother. 1997, 41, 2127-2131; hereby
incorporated by reference.

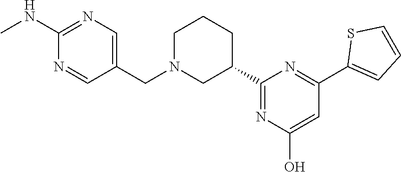 [SMILES: CNc1ncc(CN2CCC[C@H](c3nc(O)cc(-c4cccs4)n3)C2)cn1]

Howe, J. A. et al., Nature 2015, 526, 672-
677; hereby incorporated by reference.

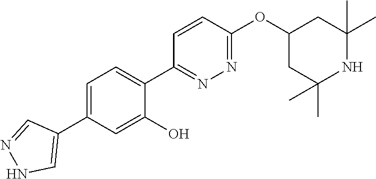 [SMILES: CC1(C)CC(Oc2ccc(-c3ccc(-c4cn[nH]c4)cc3O)nn2)CC(C)(C)N1]

Palacino, J. et al., Nat. Chem. Biol. 2015, 11,
511-517; hereby incorporated by reference.

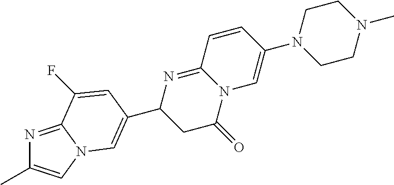

Sivaramakrishnan, M. et al., Nat. Commun.
2017, 8, 1476; hereby incorporated by
reference.

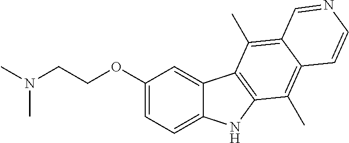 [SMILES: Cc1c2ccncc2c(C)c2c1[nH]c1ccc(OCCN(C)C)cc12]

R. V. Brown, et al., J. Am. Chem. Soc. 2017,
139, 7456-7475; hereby incorporated by
reference.

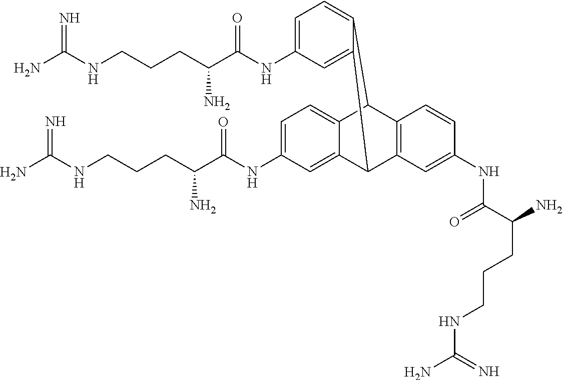 [SMILES: N=C(N)NCCC[C@@H](N)C(=O)Nc1ccc2c(c1)C1c3cc(NC(=O)[C@H](N)CCCNC(=N)N)ccc3C2c2ccc(NC(=O)[C@@H](N)CCCNC(=N)N)cc21]

Barros, S. A., et al., Angew. Chem. Int. Ed.
Engl. 2016, 55, 8258-8261; hereby
incorporated by reference.

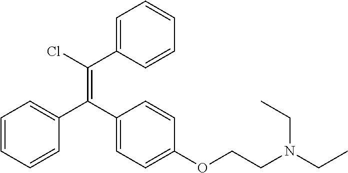 [SMILES: CCN(CC)CCOc1ccc(/C(=C(/Cl)c2ccccc2)c2ccccc2)cc1]

Prado, S. et al., Biochem. Pharmacol. 2016,
107, 14-28; hereby incorporated by reference.

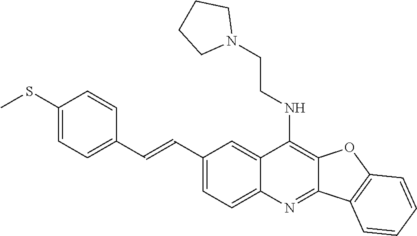 [SMILES: CSc1ccc(/C=C/c2ccc3nc4c(oc5ccccc54)c(NCCN4CCCC4)c3c2)cc1]

Peng et al., J. Med. Chem. 2018, 61, 6629-
6646; hereby incorporated by reference.

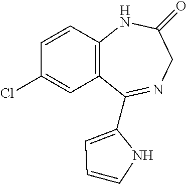 [SMILES: O=C1CN=C(c2ccc[nH]2)c2cc(Cl)ccc2N1]

Mei, H. Y., et al., Bioorg Med Chem. 1997,
5:1173-1184; hereby incorporated by
reference.

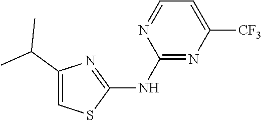 [SMILES: CC(C)c1csc(Nc2nccc(C(F)(F)F)n2)n1]

Garavis, M., et al., ACS Chem. Biol. 2014, 9,
1559-1566; hereby incorporated by
reference.

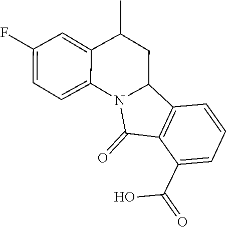 [SMILES: CC1CC2c3cccc(C(=O)O)c3C(=O)N2c2ccc(F)cc21]

Garavis, M., et al., 2014

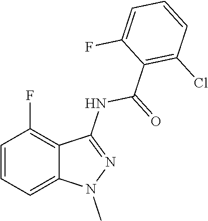 [SMILES: Cn1nc(NC(=O)c2c(F)cccc2Cl)c2c(F)cccc21]

Garavis, M., et al., 2014

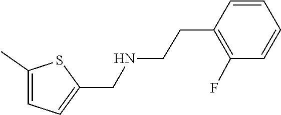 [SMILES: Cc1ccc(CNCCc2ccccc2F)s1]

Garavis, M., et al., 2014

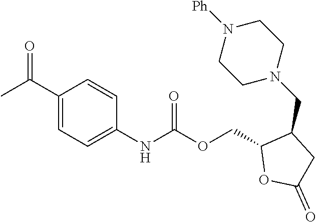 [SMILES: CC(=O)c1ccc(NC(=O)OC[C@H]2OC(=O)C[C@@H]2CN2CCN(c3ccccc3)CC2)cc1]

Orac, C. M., et al., J. Med. Chem. 2011, 54,
6786-6795; hereby incorporated by
reference.

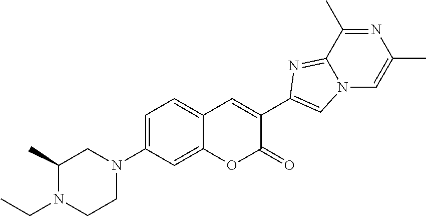 [SMILES: CCN1CCN(c2ccc3cc(-c4cn5cc(C)nc(C)c5n4)c(=O)oc3c2)C[C@@H]1C]

Wang et al., PNAS 2018, 115 (20), E4604-
E4612; hereby incorporated by reference.

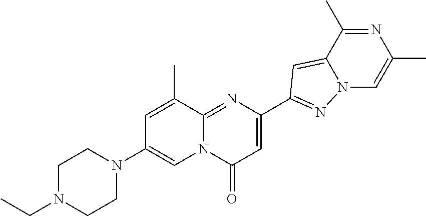 [SMILES: CCN1CCN(c2cc(C)c3nc(-c4cc5c(C)nc(C)cn5n4)cc(=O)n3c2)CC1]

Wang et al., 2018

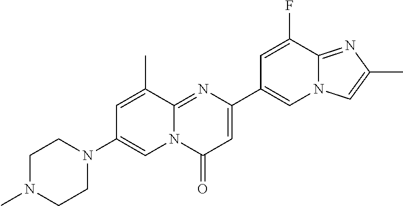

Wang et al., 2018

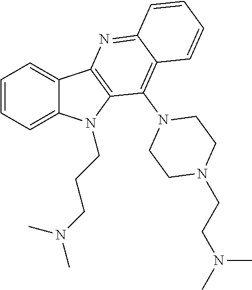 [SMILES: CN(C)CCCn1c2ccccc2c2nc3ccccc3c(N3CCN(CCN(C)C)CC3)c21]

L.D. Smith et al., Cell Rep. 2014, 9, 193-205;
hereby incorporated by reference

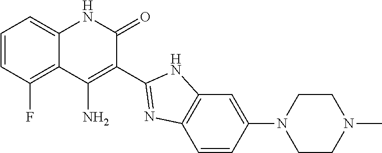 [SMILES: CN1CCN(c2ccc3nc(-c4c(N)c5c(F)cccc5[nH]c4=O)[nH]c3c2)CC1]

Velagapudi, et al., Cell Chem. Bio. 2018, 25,
1086-1094; hereby incorporated by reference.

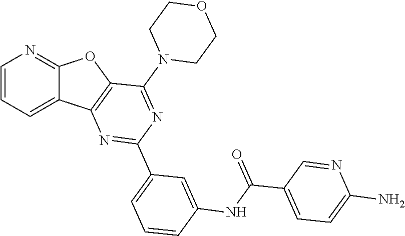 [SMILES: Nc1ccc(C(=O)Nc2cccc(-c3nc(N4CCOCC4)c4oc5ncccc5c4n3)c2)cn1]

Velagapudi, et al., 2018

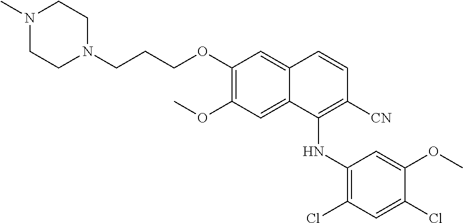

Velagapudi, et al., 2018

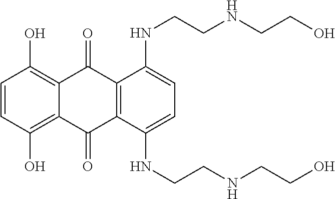 [SMILES: O=C1c2c(O)ccc(O)c2C(=O)c2c(NCCNCCO)ccc(NCCNCCO)c21]

Velagapudi, et al., 2018

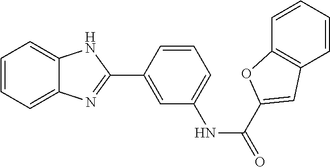 [SMILES: O=C(Nc1cccc(-c2nc3ccccc3[nH]2)c1)c1cc2ccccc2o1]

Velagapudi, et al., 2018

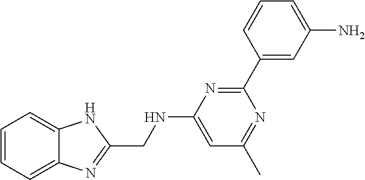 [SMILES: Cc1cc(NCc2nc3ccccc3[nH]2)nc(-c2cccc(N)c2)n1]

Velagapudi, et al., 2018

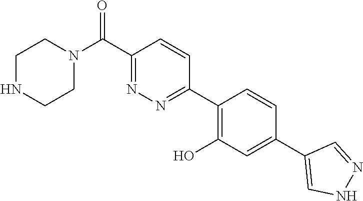 [SMILES: O=C(c1ccc(-c2ccc(-c3cn[nH]c3)cc2O)nn1)N1CCNCC1]

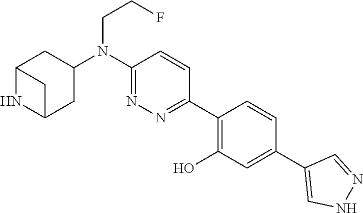 [SMILES: Oc1cc(-c2cn[nH]c2)ccc1-c1ccc(N(CCF)C2CC3CC(C2)N3)nn1]

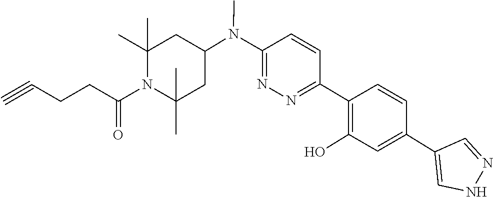

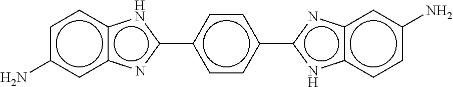

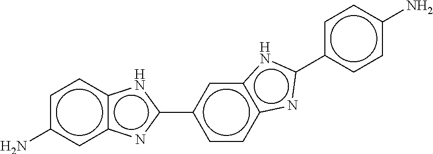

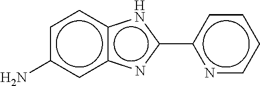

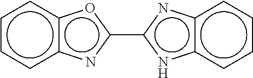

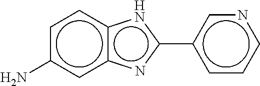

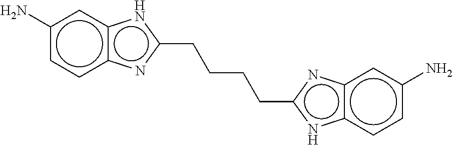

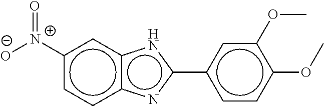

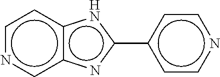

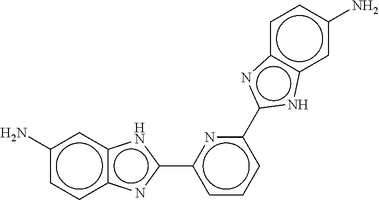

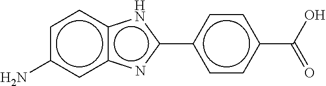

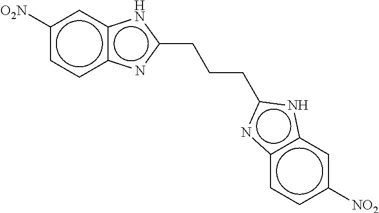

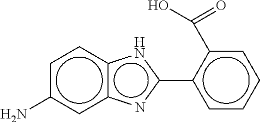

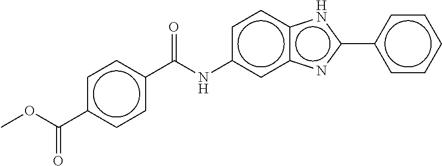

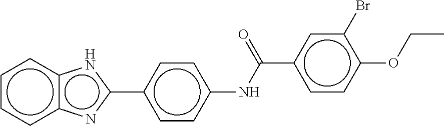

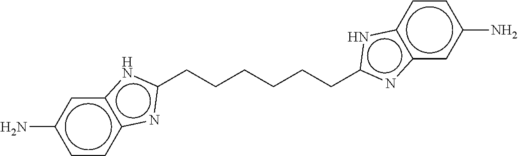

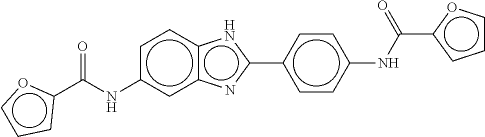

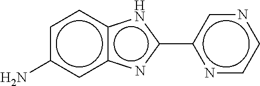

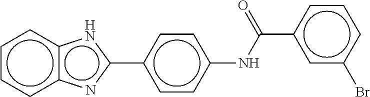

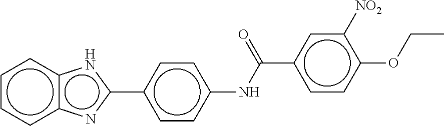

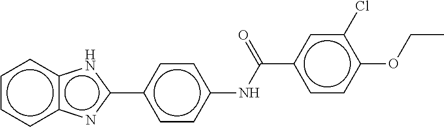

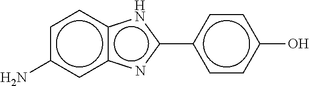

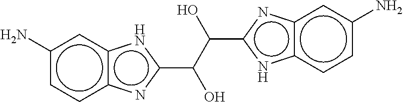

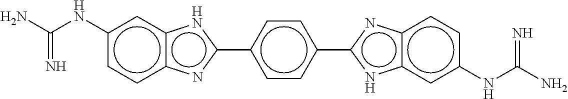

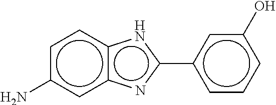

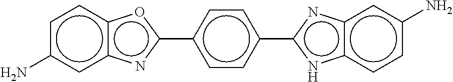

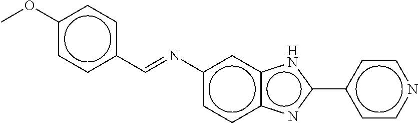

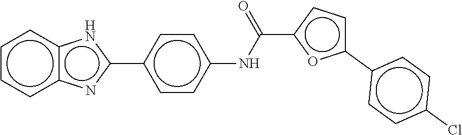

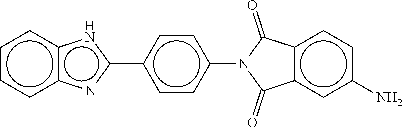

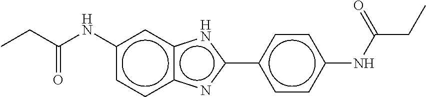

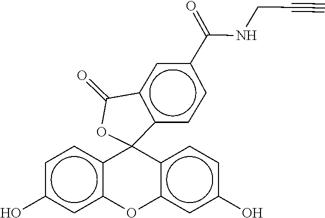

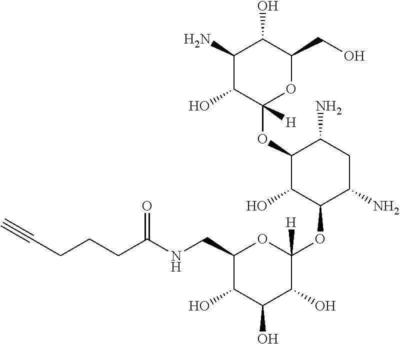 [SMILES: C#CCCCC(=O)NC[C@H]1O[C@H](O[C@H]2[C@H](O)[C@@H](O[C@H]3O[C@H](CO)[C@@H](O)[C@H](N)[C@H]3O)[C@H](N)C[C@@H]2N)[C@H](O)[C@@H](O)[C@@H]1O]

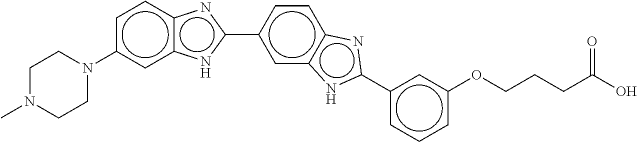

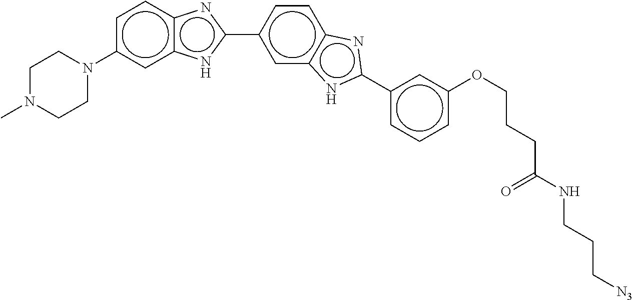

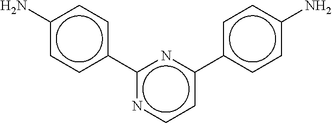

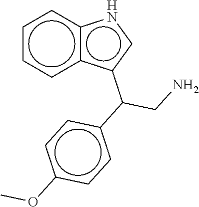

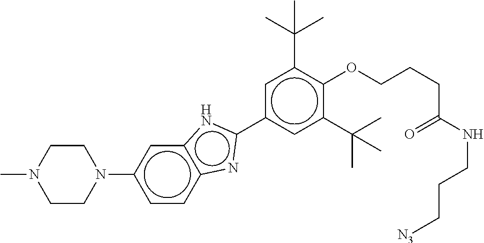

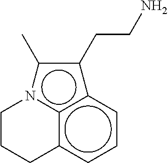

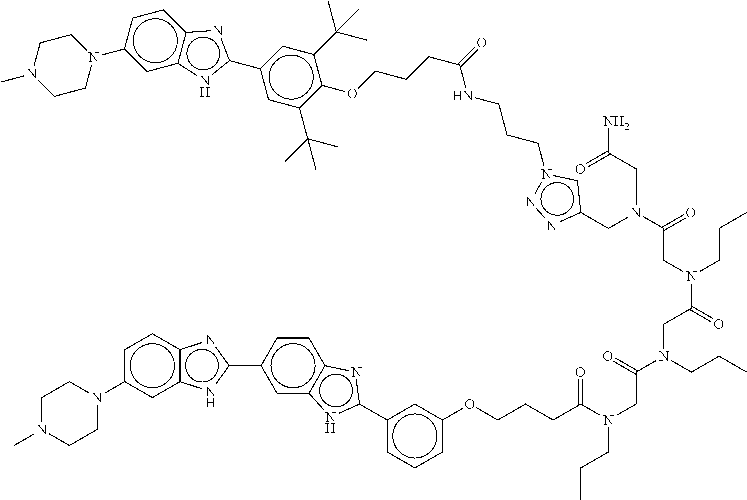

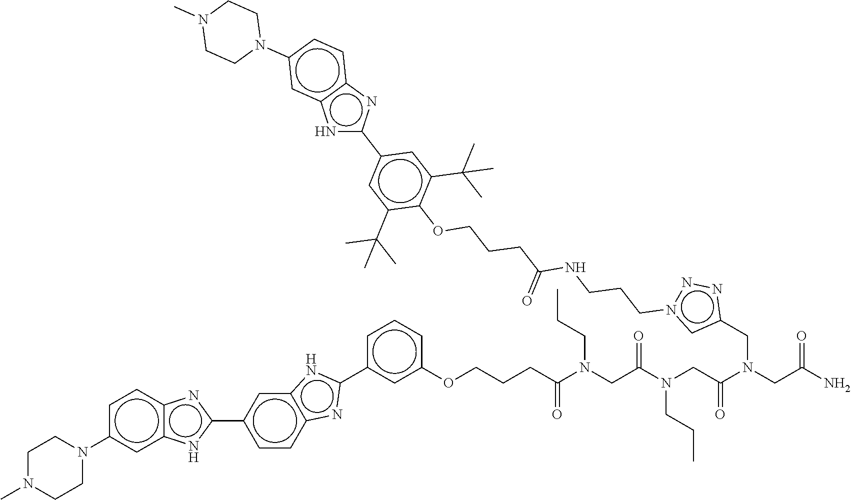

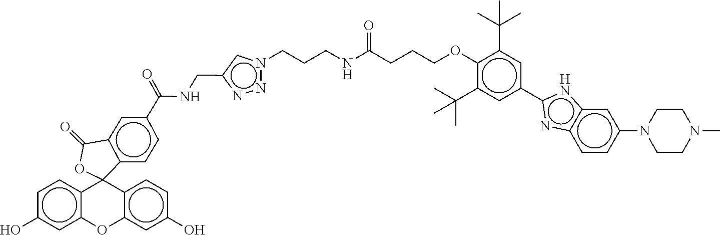

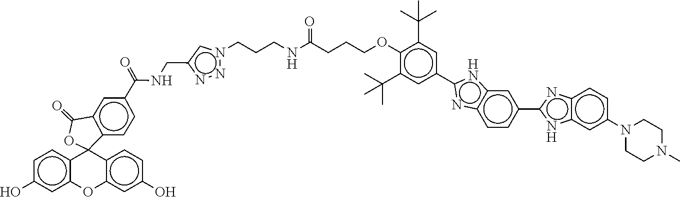

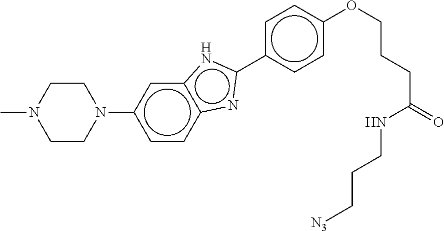

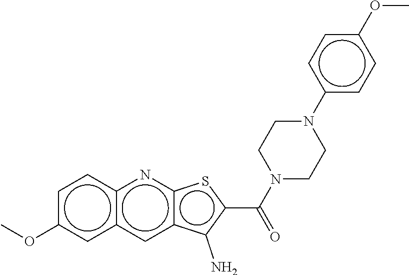

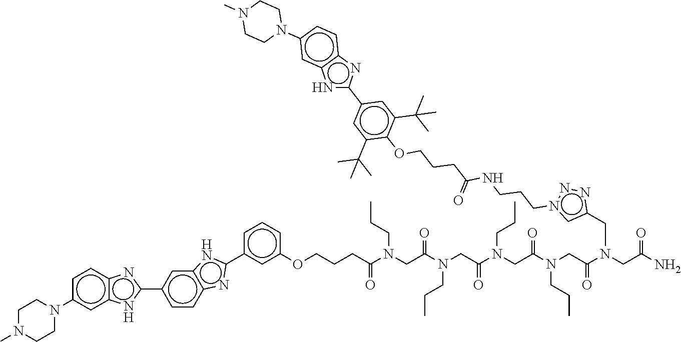

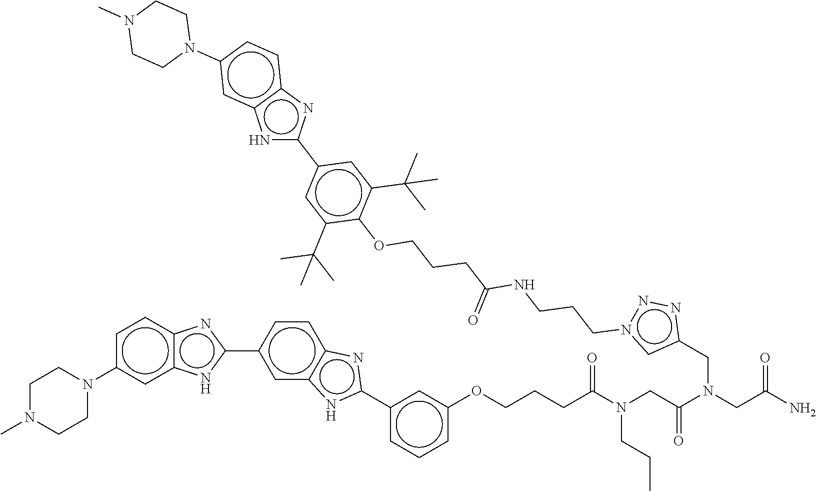

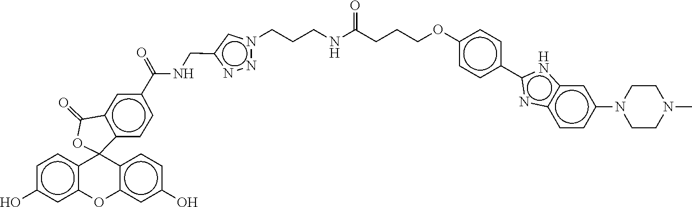

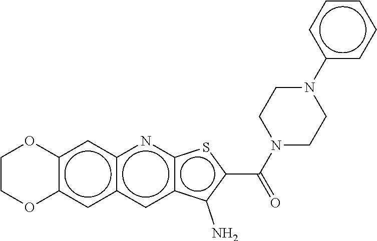

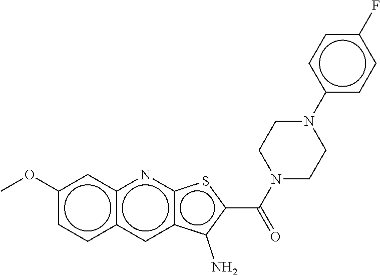

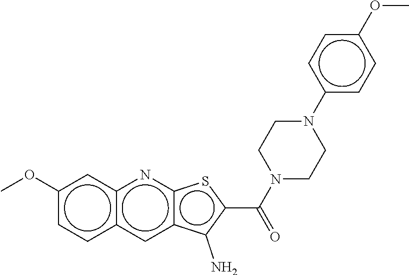

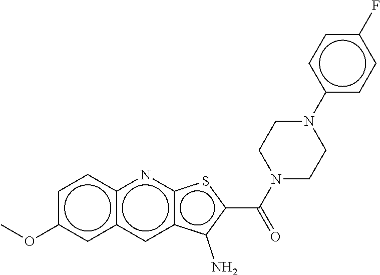

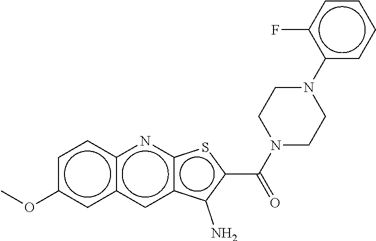

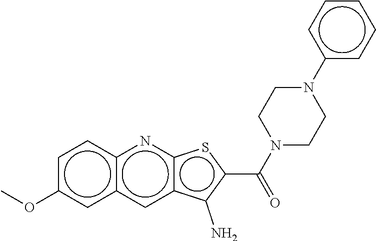

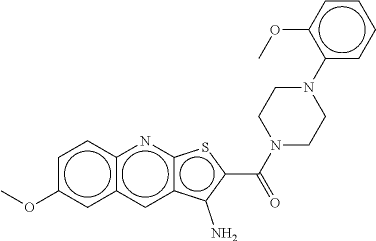

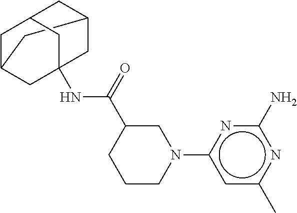

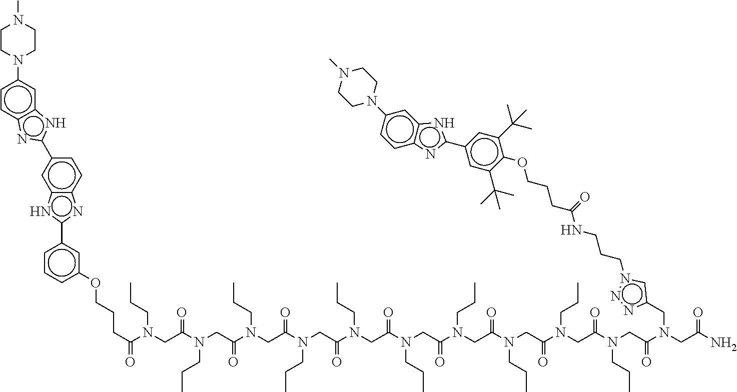

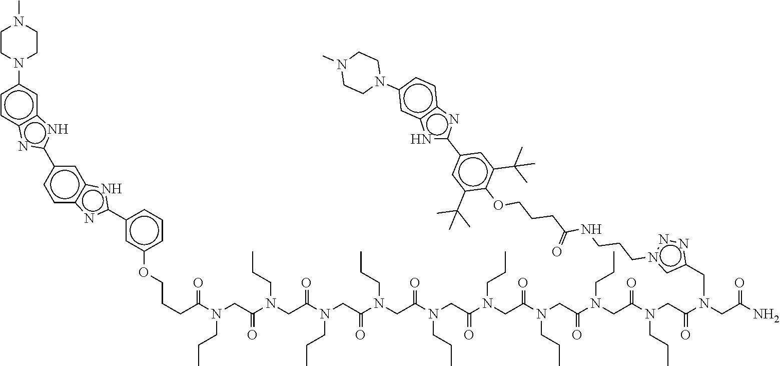

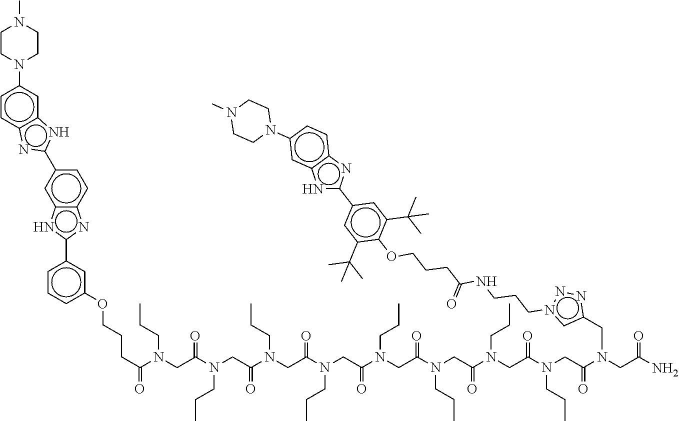

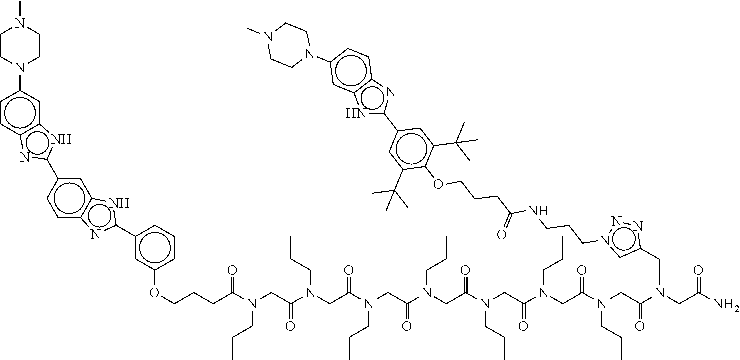

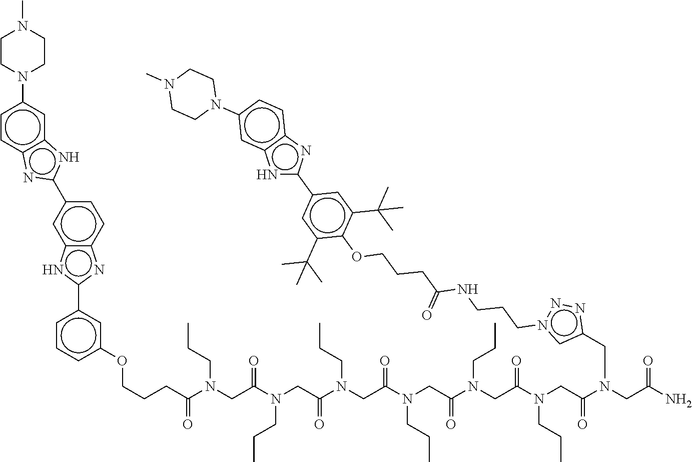

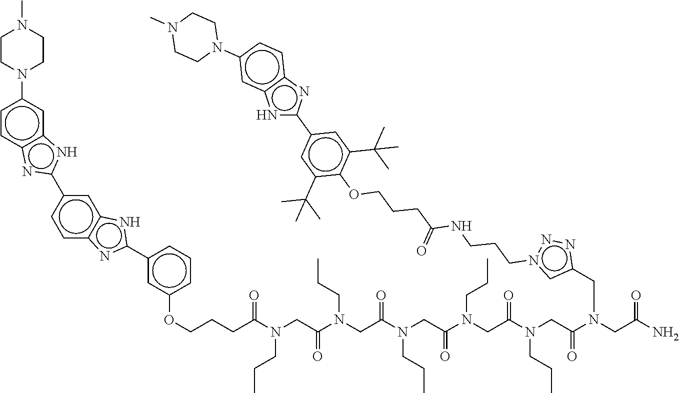

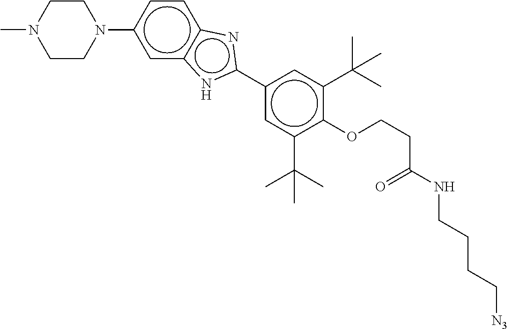

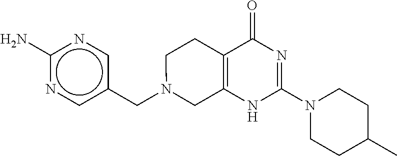

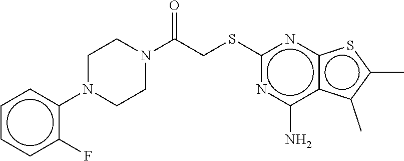

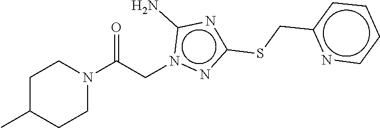

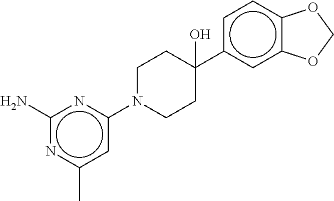

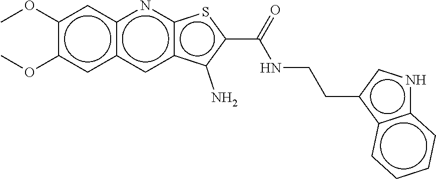

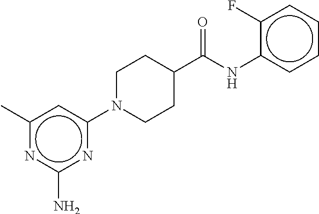

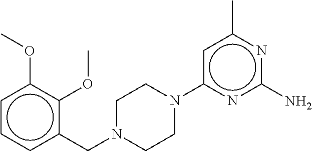

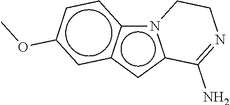

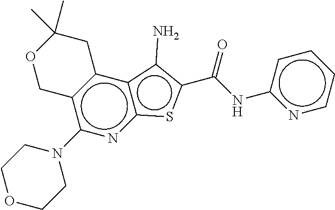

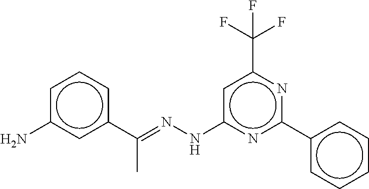

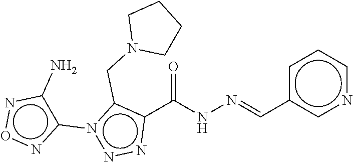

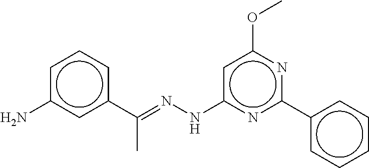

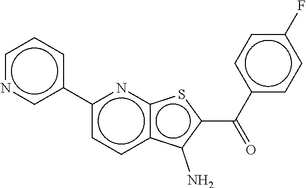

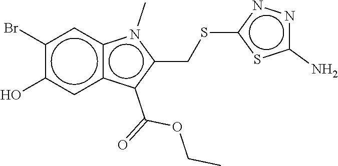

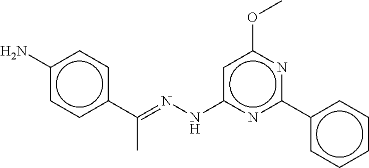

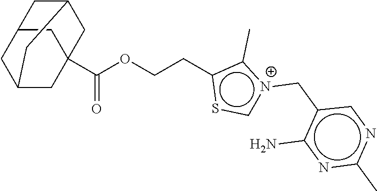

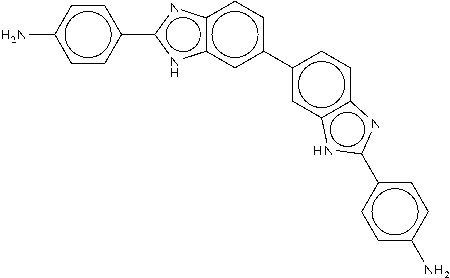

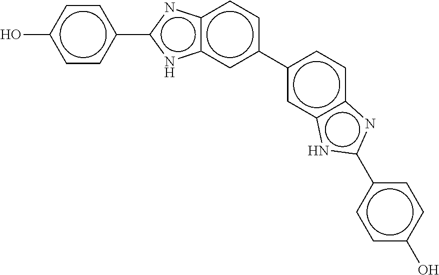

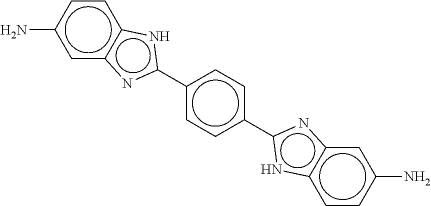

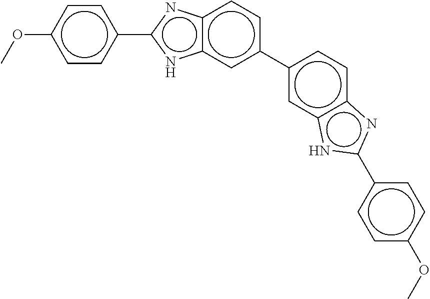

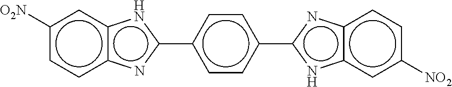

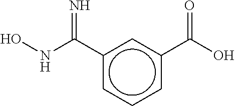

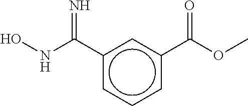

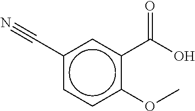

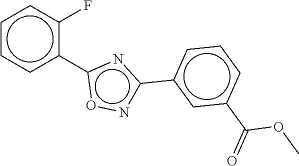

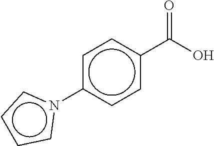

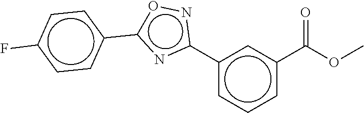

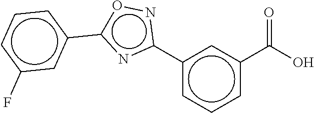

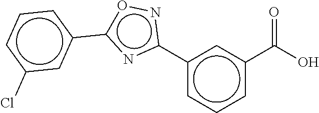

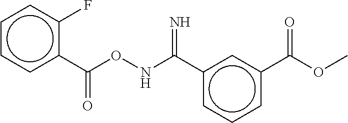

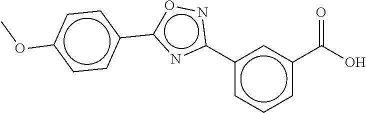

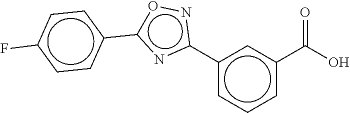

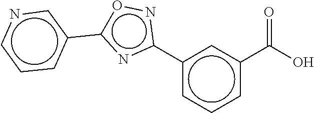

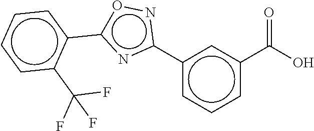

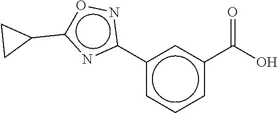

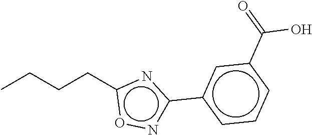

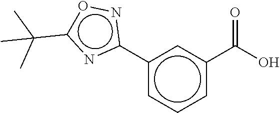

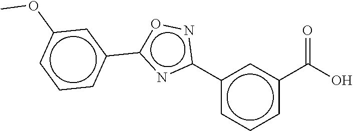

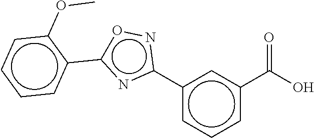

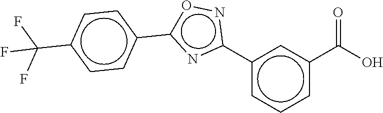

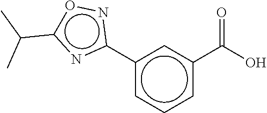

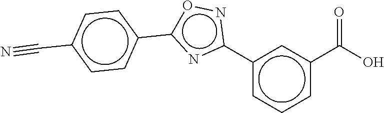

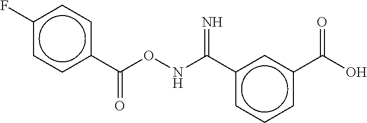

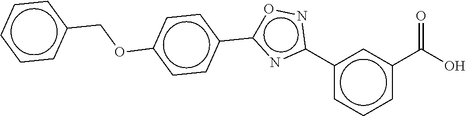

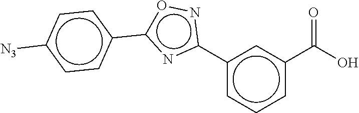

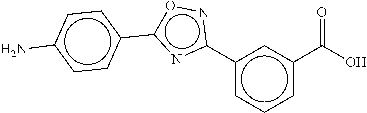

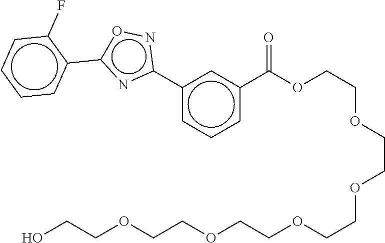

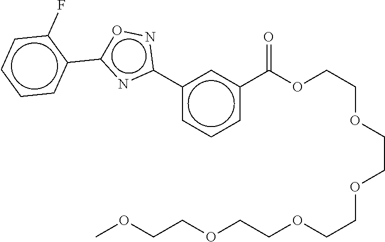

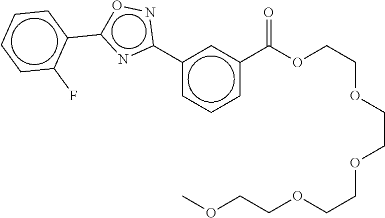

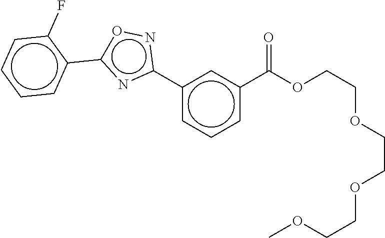

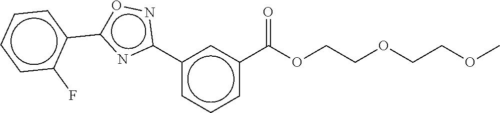

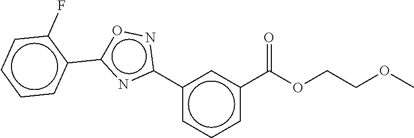

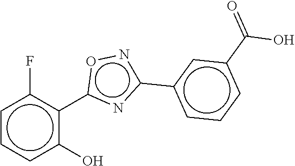

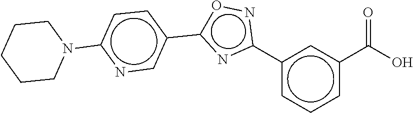

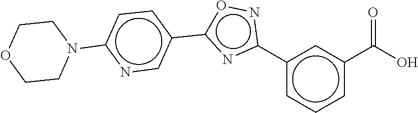

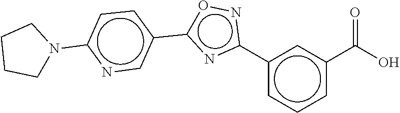

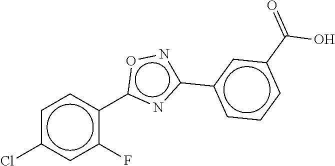

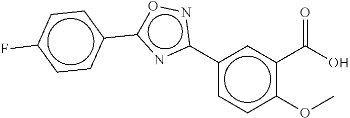

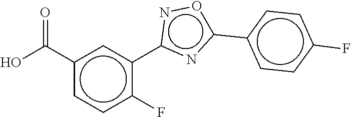

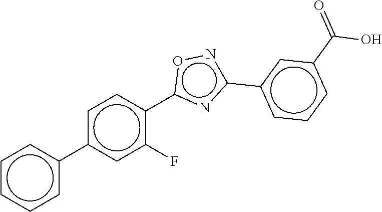

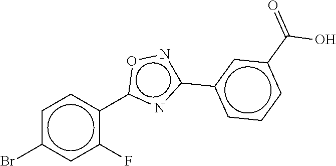

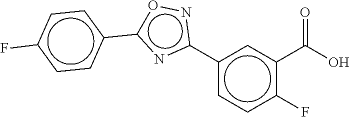

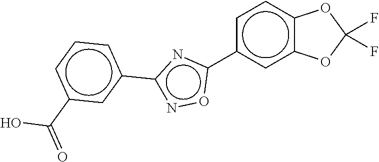

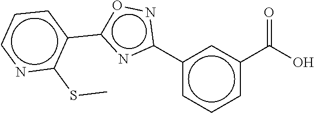

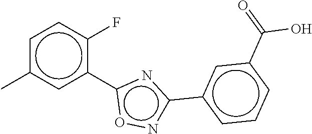

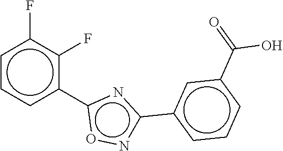

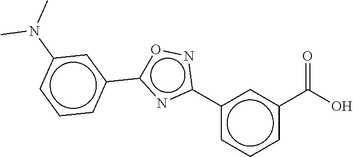

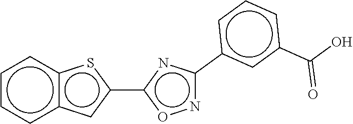

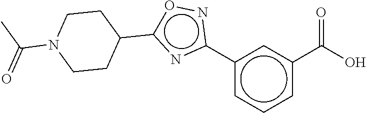

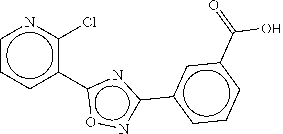

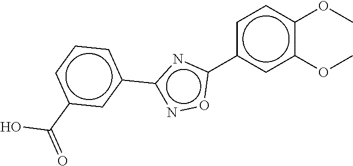

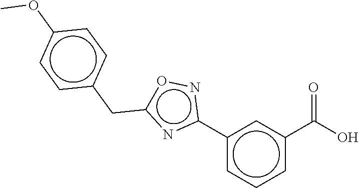

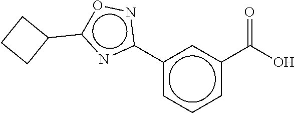

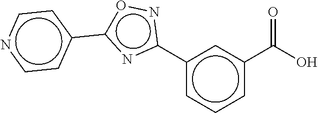

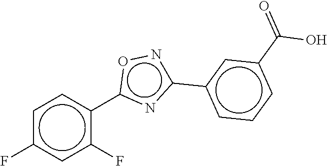

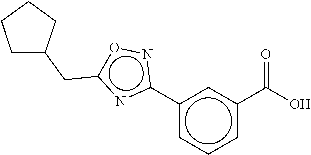

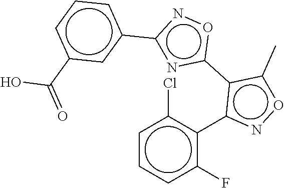

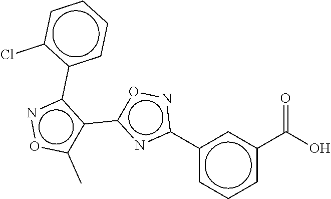

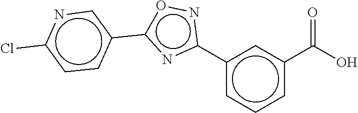

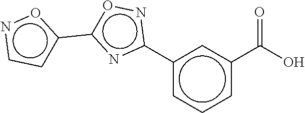

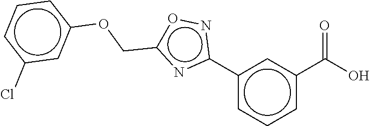

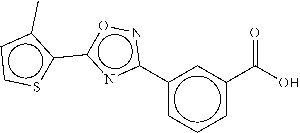

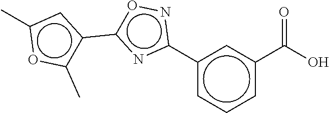

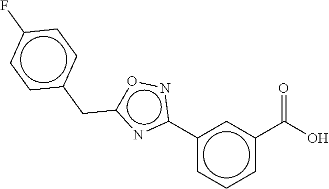

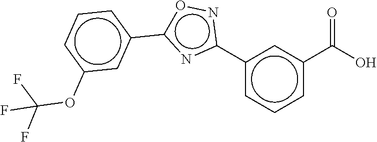

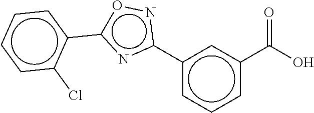

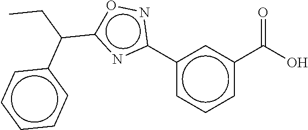

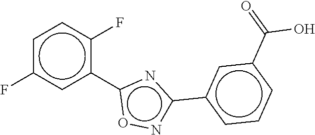

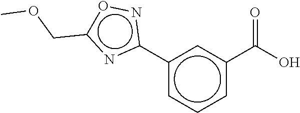

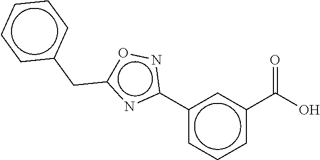

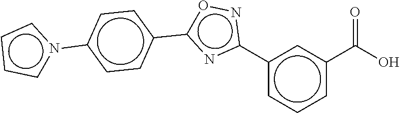

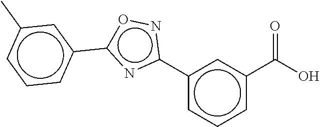

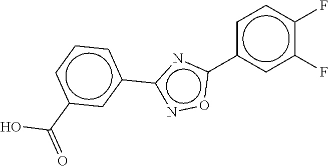

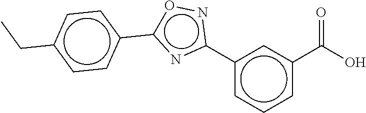

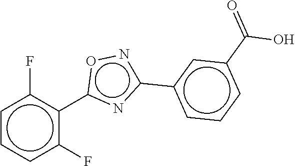

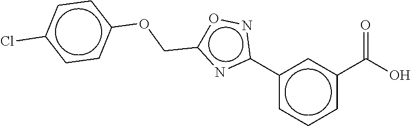

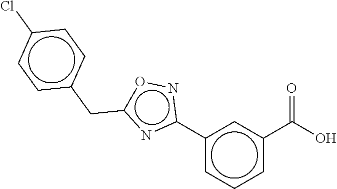

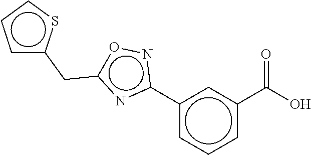

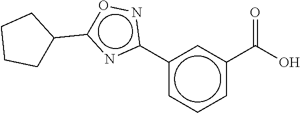

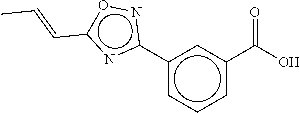

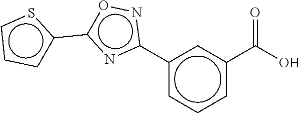

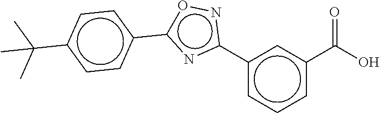

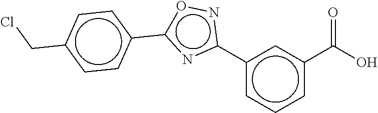

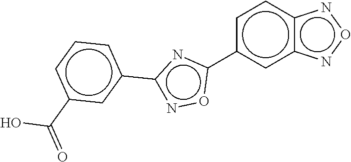

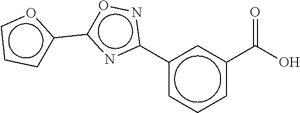

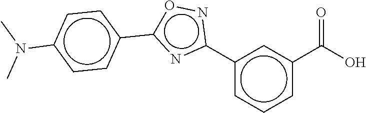

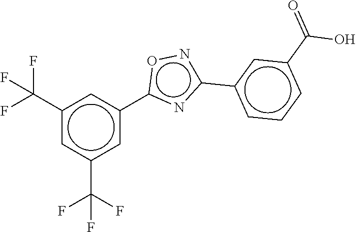

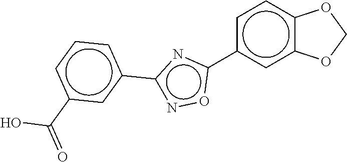

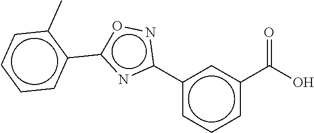

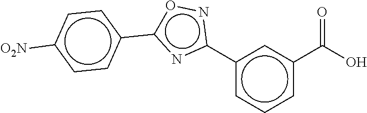

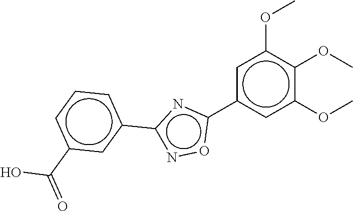

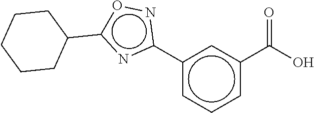

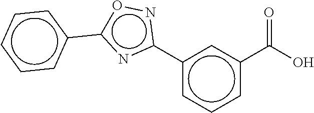

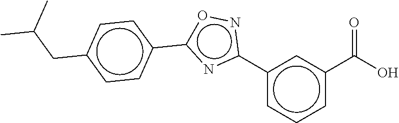

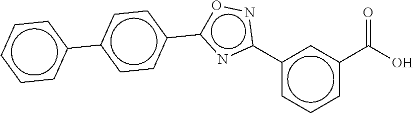

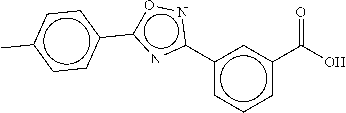

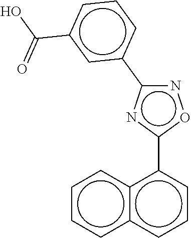

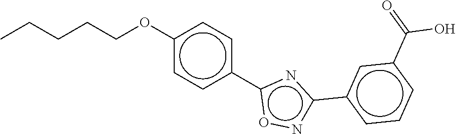

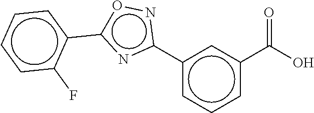

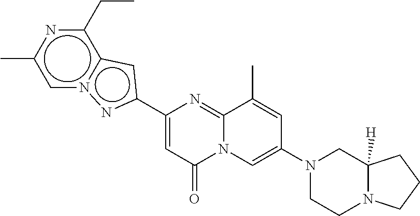

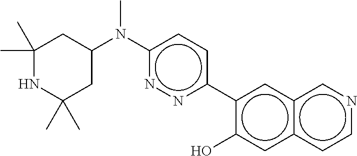

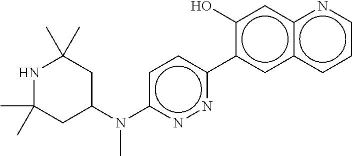

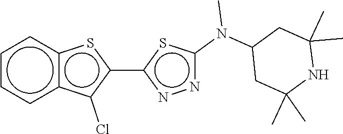

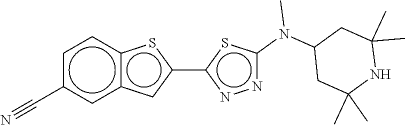

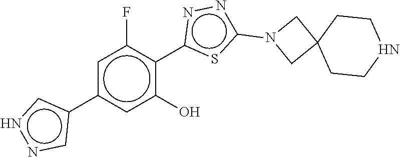

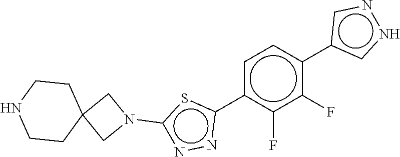

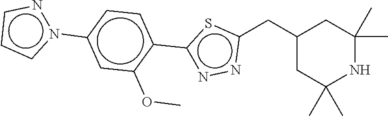

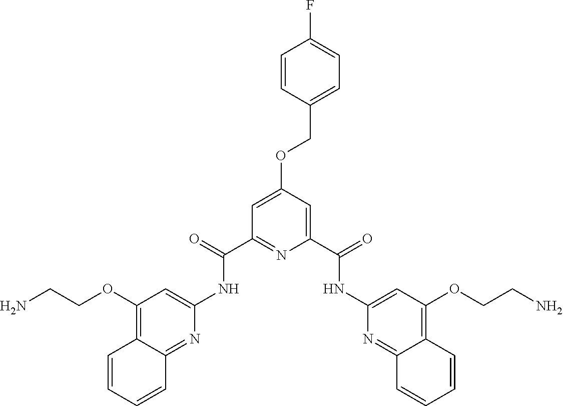 [SMILES: NCCOc1cc(NC(=O)c2cc(OCc3ccc(F)cc3)cc(C(=O)Nc3cc(OCCN)c4ccccc4n3)n2)nc2ccccc12]

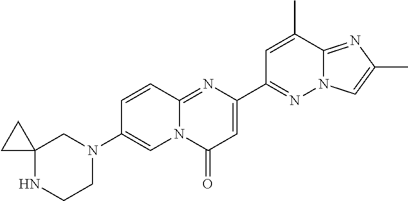 [SMILES: Cc1cn2nc(-c3cc(=O)n4cc(N5CCNC6(CC6)C5)ccc4n3)cc(C)c2n1]

1, (RG7916)
risdiplam

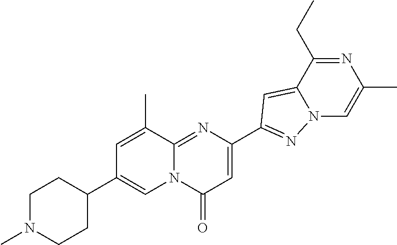

2, (RG7800)

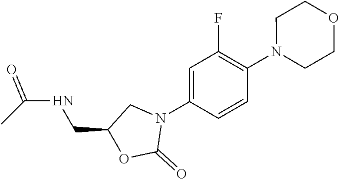 [SMILES: CC(=O)NC[C@H]1CN(c2ccc(N3CCOCC3)c(F)c2)C(=O)O1]

Linezolid
Warner, K. D., et al., Nat Rev Drug Discov.
2018, 17(8), 547-558; hereby incorporated by
reference.

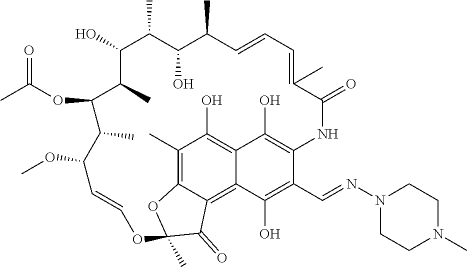 [SMILES: CO[C@H]1/C=C/O[C@@]2(C)Oc3c(C)c(O)c4c(O)c(c(/C=N/N5CCN(C)CC5)c(O)c4c3C2=O)NC(=O)/C(C)=C/C=C/[C@H](C)[C@H](O)[C@@H](C)[C@@H](O)[C@@H](C)[C@H](OC(C)=O)[C@@H]1C]

Warner, K. D. Nat Rev Drug Discov. 2018.

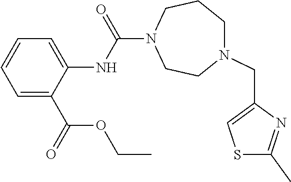 [SMILES: CCOC(=O)c1ccccc1NC(=O)N1CCCN(Cc2csc(C)n2)CC1]

Warner, K. D. Nat Rev Drug Discov. 2018.

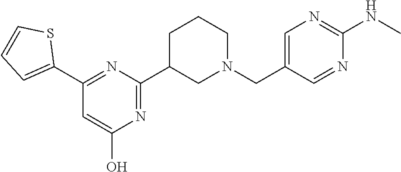 [SMILES: CNc1ncc(CN2CCCC(c3nc(O)cc(-c4cccs4)n3)C2)cn1]

Warner, K. D. Nat Rev Drug Discov. 2018.

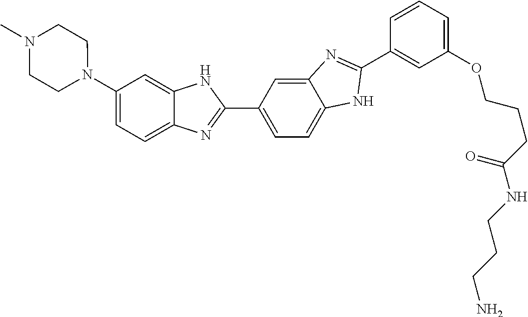 [SMILES: CN1CCN(c2ccc3nc(-c4ccc5[nH]c(-c6cccc(OCCCC(=O)NCCCN)c6)nc5c4)[nH]c3c2)CC1]

Warner, K. D. Nat Rev Drug Discov. 2018.

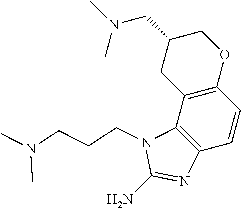 [SMILES: CN(C)CCCn1c(N)nc2ccc3c(c21)C[C@H](CN(C)C)CO3]

Warner, K. D. Nat Rev Drug Discov. 2018.

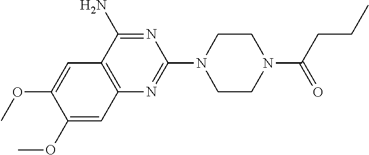 [SMILES: CCCC(=O)N1CCN(c2nc(N)c3cc(OC)c(OC)cc3n2)CC1]

Warner, K. D. Nat Rev Drug Discov. 2018.

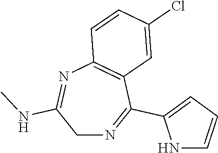 [SMILES: CNC1=Nc2ccc(Cl)cc2C(c2ccc[nH]2)=NC1]

Warner, K. D. Nat Rev Drug Discov. 2018.

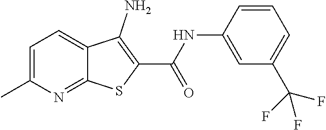 [SMILES: Cc1ccc2c(N)c(C(=O)Nc3cccc(C(F)(F)F)c3)sc2n1]

Warner, K. D. Nat Rev Drug Discov. 2018.

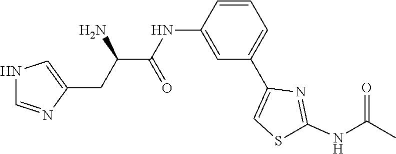 [SMILES: CC(=O)Nc1nc(-c2cccc(NC(=O)[C@H](N)Cc3c[nH]cn3)c2)cs1]

Warner, K. D. Nat Rev Drug Discov. 2018.

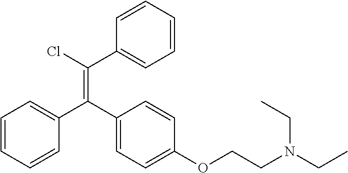 [SMILES: CCN(CC)CCOc1ccc(/C(=C(/Cl)c2ccccc2)c2ccccc2)cc1]

Warner, K. D. Nat Rev Drug Discov. 2018.

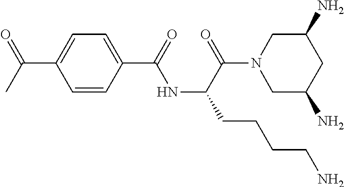 [SMILES: CC(=O)c1ccc(C(=O)N[C@@H](CCCCN)C(=O)N2C[C@H](N)C[C@H](N)C2)cc1]

Warner, K. D. Nat Rev Drug Discov. 2018.

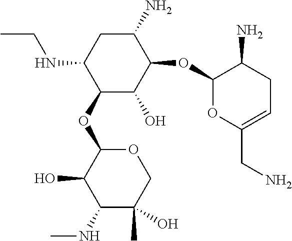 [SMILES: CCN[C@@H]1C[C@H](N)[C@@H](O[C@@H]2OC(CN)=CC[C@@H]2N)[C@H](O)[C@H]1O[C@H]1OC[C@](C)(O)[C@H](NC)[C@H]1O]

Warner, K. D. Nat Rev Drug Discov. 2018.

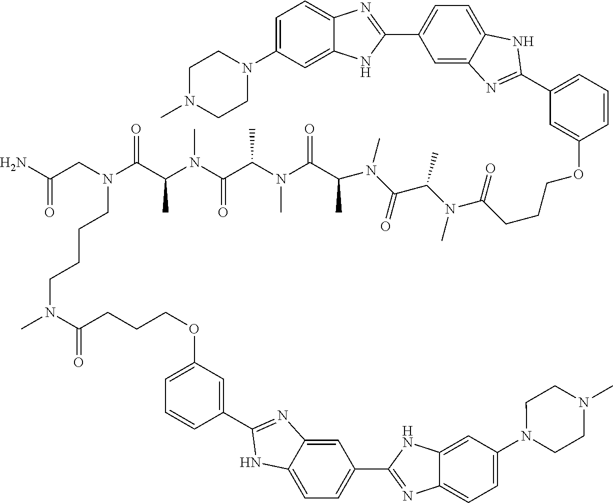

Warner, K. D. Nat Rev Drug Discov. 2018.

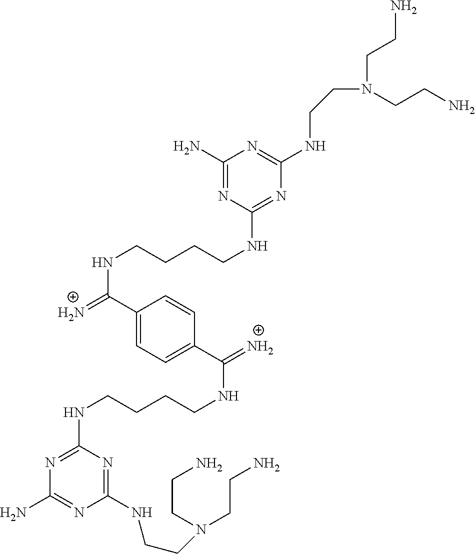 [SMILES: NCCN(CCN)CCNc1nc(N)nc(NCCCCNC(=[NH2+])c2ccc(C(=[NH2+])NCCCCNc3nc(N)nc(NCCN(CCN)CCN)n3)cc2)n1]

Warner, K. D. Nat Rev Drug Discov. 2018.

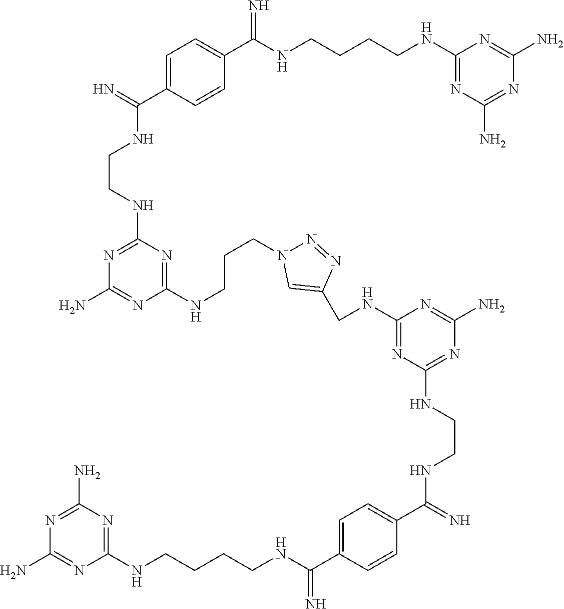 [SMILES: N=C(NCCCCNc1nc(N)nc(N)n1)c1ccc(C(=N)NCCNc2nc(N)nc(NCCCn3cc(CNc4nc(N)nc(NCCNC(=N)c5ccc(C(=N)NCCCCNc6nc(N)nc(N)n6)cc5)n4)nn3)n2)cc1]

Warner, K. D. Nat Rev Drug Discov. 2018.

### Decay Factors and RNA-Binding Proteins (RBPs)

In one aspect the disclosure provides a compound of Formula A:

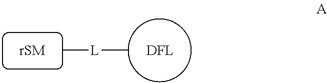

or a pharmaceutically acceptable salt thereof, wherein: rSM is an RNA-binding small molecule that binds to a target RNA transcript; DFL is a Decay Factor-recruiting Ligand; and L is a bivalent linker group that covalently connects the rSM to the DFL; wherein the DFL binds to or recruits a decay factor.

In one aspect, the disclosure provides a composition comprising an RNA binder that binds to a target RNA transcript and a Decay Factor-recruiting Ligand (DFL), wherein the DFL binds to or recruits a decay factor.

A decay factor as provided herein is any protein, polypeptide or biological molecule present in a cell that when brought in the proximity of a target RNA modulates that RNA. Modulating an RNA as provided herein includes, destabilizing the RNA, stabilizing the RNA, degrading the RNA, or acting on the RNA in any other capacity. Decay factors include any protein that interferes with the stability and/or activity of the RNA. In some embodiments, the decay factor is an RNA destabilizing protein, a nuclease, or an RNA-binding protein. It should be appreciated that nucleases and RNA-binding proteins are not mutually exclusive and that, for instance, some RNA-binding proteins also have nuclease activity. In some embodiments, the present disclosure provides a bifunctional compound or composition that effects recruitment to a target RNA to a nuclease capable of degrading the target RNA, or to an RNA-binding protein (RBP) that destabilizes the target RNA towards degradation by any of a cell's or tissue's mechanisms of RNA degradation.

In one aspect, the invention provides compounds and compositions that act on RNA without the need to induce RBPs. It should be appreciated that some RBPs need to be induced, dimerized or otherwise modified to be active. For instance, RNase L needs to be induced to dimerize and be active. Activating RBPs often is not a desired process as it results in the induction of unwanted elements. For instance, some RBPs are activated by inducing an immune response. In addition, RBPs often cannot be activated in cells in which activation is desirable. As disclosed herein, in some embodiments, the invention provides compounds and compositions that act on RNA without the need to induce RBPs. In some embodiments, the RBP is not RNase L.

In some embodiments, the decay factor (e.g., nuclease or RBP) can modulate the target RNA while the decay factor is at endogenous levels. In some embodiments, the RBP does not need to be induced to be active. In some embodiments, the RBP does not need to dimerize to be active. In some embodiments the RBP is not RNase L.

In some embodiments the RBP or decay factor does not have any nuclease, or other RNA degrading or destabilizing activity, but can bind or recruit a second protein or multi-component complex that has nuclease, degrading and/or RNA destabilizing activity.

In one aspect, the present disclosure provides a bifunctional compound or composition that effects recruitment of a target RNA to a decay factor, wherein the decay factor is present at endogenous levels. In some embodiments, the present disclosure provides a bifunctional compound or composition that effects recruitment of a target RNA to a nuclease capable of degrading the target RNA, wherein the nuclease is present at endogenous levels, or to an RNA-binding protein (RBP) that destabilizes the target RNA towards degradation by any of a cell's or tissue's mechanisms of RNA degradation, wherein the RBP that destabilizes the target RNA is present at endogenous levels.

In some embodiments, the DFL binds or attracts a complex of proteins that can degrade or otherwise modulate the RNA function (e.g., the availability for protein translation). In some embodiments, the protein complex is the CCR4-NOT (Carbon Catabolite Repression-Negative On TATA-less) complex.

### CCR4-NOT Complex

In some embodiments, the DFL binds or attracts a complex of proteins that can degrade or otherwise modulate the RNA function. In some embodiments, the DFL binds the protein complex. In some embodiments, the DFL binds one or more RBPs that are part of the protein complex. Binding of one or more RBPs is expected to bring the complete protein complex in proximity to the target RNA. In some embodiments, the DFL binds the CCR4-NOT (Carbon Catabolite Repression-Negative On TATA-less) complex, or an RBP that is a member of the CCR4-NOT complex. The CCR4-NOT complex is a large and highly conserved multifunctional assembly of proteins involved in different aspects of mRNA metabolism. Without wishing to be bound by theory, it is believed that the CCR4-NOT complex plays a role in deadenylation-dependent mRNA turnover. RBPs that are part of the CCR4-NOT complex include CNOT1, CNOT2, CNOT3, CNOT6, CNOT6L, CNOT7, CNOT8, CNOT9, CNOT10 and CNOT11. The function of the CCR4-NOT complex and each of the RBPs that make up the complex is discussed for instance in Shirai et al. Multifunctional roles of the mammalian CCR4-NOT complex in physiological phenomena, Frontiers in Genetics, 2014, 5, Article 286, which is incorporated by reference.

In some embodiments, the RBP is one of those listed in Table 1B. In some embodiments, the RBP is CNOT2, CNOT7, DDX6, YTHDF2, ZFP36, DCP1A, ZC3H12A (Regnase-1), PARN, MARF, or IRE-1. In some embodiments, the DFL is a small molecule ligand of an RBP listed in Table 1B, such as CNOT2, CNOT7, DDX6, YTHDF2, ZFP36, DCP1A, ZC3H12A (Regnase-1), PARN, MARF, or IRE-1. In some embodiments, the RBP is CNOT2, CNOT7 or YTHDF2.

### CNOT2

In some embodiments, the RBP is CNOT2. In some embodiments, a disclosed compound or composition comprises a small molecule CNOT2 ligand as the DFL. CNOT2 is a member of the CCR4-NOT complex. Without wishing to be bound by theory, it is believed that CNOT2 interfaces with other RBPs (e.g., RBPs of the CCR4-NOT complex) to result in deadenylation of the target RNA. CNOT2 is widely expressed in the human body.

### CNOT7

In some embodiments, the RBP is CNOT7. In some embodiments, a disclosed compound or composition comprises a small molecule CNOT7 ligand as the DFL. CNOT7 is a member of the CCR4-NOT complex. Without wishing to be bound by theory, it is believed that CNOT7 acts as exonuclease. It is thought to either directly, or in conjunction with other members of the CCR4-NOT complex, induce degradation of the target RNA (e.g., through deadenylation). CNOT7 is widely expressed in the human body.

### CNOT6

In some embodiments, the RBP is CNOT6. In some embodiments, a disclosed compound or composition comprises a small molecule CNOT6 ligand as the DFL. Without wishing to be bound by theory, it is believed that CNOT6 is the mammalian homolog of the yeast CCR4 protein and is part of the mammalian CCR4-NOT complex. The CNOT6 protein has 3′-5′ exoribonuclease activity and exhibits a preference for the removal of A residues from the mRNA 3′-polyA tail. Without wishing to be bound by theory, it is believed that CNOT6 exhibits a) 3′-5′ RNase activity that in turn causes rapid destabilization of mRNA, b) Ubiquitous expression, and c) broad substrate specificity.

In some embodiments, the RBP is CNOT2, CNOT7, YTHDF2, DDX6, ZFP36, DCP1A, ZC3H12A (Regnase-1), PARN, MARF, or IRE-1.

### YTHDF2

YTHDF2 stands for YTH N6-Methyladenosine RNA Binding Protein 2. In some embodiments, the RBP is YTHDF2. In some embodiments, a disclosed compound or composition comprises a small molecule YTHDF2 ligand as the DFL. Without wishing to be bound by theory, YTHDF2 is thought to specifically recognize and bind N6-methyladenosine (m6A)-containing RNAs, thereby regulating the RNAs stability. It is also though to act as a regulator of mRNA stability by promoting degradation of m6A-containing mRNAs via interaction with the CCR4-NOT and ribonuclease P/MRP complexes, depending on the context.

### DDX6

In some embodiments, the RBP is DDX6. In some embodiments, a disclosed compound or composition comprises a small molecule DDX6 ligand as the DFL. DDX6 is a member of the DEAD box protein family. Without wishing to be bound by theory, DDX6 is thought to be an RNA helicase and is found in P-bodies and stress granules, and functions in translation suppression and mRNA degradation.

### ZFP36

In some embodiments, the RBP is ZFP36. In some embodiments, a disclosed compound or composition comprises a small molecule ZFP36 ligand as the DFL. Without wishing to be bound by theory, it is believed that ZFP36 represses mRNA target abundance and translation, notably through novel AU-rich sites in coding sequences. ZFP36 binds to AU-rich elements (AREs) in the 3′-untranslated regions (UTRs) of the mRNAs of some cytokines and promotes their degradation. For example, TTP is a component of a negative feedback loop that interferes with TNF-alpha production by destabilizing its mRNA.

### DCP1, DCP1A or DCP2

In some embodiments, the RBP is DCP1, DCP1A or DCP2. In some embodiments, a disclosed compound or composition comprises a small molecule DCP2 ligand as the DFL. Without wishing to be bound by theory, it is believed that DCP2 is the catalytic subunit of the DCP1-DCP2 complex of proteins. By using a DFL against either DCP1 (DCP1A) or DCP2, the DCP1-DCP2 complex can be brought in the proximity of the target RNA. DCP2 catalyzes removal of the mRNA 5′-cap, which inhibits mRNA translation and promotes mRNA degradation by the 5′-3′ ribonuclease, Xrn1. The DCP1-DCP2 complex functions on all mRNAs in vivo. Without wishing to be bound by theory, it is believed that DCP1-DCP2 provides a) Rapid mRNA destabilization via decapping, b) ubiquitous expression, and c) broad substrate specificity.

### ZC3H12A (Regnase-1)

In some embodiments, the RBP is ZC3H12A (Regnase-1). In some embodiments, a disclosed compound or composition comprises a small molecule ZC3H12A (Regnase-1) ligand as the DFL. Without wishing to be bound by theory, it is believed that ZC3H12A (Regnase-1) functions as an endoribonuclease involved in mRNA decay.

### PARN

In some embodiments, the RBP is PARN. In some embodiments, a disclosed compound or composition comprises a small molecule PARN ligand. Without wishing to be bound by theory, it is believed that PARN protein is a 3′-exoribonuclease, with similarity to the RNase D family of 3′-exonucleases. It prefers poly(A) as the substrate, hence, efficiently degrades poly(A) tails of mRNAs. Exonucleolytic degradation of the poly(A) tail is often the first step in the decay of eukaryotic mRNAs. PARN is also involved in silencing of certain maternal mRNAs during oocyte maturation and early embryonic development, as well as in nonsense-mediated decay (NMD) of mRNAs that contain premature stop codons.

### MARF1

MARF1 is an abbreviation for Meiosis arrest factor 1 and is an mRNA destabilization protein. In some embodiments, the RBP is MARF1. In some embodiments, a disclosed compound or composition comprises a small molecule MARF1 ligand. Without wishing to be bound by theory, it is believed that MARF1 interactions with the Dcp1-Dcp2 proteins, which facilitate mRNA decay. It is further believed that MARF1 also has an intrinsic endonuclease activity. Artificially tethering of MARF1 to a reporter mRNA causes mRNA decay, and this activity is not dependent on its interaction with Dcp1-Dcp2. Without wishing to be bound by theory, it is believed that MARF1 provides a) mRNA destabilization via ribonuclease activity and Dcp1-Dcp2 recruitment, and b) broad substrate specificity as demonstrated by genetic tethering.

### IRE1

In some embodiments, the RBP is IRE1. In some embodiments, a disclosed compound or composition comprises a small molecule IRE1 ligand as the DFL. Without wishing to be bound by theory, it is believed that IRE/ERN1 is an Endoplasmic Reticulum (ER)-associated protein, which functions downstream of the Unfolded Protein Response (UPR). IRE1 is a transmembrane protein present as monomers on the ER-membrane. Upon detection of ER stress, the protein undergoes phosphorylation, which causes oligomerization and activation of its RNase activity. While the primary target of IRE1 RNase activity is XBP1 mRNA, some reports suggest that IRE1 cleaves several mRNAs. It is further believed that IRE1 shows a) RNase activity and b) Ubiquitous expression.

In some embodiments, the RBP is SMG7, KSRP, or SMG6.

### SMG7

In some embodiments, the RBP is SMG7. In some embodiments, a disclosed compound or composition comprises a small molecule SMG7 ligand as the DFL. SMG7 is a part of the nonsense-mediated decay (NMD) pathway and is recruited to a faulty mRNA via its interaction with Upf1. In genetic tethering experiments SMG7 was shown to function epistatic to the upstream mRNA surveillance proteins and caused severe reduction in reporter mRNA levels via the deadenylation dependent mRNA decay pathway. Without wishing to be bound by theory, it is believed that SMG7 shows a) broad substrate specificity, b) no activation step (activity is restricted by mRNA recruitment), c) no directionality bias for activity, (it can be tethered to the target mRNA 5′- or 3′-end for mRNA destabilization), and d) it is believed to exhibit high to moderate expression in most cell and tissue types.

### KSRP

In some embodiments, the RBP is KSRP. In some embodiments, a disclosed compound or composition comprises a small molecule KSRP ligand as the DFL. KSRP (also known as KHSRP) is a KH-domain-containing protein that functions in mRNA degradation of specific mRNA targets in vivo. KSRP is an AU-rich element binding protein. Without wishing to be bound by theory, it is believed that, upon binding to the AU-rich element (ARE), it promotes mRNA degradation via deadenylation and a 3′-5′ decay pathway. It is further believed that KSRP exhibits a) broad substrate specificity, b) no activation step (activity is restricted by mRNA recruitment, c) rapid kinetics for mRNA decay, and d) ubiquitous expression in cell and tissue types.

### SMG6

In some embodiments, the RBP is SMG6. In some embodiments, a disclosed compound or composition comprises a small molecule SMG6 ligand as the DFL. Without wishing to be bound by theory, it is believed that SMG6 is a PIN-domain containing endonuclease associated with the NMD pathway. SMG6 is recruited to NMD substrate mRNAs via an activated Upf1 protein. Once recruited, SMG6 cleaves mRNA endonucleolytically. Experiments involving SMG6 tethering to mRNAs have revealed that it can cleave reporter mRNA but still requires assembly of the NMD-associated protein complex on the mRNA. Without wishing to be bound by theory, it is believed that SMG6 exhibits a) RNase activity, b) Ubiquitous expression, and c) broad substrate specificity.

In some embodiments, the RBP is selected from one of those in Table 1B, below. In some embodiments, the DFL is a ligand for an RBP listed in Table 1B.

TABLE 1B
Exemplary RNA-Binding Proteins (RBPs)

| Protein Name | UniProt |
|---|---|
| Angiogenin | ANGI_HUMAN |
| APE-1 | APEX1_HUMAN |
| ARD-1 | PP1R8_HUMAN |
| AUF1 | HNRPD_HUMAN |
| BRF1 | TF3B_HUMAN |
| C1D | C1D_HUMAN |
| CCR4-NOT complex | |
| CCR4 | CNOT6_HUMAN |
| CNOT1 | CNOT1_HUMAN |
| CNOT2 | CNOT2_HUMAN |
| CNOT3 | CNOT3_HUMAN |
| CNOT4 | CNOT4_HUMAN |
| CNOT6 | CNOT6_HUMAN |
| CNOT6l | CNO6L_HUMAN |
| CNOT8 | CNOT8_HUMAN |
| Cue2 | SMRCD_HUMAN |
| DBR1 | DBR1_HUMAN |
| DCP1A | DCP1A_HUMAN |
| DCP1B | DCP1B_HUMAN |
| DCP2 | DCP2_HUMAN |
| DDX6 (RCK) | DDX6_HUMAN |
| EXOSC1 | EXOS1_HUMAN |
| EXOSC10 | RRP6, EXOSX_HUMAN |
| EXOSC2 | EXOS2_HUMAN |
| EXOSC3 | EXOS3_HUMAN |
| EXOSC4 | EXOS4_HUMAN |
| EXOSC5 | EXOS5_HUMAN |
| EXOSC6 | EXOS6_HUMAN |
| EXOSC7 | EXOS7_HUMAN |
| EXOSC8 | EXOS8_HUMAN |
| EXOSC9 | EXOS9_HUMAN |
| Exosome complex | |
| HuR | ELAV1_HUMAN |
| IRE1 | ERN1_HUMAN |
| KSRP | FUBP2_HUMAN |
| LSM1 | LSM1_HUMAN |
| LSM2 | LSM2_HUMAN |
| LSM3 | LSM3_HUMAN |
| LSM4 | LSM4_HUMAN |
| LSM5 | LSM5_HUMAN |
| LSM6 | LSM6_HUMAN |
| LSM7 | LSM7_HUMAN |
| LSM8 | LSM8_HUMAN |
| MARF1 | MARF1_HUMAN |
| MPP6 | MPH6_HUMAN |
| NOB1 | NOB1_HUMAN |
| PAN2 | PAN2_HUMAN |
| PAN3 | PAN3_HUMAN |
| PAPD5 | PAPD5_HUMAN |
| PARN | PARN_HUMAN |
| PATL1 | PATL1_HUMAN |
| POLS | PAPD7_HUMAN |
| PP11 | ENDOU_HUMAN |
| RAT1 | XRN2_HUMAN |
| REG-1 (Regnase-1; | ZC12A_HUMAN |
| aka ZC3H12A) | |
| RNASEL | RN5A_HUMAN |
| RNASEN | RNC_HUMAN |
| RNY1 | S4R322_HUMAN |
| RRP44 | RRP44_HUMAN |
| SKI1-7 | MBTP1_HUMAN, SKIV2_HUMAN, |
| | TTC37_HUMAN, SOSB1_HUMAN, |
| | EXOS4_HUMAN, RENT1_HUMAN |
| SKIV2L2 | MTREX_HUMAN |
| SMG6 | EST1A_HUMAN |
| SMG7 | SMG7_HUMAN |
| Staufen1 | STAU1_HUMAN |
| SUPV3L1 | SUV3_HUMAN |
| Tristetraprolin | TTP_HUMAN |
| UPF1 | RENT1_HUMAN |
| UPF2 | RENT2_HUMAN |
| UPF3A, UPF3B | REN3A_HUMAN, REN3B_HUMAN |
| XRN1 | XRN1_HUMAN |
| XRN2 | XRN2_HUMAN |
| Y14 | Y14_HUMAN |
| YTHDF2 | YTHD2_HUMAN |
| ZCCHC3 | ZCHC3_HUMAN |
| ZFP36 | TTP_HUMAN |
| ZFP36L1 | TISB_HUMAN |
| ZFP36L2 | TISD_HUMAN |

### Decay Factor Ligands

In one aspect, the disclosure provides bifunctional compounds of Formula A wherein the compound includes a Decay Factor Ligand. Decay Factor Ligands are chemical moieties that can bind a decay factor, such as nuclease or RBP. Decay Factors Ligands are not so limited as long as they can bind the decay factor and/or allow the decay factor to act on the target RNA transcript.

In some embodiments, the DFL is a small molecule or pharmaceutically acceptable salt thereof. In some embodiments, the DFL has a molecular weight (MW) of 1000 or less. In some embodiments, the DFL has a MW of about 750 or less. In some embodiments, the DFL has a MW of about 600 or less. In some embodiments, the DFL has a MW of about 500 or less. In some embodiments, the DFL has a MW of between about 100 and about 1000. In some embodiments, the DFL has a MW of between about 150 and about 800, about 150 and about 600, about 150 and about 400, about 150 and about 350, about 200 and about 350, or between about 200 and about 450.

Methods to identify DFLs for RBPs of interest are well established (e.g. DNA-encoded library screening)

In some embodiments, the DFL is selected from one of those depicted in Table 1C, below.

TABLE 1C
Exemplary Decay Factor-recruiting Ligands (DFLs)

| Compound # | Structure |
|---|---|
| X-1 | 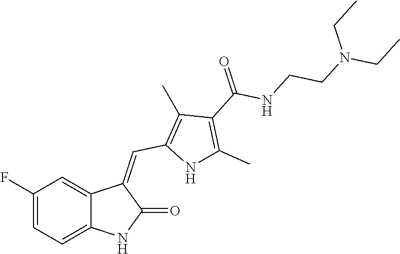 [SMILES: CCN(CC)CCNC(=O)c1c(C)[nH]c(/C=C2\C(=O)Nc3ccc(F)cc32)c1C] Jha, B. K. et al., J. Bio. Chem. 2011, 286, 26319-26326; hereby incorporated by reference. |
| X-2 | 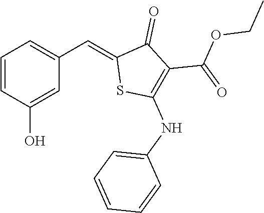 [SMILES: CCOC(=O)C1=C(Nc2ccccc2)S/C(=C\c2cccc(O)c2)C1=O] |
| X-3 | 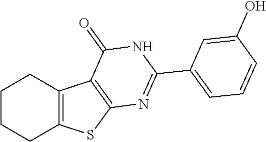 [SMILES: O=c1[nH]c(-c2cccc(O)c2)nc2sc3c(c12)CCCC3] Thakur, C.S. et al., Proc. Natl. Acad. Sci. USA 2007, 104, 9585-9590; hereby incorporated by reference. |
| X-4 | 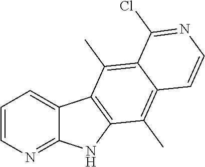 [SMILES: Cc1c2ccnc(Cl)c2c(C)c2c1[nH]c1ncccc12] NMDI1 Durand, S. et al., J. Cell Bio, 2007, 178, 1145- 1160; hereby incorporated by reference. |
| X-5 | 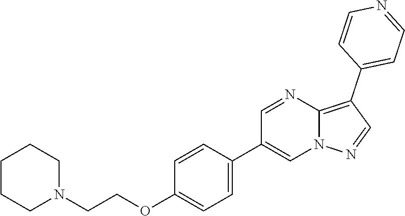 [SMILES: c1cc(-c2cnn3cc(-c4ccc(OCCN5CCCCC5)cc4)cnc23)ccn1] Cheruiyot A. et al., PLoS ONE 2018, 13(10), e0204978; hereby incorporated by reference. |
| X-6 | 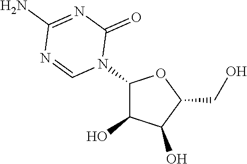 [SMILES: Nc1ncn([C@@H]2O[C@H](CO)[C@@H](O)[C@H]2O)c(=O)n1] Bhuvanagiri, M. et al., EMBO Mol Med 2014, 6, 1593-1609; hereby incorporated by reference. |
| X-7 | 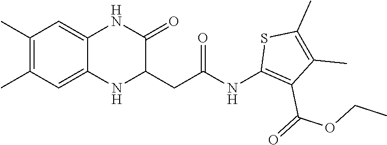 [SMILES: CCOC(=O)c1c(NC(=O)CC2Nc3cc(C)c(C)cc3NC2=O)sc(C)c1C] Leenus, M. et al., Cancer Res. 2014, 74(11), 3104-3113; hereby incorporated by reference. |
| X-8 | 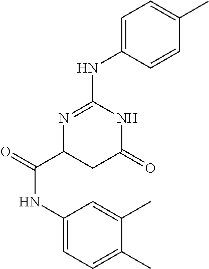 [SMILES: Cc1ccc(NC2=NC(C(=O)Nc3ccc(C)c(C)c3)CC(=O)N2)cc1] Leenus et al. 2014 |
| X-9 | 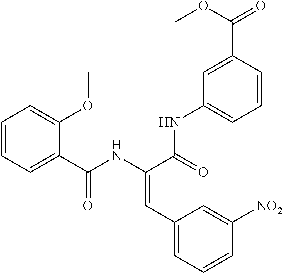 [SMILES: COC(=O)c1cccc(NC(=O)/C(=C\c2cccc([N+](=O)[O-])c2)NC(=O)c2ccccc2OC)c1] Leenus et al. 2014 |
| X-10 | 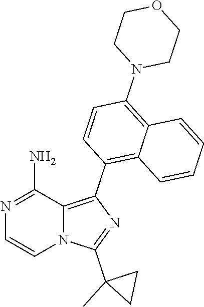 [SMILES: CC1(c2nc(-c3ccc(N4CCOCC4)c4ccccc34)c3c(N)nccn23)CC1] |
| X-11 | 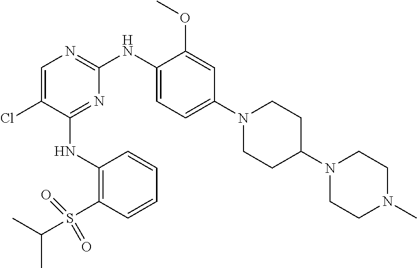 [SMILES: COc1cc(N2CCC(N3CCN(C)CC3)CC2)ccc1Nc1ncc(Cl)c(Nc2ccccc2S(=O)(=O)C(C)C)n1] |
| X-12 | 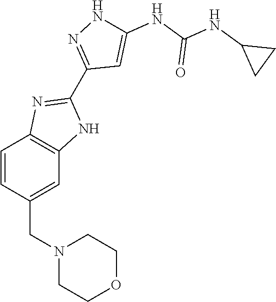 [SMILES: O=C(Nc1cc(-c2nc3ccc(CN4CCOCC4)cc3[nH]2)n[nH]1)NC1CC1] |
| X-13 | 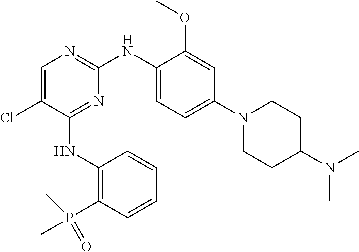 [SMILES: COc1cc(N2CCC(N(C)C)CC2)ccc1Nc1ncc(Cl)c(Nc2ccccc2P(C)(C)=O)n1] |
| X-14 | 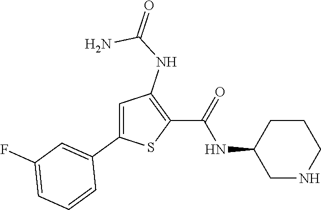 [SMILES: NC(=O)Nc1cc(-c2cccc(F)c2)sc1C(=O)N[C@H]1CCCNC1] |
| X-15 | 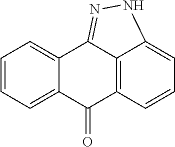 [SMILES: O=C1c2ccccc2-c2n[nH]c3cccc1c23] |
| X-15 | 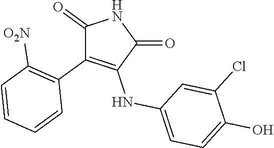 [SMILES: O=C1NC(=O)C(c2ccccc2[N+](=O)[O-])=C1Nc1ccc(O)c(Cl)c1] |
| X-16 | 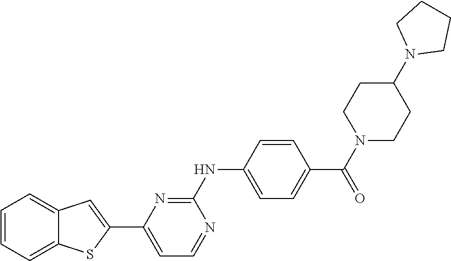 [SMILES: O=C(c1ccc(Nc2nccc(-c3cc4ccccc4s3)n2)cc1)N1CCC(N2CCCC2)CC1] |
| X-17 | 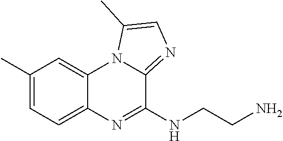 [SMILES: Cc1ccc2nc(NCCN)c3ncc(C)n3c2c1] IRE1 binders Feldman, H.C. et al., ACS Chem Bio 2016, 11, 2195-2205; hereby incorporated by reference. |
| X-18 | 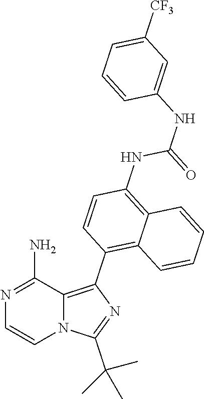 [SMILES: CC(C)(C)c1nc(-c2ccc(NC(=O)Nc3cccc(C(F)(F)F)c3)c3ccccc23)c2c(N)nccn12] Ghosh, R. et al., Cell 2014, 158, 534-548; hereby incorporated by reference. |
| X-19 | 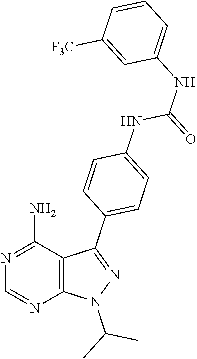 [SMILES: CC(C)n1nc(-c2ccc(NC(=O)Nc3cccc(C(F)(F)F)c3)cc2)c2c(N)ncnc21] |
| X-20 | 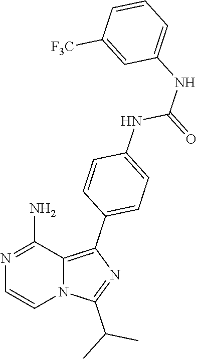 [SMILES: CC(C)c1nc(-c2ccc(NC(=O)Nc3cccc(C(F)(F)F)c3)cc2)c2c(N)nccn12] |
| X-21 | 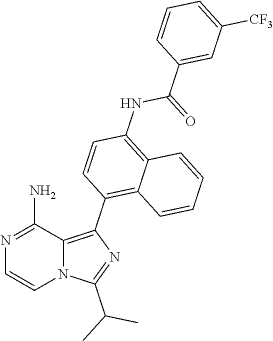 [SMILES: CC(C)c1nc(-c2ccc(NC(=O)c3cccc(C(F)(F)F)c3)c3ccccc23)c2c(N)nccn12] |
| X-22 | 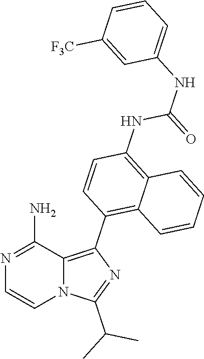 [SMILES: CC(C)c1nc(-c2ccc(NC(=O)Nc3cccc(C(F)(F)F)c3)c3ccccc23)c2c(N)nccn12] |
| X-23 | 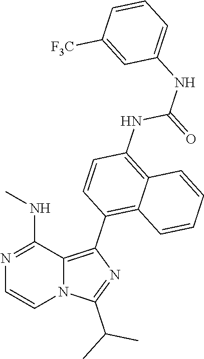 [SMILES: CNc1nccn2c(C(C)C)nc(-c3ccc(NC(=O)Nc4cccc(C(F)(F)F)c4)c4ccccc34)c12] Wang, L. et al., Nature Chem Bio 2012, 8, 982- 989; hereby incorporated by reference. |
| X-24 | 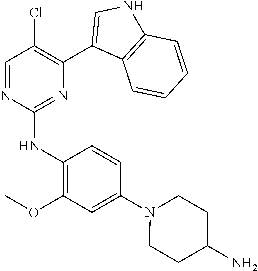 [SMILES: COc1cc(N2CCC(N)CC2)ccc1Nc1ncc(Cl)c(-c2c[nH]c3ccccc23)n1] |
| X-25 | 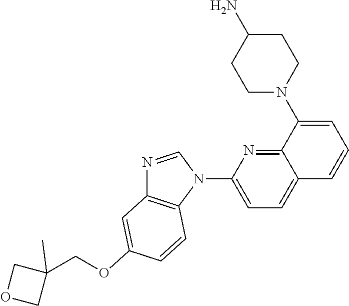 [SMILES: CC1(COc2ccc3c(c2)ncn3-c2ccc3cccc(N4CCC(N)CC4)c3n2)COC1] |
| X-26 | 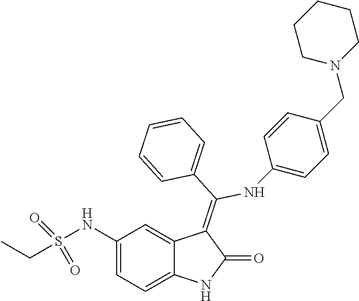 [SMILES: CCS(=O)(=O)Nc1ccc2c(c1)/C(=C(/Nc1ccc(CN3CCCCC3)cc1)c1ccccc1)C(=O)N2] |
| X-27 | 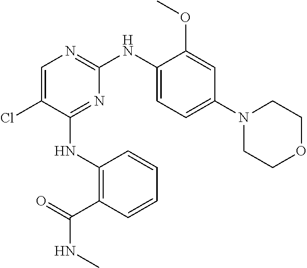 [SMILES: CNC(=O)c1ccccc1Nc1nc(Nc2ccc(N3CCOCC3)cc2OC)ncc1Cl] |
| X-28 | 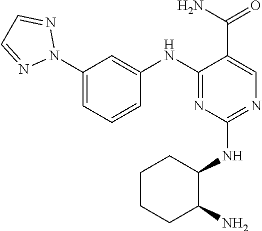 [SMILES: NC(=O)c1cnc(N[C@@H]2CCCC[C@@H]2N)nc1Nc1cccc(-n2nccn2)c1] |
| X-29 | 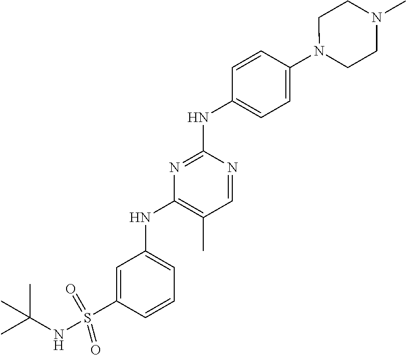 [SMILES: Cc1cnc(Nc2ccc(N3CCN(C)CC3)cc2)nc1Nc1cccc(S(=O)(=O)NC(C)(C)C)c1] |
| X-30 | 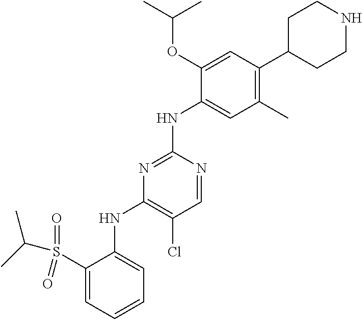 [SMILES: Cc1cc(Nc2ncc(Cl)c(Nc3ccccc3S(=O)(=O)C(C)C)n2)c(OC(C)C)cc1C1CCNCC1] |
| X-31 | 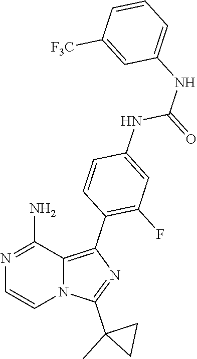 [SMILES: CC1(c2nc(-c3ccc(NC(=O)Nc4cccc(C(F)(F)F)c4)cc3F)c3c(N)nccn23)CC1] |
| X-32 | 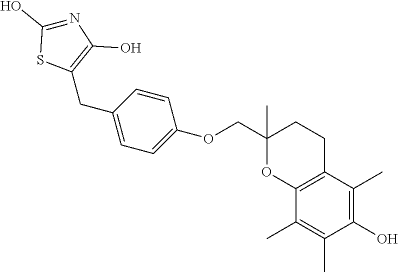 [SMILES: Cc1c(C)c2c(c(C)c1O)CCC(C)(COc1ccc(Cc3sc(O)nc3O)cc1)O2] |
| X-33 | 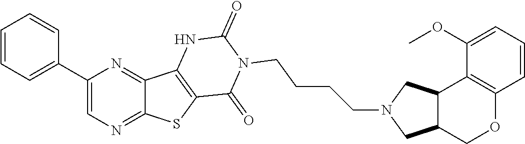 |
| X-34 | 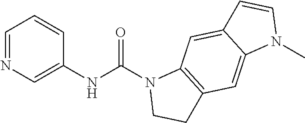 [SMILES: Cn1ccc2cc3c(cc21)CCN3C(=O)Nc1cccnc1] |
| X-35 | 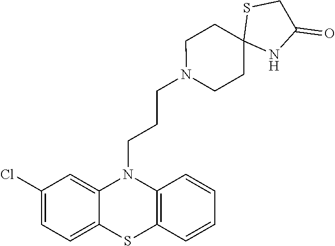 [SMILES: O=C1CSC2(CCN(CCCN3c4ccccc4Sc4ccc(Cl)cc43)CC2)N1] Poletto, M. et al., Mol Carcinog. 2016; 55(5): 688-704; hereby incorporated by reference. |
| X-36 | 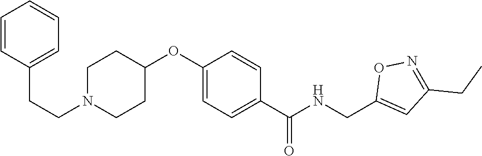 [SMILES: CCc1cc(CNC(=O)c2ccc(OC3CCN(CCc4ccccc4)CC3)cc2)on1] |
| X-37 | 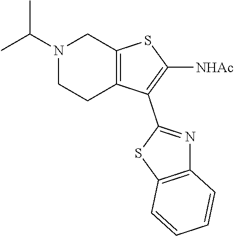 [SMILES: CC(=O)Nc1sc2c(c1-c1nc3ccccc3s1)CCN(C(C)C)C2] |
| X-38 | 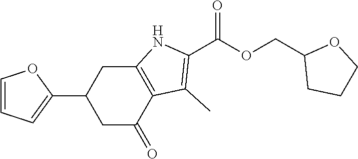 [SMILES: Cc1c(C(=O)OCC2CCCO2)[nH]c2c1C(=O)CC(c1ccco1)C2] |
| X-39 | 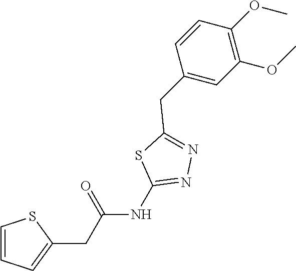 [SMILES: COc1ccc(Cc2nnc(NC(=O)Cc3cccs3)s2)cc1OC] |
| X-40 | 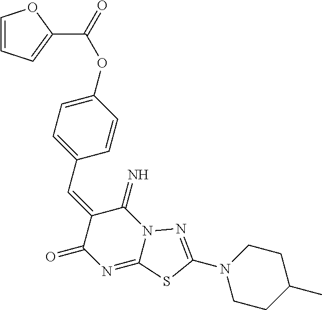 [SMILES: CC1CCN(C2=NN3C(=N)/C(=C\c4ccc(OC(=O)c5ccco5)cc4)C(=O)N=C3S2)CC1] |
| X-41 | 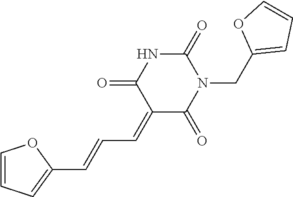 [SMILES: O=C1NC(=O)N(Cc2ccco2)C(=O)/C1=C/C=C/c1ccco1] |
| X-42 | 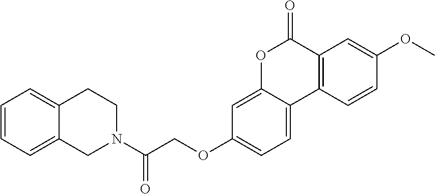 [SMILES: COc1ccc2c(c1)c(=O)oc1cc(OCC(=O)N3CCc4ccccc4C3)ccc12] |
| X-43 | 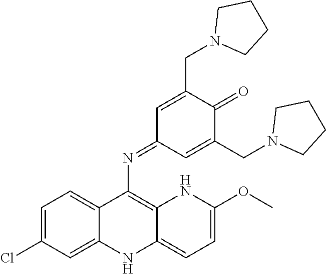 [SMILES: COC1=CC=C2Nc3cc(Cl)ccc3C(N=C3C=C(CN4CCCC4)C(=O)C(CN4CCCC4)=C3)=C2N1] |
| X-44 | 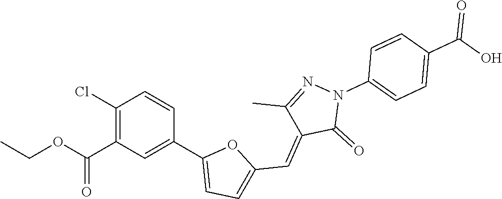 [SMILES: CCOC(=O)c1cc(-c2ccc(/C=C3/C(=O)N(c4ccc(C(=O)O)cc4)N=C3C)o2)ccc1Cl] |
| X-45 | 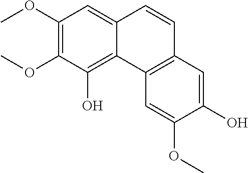 [SMILES: COc1cc2c(ccc3cc(OC)c(OC)c(O)c32)cc1O] |
| X-46 | 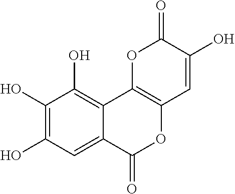 [SMILES: O=c1oc2c(cc1O)oc(=O)c1cc(O)c(O)c(O)c12] Dorjsuren, D. et al., PLOS One 2012, 7, e47974; hereby incorporated by reference. |
| X-47 | 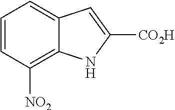 [SMILES: O=C(O)c1cc2cccc([N+](=O)[O-])c2[nH]1] Madhusudan, S., et al. 2005. Nucl. Acids Res.33, 4711; hereby incorporated by reference. |
| X-48 | 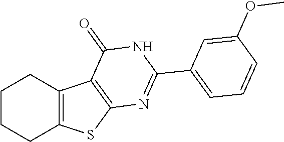 [SMILES: COc1cccc(-c2nc3sc4c(c3c(=O)[nH]2)CCCC4)c1] |
| X-49 | 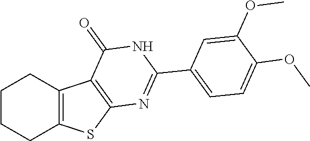 [SMILES: COc1ccc(-c2nc3sc4c(c3c(=O)[nH]2)CCCC4)cc1OC] |
| X-50 | 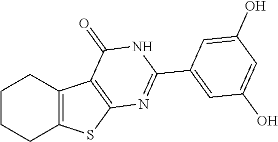 [SMILES: O=c1[nH]c(-c2cc(O)cc(O)c2)nc2sc3c(c12)CCCC3] Ligand for, e.g., RNAse L |
| X-51 | 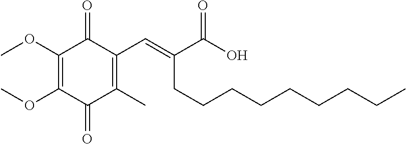 Ligand for, e.g., APE1 |
| X-52 | 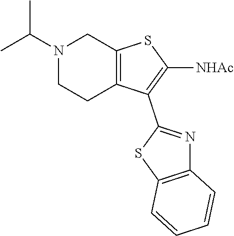 Ligand for, e.g., APE1 |
| X-53 | 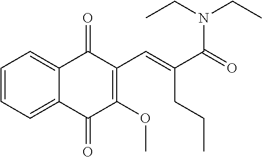 Ligand for, e.g., APE1 |
| X-54 | 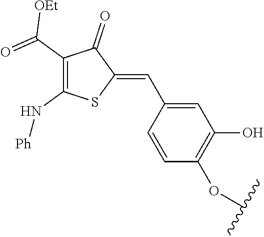 [SMILES: *Oc1ccc(/C=C2\SC(Nc3ccccc3)=C(C(=O)OCC)C2=O)cc1O] Ligand for, e.g., RNAseL; wavy bond indicates point of attachment to L in Formula A |

### Linkers

As defined generally above, the linker, L, in the formulae described herein is a bivalent group that connects the rSM to the ligand for the decay factor ligand (DFL). In some embodiments, L is a covalent bond or a bivalent, saturated or unsaturated, straight or branched, optionally substituted C₁₋₅₀ hydrocarbon chain, wherein 0, 1, 2, 3, 4, 5, 6, 7, 8, 9, or 10 methylene units of L are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(NR₂)—, an amino acid, wherein:

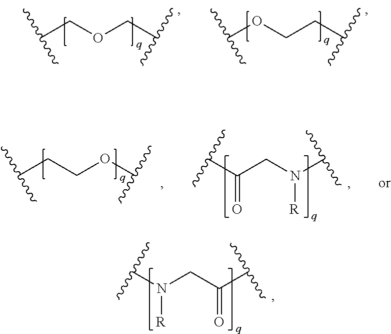

each -Cy²- is independently an optionally substituted bivalent ring selected from phenylenyl, an 8-12 membered bicyclic arylenyl, a 3-8 membered saturated or partially unsaturated carbocyclylenyl, an 8-12 membered bicyclic saturated or partially unsaturated carbocyclylenyl, a 3-8 membered saturated or partially unsaturated heterocyclylenyl having 1-3 heteroatoms independently selected from nitrogen, oxygen, and sulfur, an 8-12 membered bicyclic saturated or partially unsaturated heterocyclylenyl having 1-5 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
wherein each q is independently 1, 2, or 3.

In some embodiments, L is a covalent bond. In some embodiments, L is a bivalent, saturated or unsaturated, straight or branched, optionally substituted C₁₋₅₀ hydrocarbon chain, wherein 0, 1, 2, 3, 4, 5, 6, 7, 8, 9, or 10 methylene units of L are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(NR₂)—, an amino acid,

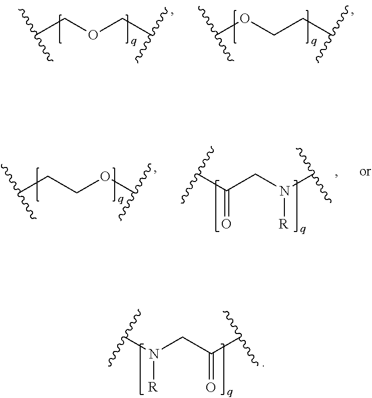

In some embodiments, L is a bivalent, saturated or unsaturated, straight or branched, optionally substituted C₁₋₅₀, C₁₋₄₀, C₁₋₃₀, C₁₋₂₀, C₁₋₁₅, C₁₋₁₀, C₁₋₅, C₂₋₅₀, C₂₋₄₀, C₂₋₃₀, C₂₋₂₀, C₂₋₁₅, C₂₋₁₀, C₃₋₅₀, C₃₋₄₀, C₃₋₃₀, C₃₋₂₀, C₃₋₁₅, C₃₋₁₀, C₄₋₅₀, C₄₋₄₀, C₄₋₃₀, C₄₋₂₀, C₄₋₁₅, C₄₋₁₀, C₅₋₅₀, C₅₋₄₀, C₅₋₃₀, C₅₋₂₀, C₅₋₁₅, C₅₋₁₀, C₆₋₅₀, C₆₋₄₀, C₆₋₃₀, C₆₋₂₀, C₆₋₁₅, C₇₋₅₀, C₇₋₄₀, C₇₋₃₀, C₇₋₂₀, C₇₋₁₅, C₈₋₅₀, C₈₋₄₀, C₈₋₃₀, C₈₋₂₀, C₈₋₁₅, C₁₀₋₅₀, C₁₀₋₄₀, C₁₀₋₃₀, C₁₀₋₂₀, C₁₀₋₁₅, C₁₂₋₅₀, C₁₂₋₄₀, C₁₂₋₃₀, C₁₂₋₂₀, C₁₅₋₅₀, C₁₅₋₄₀, C₁₅₋₃₀, C₁₅₋₂₀, C₂₀₋₅₀, C₂₀₋₄₀, or C₂₀₋₃₀ hydrocarbon chain, wherein 0, 1, 2, 3, 4, 5, 6, 7, 8, 9, or 10 methylene units of L are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(NR₂)—, an amino acid,

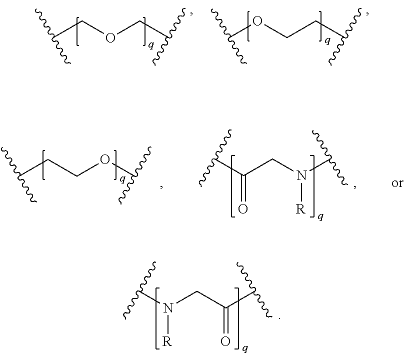

In some embodiments, L is a bivalent, saturated or unsaturated, straight or branched, optionally substituted C₁₋₅₀, C₁₋₄₀, C₁₋₃₀, C₁₋₂₀, C₁₋₁₅, C₁₋₁₀, C₁₋₅, C₂₋₅₀, C₂₋₄₀, C₂₋₃₀, C₂₋₂₀, C₂₋₁₅, C₂₋₁₀, C₃₋₅₀, C₃₋₄₀, C₃₋₃₀, C₃₋₂₀, C₃₋₁₅, C₃₋₁₀, C₄₋₅₀, C₄₋₄₀, C₄₋₃₀, C₄₋₂₀, C₄₋₁₅, C₄₋₁₀, C₅₋₅₀, C₅₋₄₀, C₅₋₃₀, C₅₋₂₀, C₅₋₁₅, C₅₋₁₀, C₆₋₅₀, C₆₋₄₀, C₆₋₃₀, C₆₋₂₀, C₆₋₁₅, C₇₋₅₀, C₇₋₄₀, C₇₋₃₀, C₇₋₂₀, C₇₋₁₅, C₈₋₅₀, C₈₋₄₀, C₈₋₃₀, C₈₋₂₀, C₈₋₁₅, C₁₀₋₅₀, C₁₀₋₄₀, C₁₀₋₃₀, C₁₀₋₂₀, C₁₀₋₁₅, C₁₂₋₅₀, C₁₂₋₄₀, C₁₂₋₃₀, C₁₂₋₂₀, C₁₅₋₅₀, C₁₅₋₄₀, C₁₅₋₃₀, C₁₅₋₂₀, C₂₀₋₅₀, C₂₀₋₄₀, or C₂₀₋₃₀ hydrocarbon chain, wherein 0, 1, 2, 3, 4, 5, 6, 7, 8, 9, or 10 methylene units of L are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(NR₂)—, an amino acid,

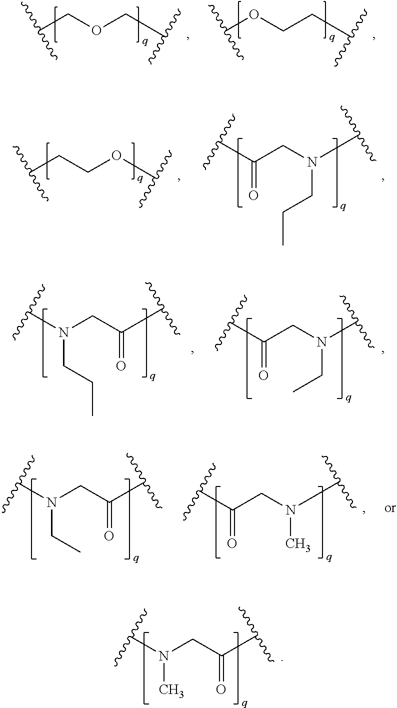

In some embodiments, L is a bivalent, saturated or unsaturated, straight or branched, optionally substituted C₁₋₅₀, C₁₋₄₀, C₁₋₃₀, C₁₋₂₀, C₁₋₁₅, C₁₋₁₀, C₁₋₅, C₂₋₅₀, C₂₋₄₀, C₂₋₃₀, C₂₋₂₀, C₂₋₁₅, C₂₋₁₀, C₃₋₅₀, C₃₋₄₀, C₃₋₃₀, C₃₋₂₀, C₃₋₁₅, C₃₋₁₀, C₄₋₅₀, C₄₋₄₀, C₄₋₃₀, C₄₋₂₀, C₄₋₁₅, C₄₋₁₀, C₅₋₅₀, C₅₋₄₀, C₅₋₃₀, C₅₋₂₀, C₅₋₁₅, C₅₋₁₀, C₆₋₅₀, C₆₋₄₀, C₆₋₃₀, C₆₋₂₀, C₆₋₁₅, C₇₋₅₀, C₇₋₄₀, C₇₋₃₀, C₇₋₂₀, C₇₋₁₅, C₈₋₅₀, C₈₋₄₀, C₈₋₃₀, C₈₋₂₀, C₈₋₁₅, C₁₀₋₅₀, C₁₀₋₄₀, C₁₀₋₃₀, C₁₀₋₂₀, C₁₀₋₁₅, C₁₂₋₅₀, C₁₂₋₄₀, C₁₂₋₃₀, C₁₂₋₂₀, C₁₅₋₅₀, C₁₅₋₄₀, C₁₅₋₃₀, C₁₅₋₂₀, C₂₀₋₅₀, C₂₀₋₄₀, or C₂₀₋₃₀ hydrocarbon chain, wherein 0, 1, 2, 3, 4, 5, 6, 7, or 8 methylene units of L are independently replaced by -Cy²-, —O—, —N(R)—, —S—, —OC(O)—, —C(O)O—, —C(O)—, —C(S)—, —S(O)—, —S(O)₂—, —N(R)C(O)—, —C(O)N(R)—, —OC(O)N(R)—, —N(R)C(O)O—, an amino acid,

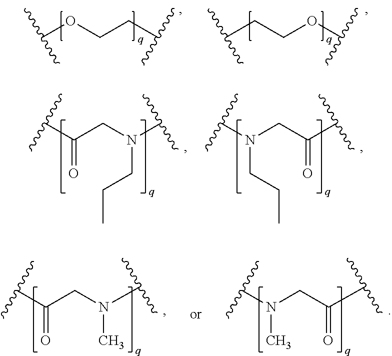

In some embodiments, L comprises 1, 2, 3, 4, 5, or 6 PEG units,

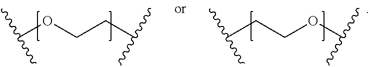

In some embodiments, L comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 PEG units. In some embodiments, L comprises 1, 2, 3, 4, 5, 6, 7, 8, 9, 10, 11, or 12 units; or 1, 2, 3, 4, 5, or 6 units, of

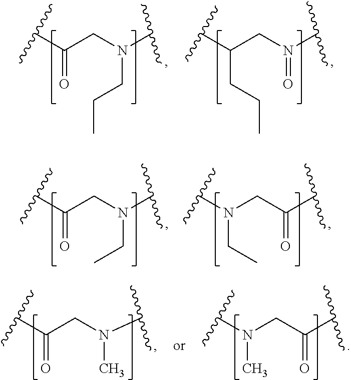

In some embodiments, L is a saturated chain. In some embodiments, L comprises at least one unsaturated pair of carbon atoms, i.e., at least one double or triple carbon-carbon bond. In some embodiments, L comprises 1, 2, 3, 4, or 5 double or triple carbon-carbon bonds. In some embodiments, L is a straight hydrocarbon chain wherein methylene units of L are optionally replaced or substituted as described above. In some embodiments, L is a saturated, straight hydrocarbon chain wherein methylene units of L are optionally replaced or substituted as described above.

In some embodiments, L is substituted with 1, 2, 3, 4, 5, 6, 7, 8, 9, or 10 “optional substituents” as defined herein. In some embodiments, each substituent is independently selected from deuterium, halogen, —CN, —OR, —NR₂, —SR, a 3-8 membered saturated or partially unsaturated monocyclic carbocyclic ring, phenyl optionally substituted with one or more C₁₋₄ alkyl, —CO₂R, —OR, —CONR₂, —NR₂, or halogen, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, an 8-10 membered bicyclic heteroaromatic ring having 1-5 heteroatoms independently selected from nitrogen, oxygen, or sulfur, or a C₁₋₆ aliphatic group optionally substituted with —CN, —OR, —NR₂, —SR, a 3-8 membered saturated or partially unsaturated monocyclic carbocyclic ring, phenyl optionally substituted with one or more C₁₋₄ alkyl, —CO₂R, —OR, —CONR₂, —NR₂, or halogen, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur, or the C₁₋₆ aliphatic is optionally substituted with 1, 2, 3, 4, 5, or 6 deuterium or halogen atoms; or two substituents attached to the same carbon atom, taken together with the carbon atom to which they are attached, form a 3-6 membered saturated monocyclic carbocyclic ring or 3-6 membered saturated heterocyclic ring having 1-2 heteroatoms independently selected from nitrogen, oxygen, or sulfur.

As described above, in some embodiments, a methylene unit of L is replaced with an amino acid. The amino acid may be naturally-occurring or non-naturally occurring. In some embodiments, the amino acid is selected from a non-polar or branched chain amino acid (BCAA). In some embodiments, the amino acid is selected from valine, isoleucine, leucine, methionine, alanine, proline, glycine, phenylalanine, tyrosine, tryptophan, histidine, asparagine, glutamine, serine threonine, lysine, arginine, histidine, aspartic acid, glutamic acid, cysteine, selenocysteine, or tyrosine. In some embodiments, the amino acid is an L-amino acid. In some embodiments, the amino acid is a D-amino acid.

In some embodiments, L is

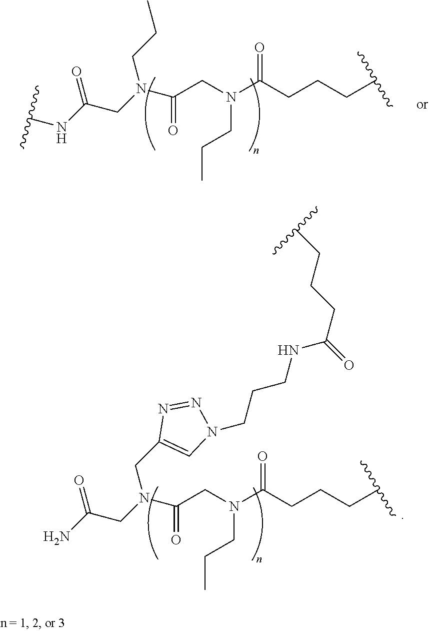

In some embodiments, L is selected from one of those depicted in Table 2, below.

TABLE 2
Exemplary Linkers

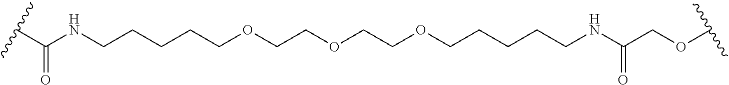 [SMILES: *OCC(=O)NCCCCCOCCOCCOCCCCCNC(*)=O]

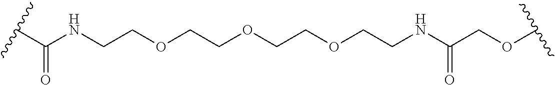 [SMILES: *OCC(=O)NCCOCCOCCOCCNC(*)=O]

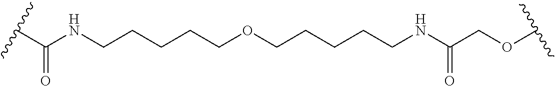 [SMILES: *OCC(=O)NCCCCCOCCCCCNC(*)=O]

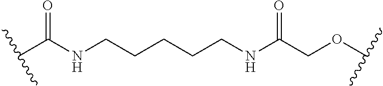 [SMILES: *OCC(=O)NCCCCCNC(*)=O]

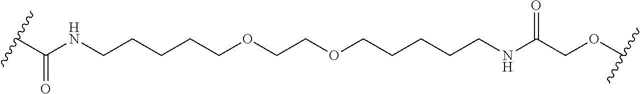 [SMILES: *OCC(=O)NCCCCCOCCOCCCCCNC(*)=O]

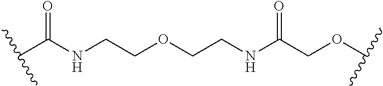 [SMILES: *OCC(=O)NCCOCCNC(*)=O]

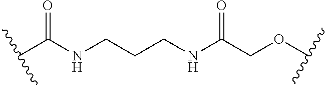 [SMILES: *OCC(=O)NCCCNC(*)=O]

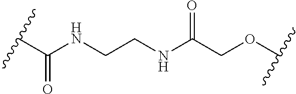 [SMILES: *OCC(=O)NCCNC(*)=O]

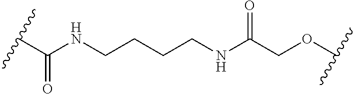 [SMILES: *OCC(=O)NCCCCNC(*)=O]

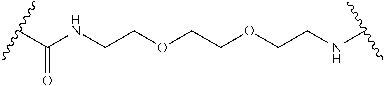 [SMILES: *NCCOCCOCCNC(*)=O]

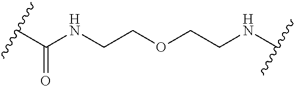 [SMILES: *NCCOCCNC(*)=O]

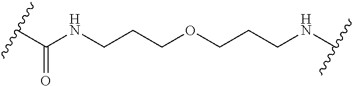 [SMILES: *NCCCOCCCNC(*)=O]

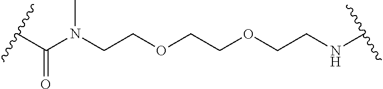 [SMILES: *NCCOCCOCCN(C)C(*)=O]

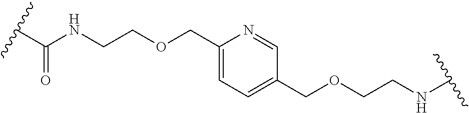 [SMILES: *NCCOCc1ccc(COCCNC(*)=O)nc1]

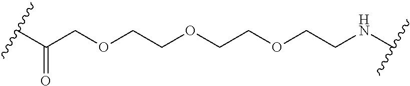 [SMILES: *NCCOCCOCCOCC(*)=O]

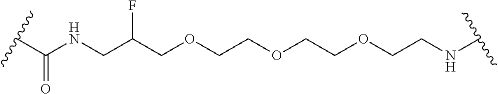 [SMILES: *NCCOCCOCCOCC(F)CNC(*)=O]

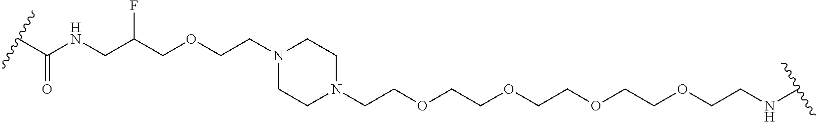 [SMILES: *NCCOCCOCCOCCOCCN1CCN(CCOCC(F)CNC(*)=O)CC1]

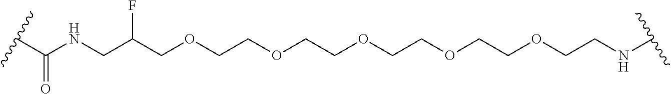 [SMILES: *NCCOCCOCCOCCOCCOCC(F)CNC(*)=O]

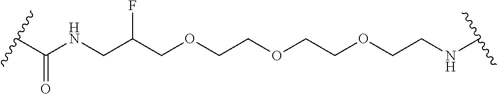 [SMILES: *NCCOCCOCCOCC(F)CNC(*)=O]

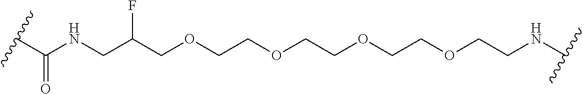 [SMILES: *NCCOCCOCCOCCOCC(F)CNC(*)=O]

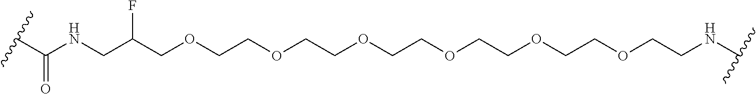 [SMILES: *NCCOCCOCCOCCOCCOCCOCC(F)CNC(*)=O]

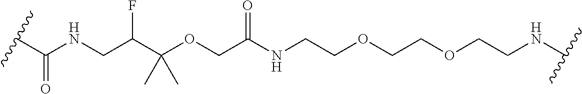 [SMILES: *NCCOCCOCCNC(=O)COC(C)(C)C(F)CNC(*)=O]

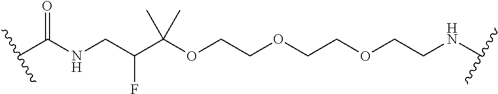 [SMILES: *NCCOCCOCCOC(C)(C)C(F)CNC(*)=O]

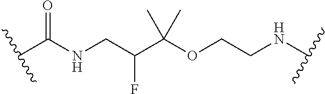 [SMILES: *NCCOC(C)(C)C(F)CNC(*)=O]

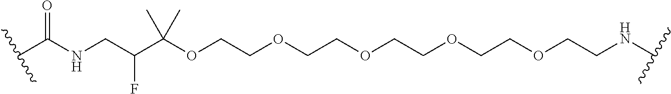 [SMILES: *NCCOCCOCCOCCOCCOC(C)(C)C(F)CNC(*)=O]

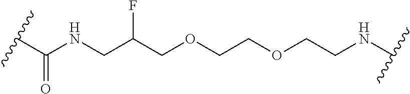 [SMILES: *NCCOCCOCC(F)CNC(*)=O]

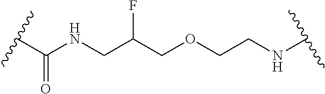 [SMILES: *NCCOCC(F)CNC(*)=O]

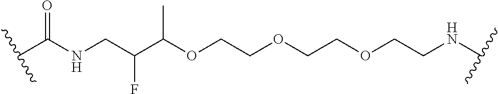 [SMILES: *NCCOCCOCCOC(C)C(F)CNC(*)=O]

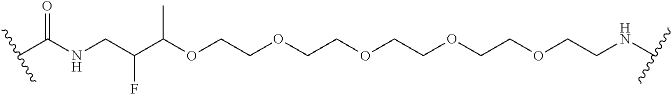 [SMILES: *NCCOCCOCCOCCOCCOC(C)C(F)CNC(*)=O]

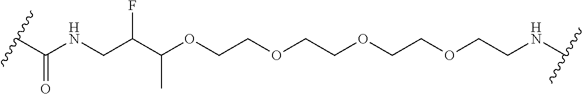 [SMILES: *NCCOCCOCCOCCOC(C)C(F)CNC(*)=O]

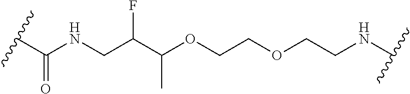 [SMILES: *NCCOCCOC(C)C(F)CNC(*)=O]

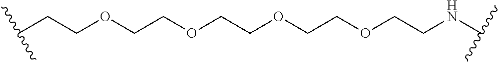 [SMILES: *CCOCCOCCOCCOCCN*]

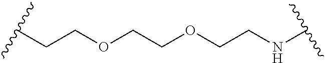 [SMILES: *CCOCCOCCN*]

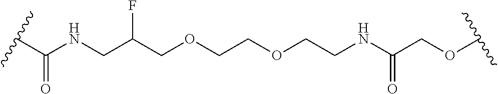 [SMILES: *OCC(=O)NCCOCCOCC(F)CNC(*)=O]

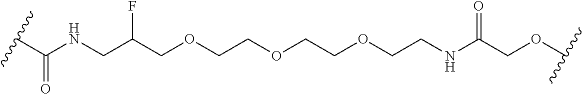 [SMILES: *OCC(=O)NCCOCCOCCOCC(F)CNC(*)=O]

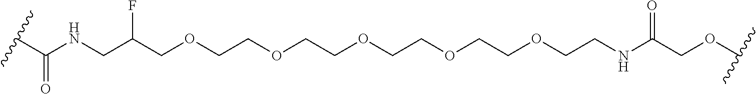 [SMILES: *OCC(=O)NCCOCCOCCOCCOCCOCC(F)CNC(*)=O]

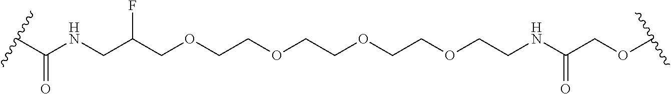 [SMILES: *OCC(=O)NCCOCCOCCOCCOCC(F)CNC(*)=O]

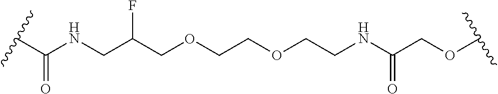 [SMILES: *OCC(=O)NCCOCCOCC(F)CNC(*)=O]

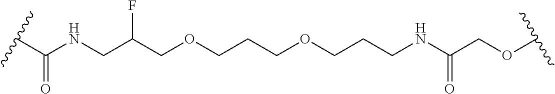 [SMILES: *OCC(=O)NCCCOCCCOCC(F)CNC(*)=O]

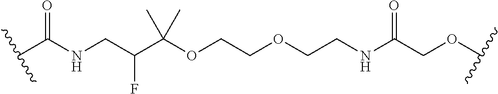 [SMILES: *OCC(=O)NCCOCCOC(C)(C)C(F)CNC(*)=O]

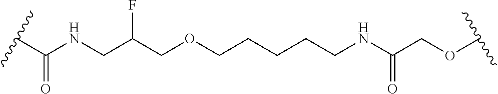 [SMILES: *OCC(=O)NCCCCCOCC(F)CNC(*)=O]

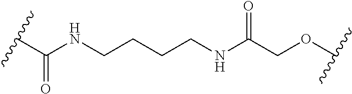 [SMILES: *OCC(=O)NCCCCNC(*)=O]

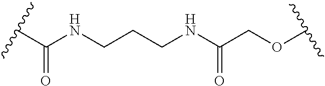 [SMILES: *OCC(=O)NCCCNC(*)=O]

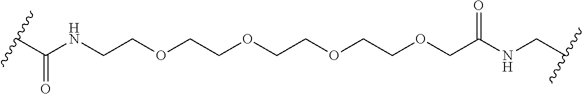 [SMILES: *CNC(=O)COCCOCCOCCOCCNC(*)=O]

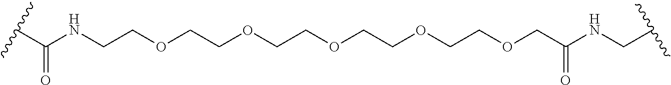 [SMILES: *CNC(=O)COCCOCCOCCOCCOCCNC(*)=O]

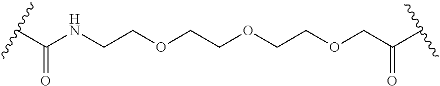 [SMILES: *C(=O)COCCOCCOCCNC(*)=O]

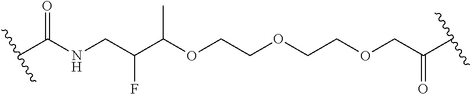 [SMILES: *C(=O)COCCOCCOC(C)C(F)CNC(*)=O]

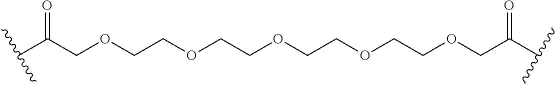 [SMILES: *C(=O)COCCOCCOCCOCCOCC(*)=O]

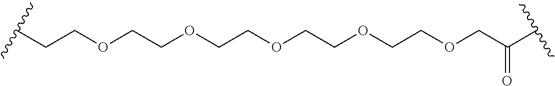 [SMILES: *CCOCCOCCOCCOCCOCC(*)=O]

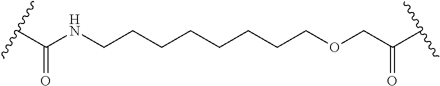 [SMILES: *C(=O)COCCCCCCCCNC(*)=O]

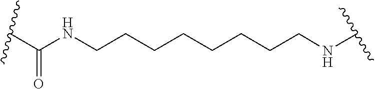 [SMILES: *NCCCCCCCCNC(*)=O]

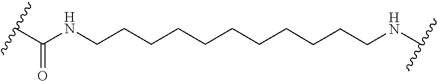 [SMILES: *NCCCCCCCCCCCNC(*)=O]

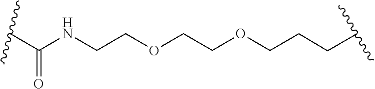 [SMILES: *CCCOCCOCCNC(*)=O]

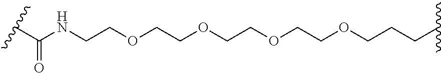 [SMILES: *CCCOCCOCCOCCOCCNC(*)=O]

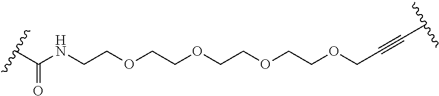 [SMILES: *C#CCOCCOCCOCCOCCNC(*)=O]

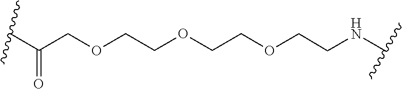 [SMILES: *NCCOCCOCCOCC(*)=O]

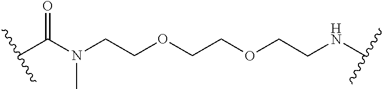 [SMILES: *NCCOCCOCCN(C)C(*)=O]

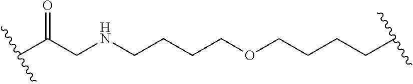 [SMILES: *CCCCOCCCCNCC(*)=O]

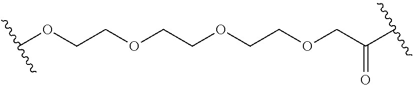 [SMILES: *OCCOCCOCCOCC(*)=O]

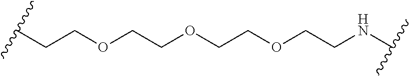 [SMILES: *CCOCCOCCOCCN*]

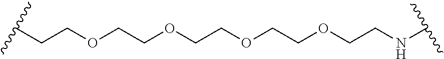 [SMILES: *CCOCCOCCOCCOCCN*]

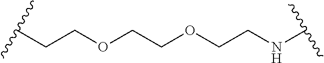 [SMILES: *CCOCCOCCN*]

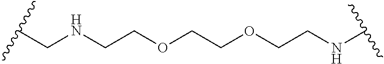 [SMILES: *CNCCOCCOCCN*]

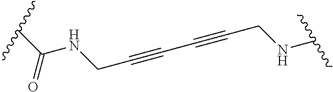 [SMILES: *NCC#CC#CCNC(*)=O]

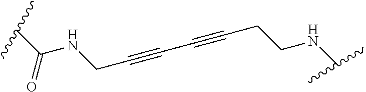 [SMILES: *NCCC#CC#CCNC(*)=O]

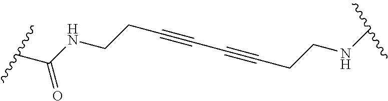 [SMILES: *NCCC#CC#CCCNC(*)=O]

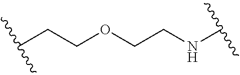 [SMILES: *CCOCCN*]

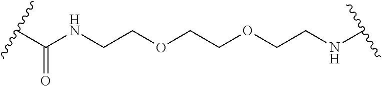 [SMILES: *NCCOCCOCCNC(*)=O]

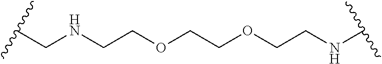 [SMILES: *CNCCOCCOCCN*]

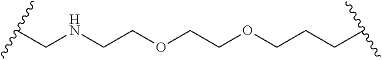 [SMILES: *CCCOCCOCCNC*]

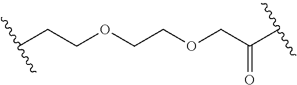 [SMILES: *CCOCCOCC(*)=O]

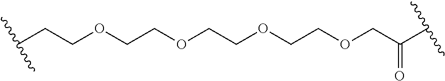 [SMILES: *CCOCCOCCOCCOCC(*)=O]

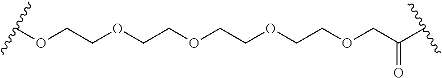 [SMILES: *OCCOCCOCCOCCOCC(*)=O]

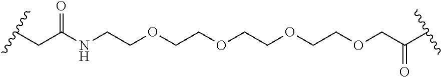 [SMILES: *CC(=O)NCCOCCOCCOCCOCC(*)=O]

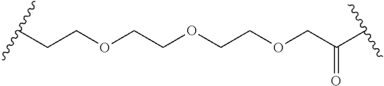 [SMILES: *CCOCCOCCOCC(*)=O]

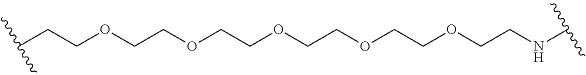 [SMILES: *CCOCCOCCOCCOCCOCCN*]

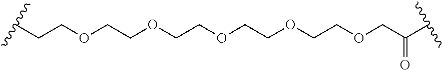 [SMILES: *CCOCCOCCOCCOCCOCC(*)=O]

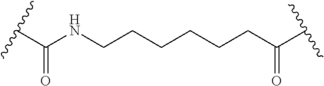 [SMILES: *C(=O)CCCCCCNC(*)=O]

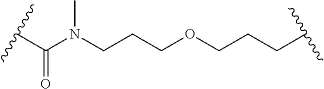 [SMILES: *CCCOCCCN(C)C(*)=O]

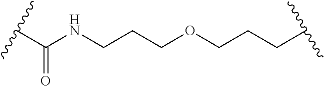 [SMILES: *CCCOCCCNC(*)=O]

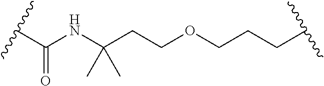 [SMILES: *CCCOCCC(C)(C)NC(*)=O]

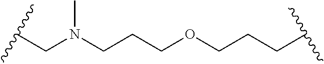 [SMILES: *CCCOCCCN(C)C*]

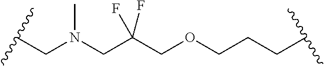 [SMILES: *CCCOCC(F)(F)CN(C)C*]

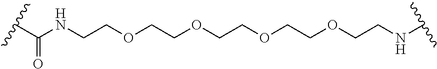 [SMILES: *NCCOCCOCCOCCOCCNC(*)=O]

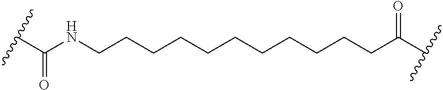 [SMILES: *C(=O)CCCCCCCCCCCNC(*)=O]

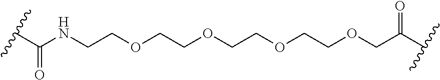 [SMILES: *C(=O)COCCOCCOCCOCCNC(*)=O]

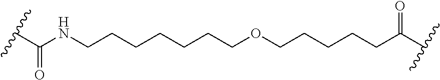 [SMILES: *C(=O)CCCCCOCCCCCCCNC(*)=O]

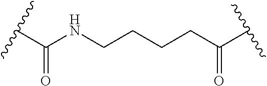 [SMILES: *C(=O)CCCCNC(*)=O]

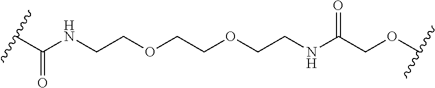 [SMILES: *OCC(=O)NCCOCCOCCNC(*)=O]

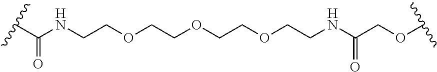 [SMILES: *OCC(=O)NCCOCCOCCOCCNC(*)=O]

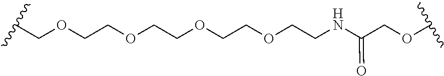 [SMILES: *COCCOCCOCCOCCNC(=O)CO*]

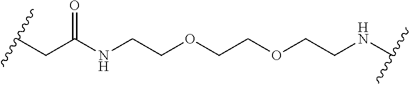 [SMILES: *CC(=O)NCCOCCOCCN*]

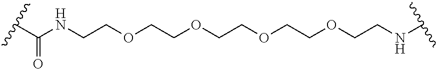 [SMILES: *NCCOCCOCCOCCOCCNC(*)=O]

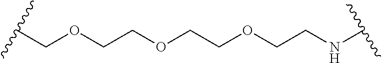 [SMILES: *COCCOCCOCCN*]

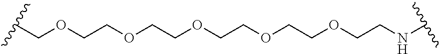 [SMILES: *COCCOCCOCCOCCOCCN*]

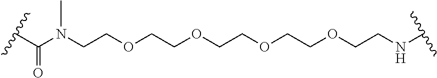 [SMILES: *NCCOCCOCCOCCOCCN(C)C(*)=O]

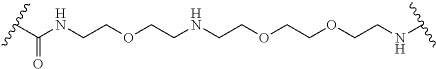 [SMILES: *NCCOCCOCCNCCOCCNC(*)=O]

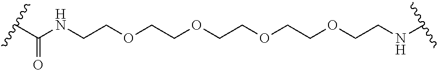 [SMILES: *NCCOCCOCCOCCOCCNC(*)=O]

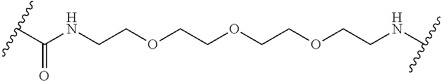 [SMILES: *NCCOCCOCCOCCNC(*)=O]

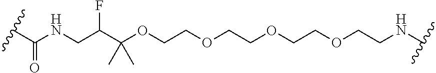 [SMILES: *NCCOCCOCCOCCOC(C)(C)C(F)CNC(*)=O]

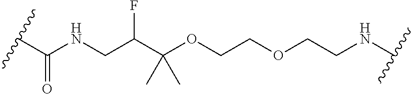 [SMILES: *NCCOCCOC(C)(C)C(F)CNC(*)=O]

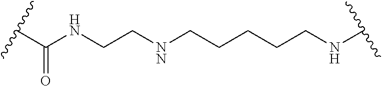

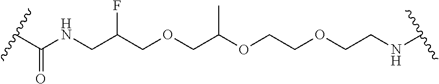 [SMILES: *NCCOCCOC(C)COCC(F)CNC(*)=O]

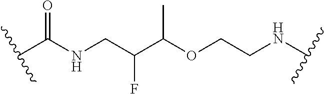 [SMILES: *NCCOC(C)C(F)CNC(*)=O]

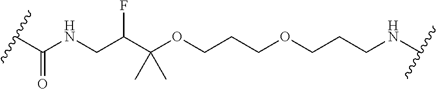 [SMILES: *NCCCOCCCOC(C)(C)C(F)CNC(*)=O]

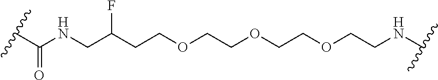 [SMILES: *NCCOCCOCCOCCC(F)CNC(*)=O]

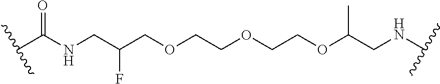 [SMILES: *NCC(C)OCCOCCOCC(F)CNC(*)=O]

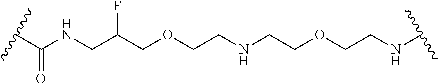 [SMILES: *NCCOCCNCCOCC(F)CNC(*)=O]

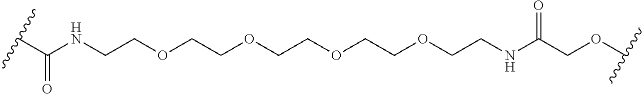 [SMILES: *OCC(=O)NCCOCCOCCOCCOCCNC(*)=O]

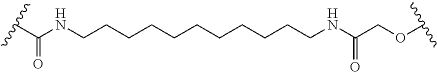 [SMILES: *OCC(=O)NCCCCCCCCCCCNC(*)=O]

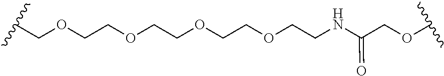 [SMILES: *COCCOCCOCCOCCNC(=O)CO*]

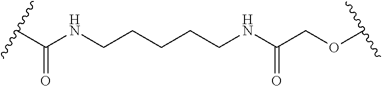 [SMILES: *OCC(=O)NCCCCCNC(*)=O]

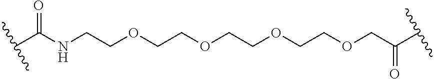 [SMILES: *C(=O)COCCOCCOCCOCCNC(*)=O]

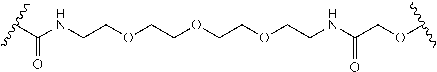 [SMILES: *OCC(=O)NCCOCCOCCOCCNC(*)=O]

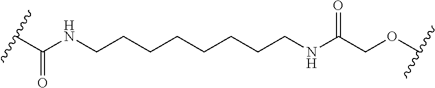 [SMILES: *OCC(=O)NCCCCCCCCNC(*)=O]

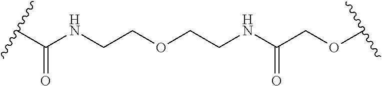 [SMILES: *OCC(=O)NCCOCCNC(*)=O]

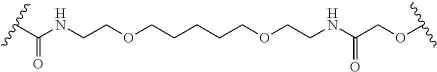 [SMILES: *OCC(=O)NCCOCCCCCOCCNC(*)=O]

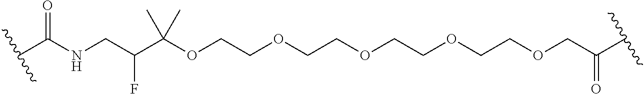 [SMILES: *C(=O)COCCOCCOCCOCCOC(C)(C)C(F)CNC(*)=O]

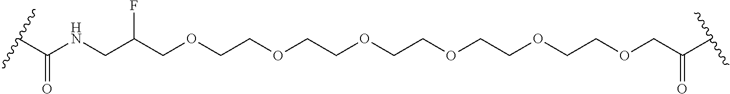 [SMILES: *C(=O)COCCOCCOCCOCCOCCOCC(F)CNC(*)=O]

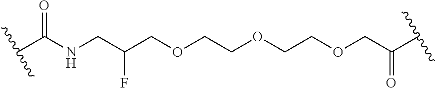 [SMILES: *C(=O)COCCOCCOCC(F)CNC(*)=O]

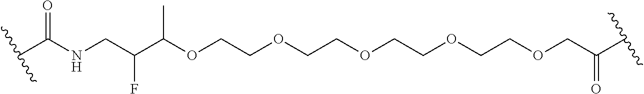 [SMILES: *C(=O)COCCOCCOCCOCCOC(C)C(F)CNC(*)=O]

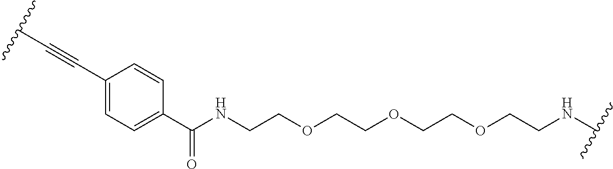 [SMILES: *C#Cc1ccc(C(=O)NCCOCCOCCOCCN*)cc1]

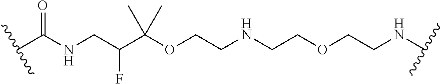 [SMILES: *NCCOCCNCCOC(C)(C)C(F)CNC(*)=O]

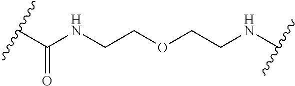 [SMILES: *NCCOCCNC(*)=O]

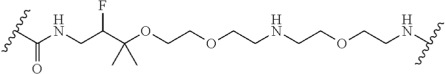 [SMILES: *NCCOCCNCCOCCOC(C)(C)C(F)CNC(*)=O]

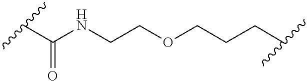 [SMILES: *CCCOCCNC(*)=O]

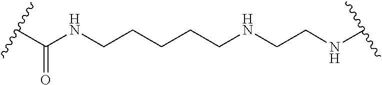 [SMILES: *NCCNCCCCCNC(*)=O]

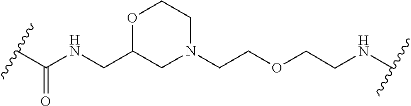 [SMILES: *NCCOCCN1CCOC(CNC(*)=O)C1]

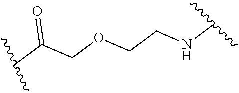 [SMILES: *NCCOCC(*)=O]

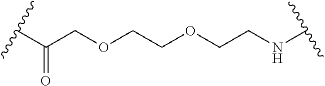 [SMILES: *NCCOCCOCC(*)=O]

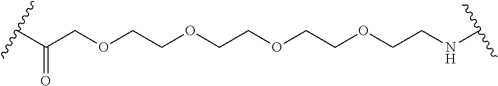 [SMILES: *NCCOCCOCCOCCOCC(*)=O]

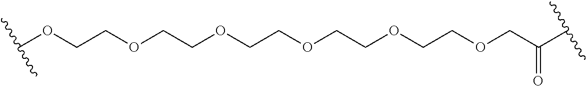 [SMILES: *OCCOCCOCCOCCOCCOCC(*)=O]

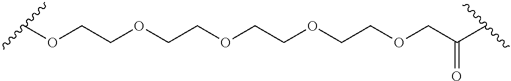 [SMILES: *OCCOCCOCCOCCOCC(*)=O]

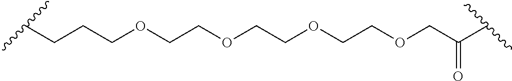 [SMILES: *CCCOCCOCCOCCOCC(*)=O]

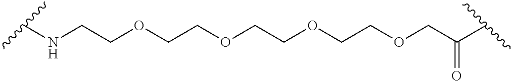 [SMILES: *NCCOCCOCCOCCOCC(*)=O]

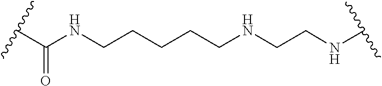 [SMILES: *NCCNCCCCCNC(*)=O]

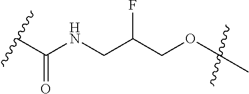 [SMILES: *OCC(F)CNC(*)=O]

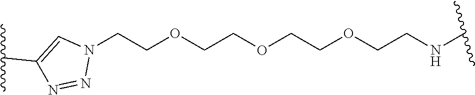 [SMILES: *NCCOCCOCCOCCn1cc(*)nn1]

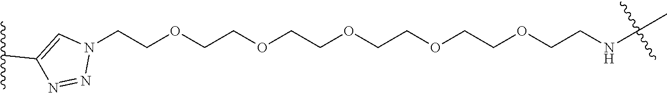 [SMILES: *NCCOCCOCCOCCOCCOCCn1cc(*)nn1]

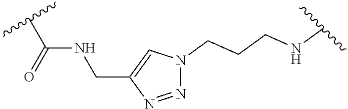 [SMILES: *NCCCn1cc(CNC(*)=O)nn1]

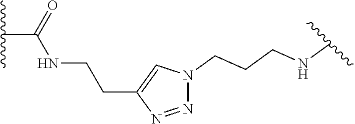 [SMILES: *NCCCn1cc(CCNC(*)=O)nn1]

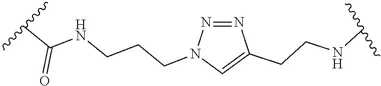 [SMILES: *NCCc1cn(CCCNC(*)=O)nn1]

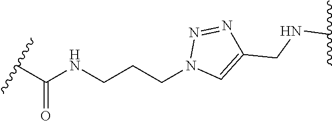 [SMILES: *NCc1cn(CCCNC(*)=O)nn1]

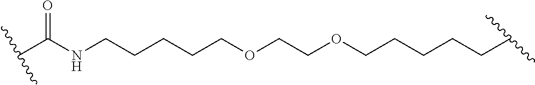 [SMILES: *CCCCCOCCOCCCCCNC(*)=O]

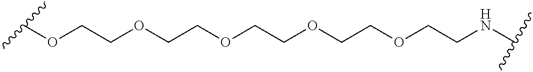 [SMILES: *NCCOCCOCCOCCOCCO*]

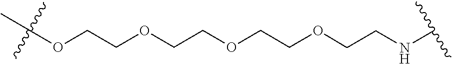 [SMILES: *NCCOCCOCCOCCO*]

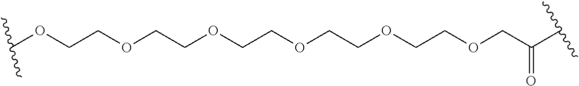 [SMILES: *OCCOCCOCCOCCOCCOCC(*)=O]

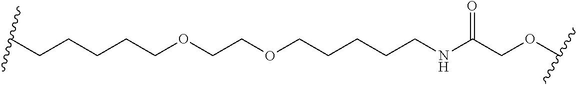 [SMILES: *CCCCCOCCOCCCCCNC(=O)CO*]

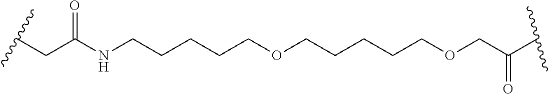 [SMILES: *CC(=O)NCCCCCOCCCCCOCC(*)=O]

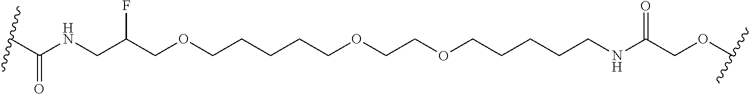 [SMILES: *OCC(=O)NCCCCCOCCOCCCCCOCC(F)CNC(*)=O]

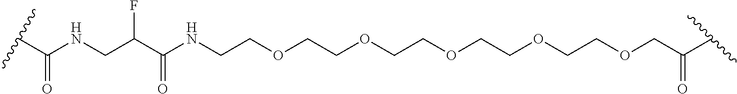 [SMILES: *C(=O)COCCOCCOCCOCCOCCNC(=O)C(F)CNC(*)=O]

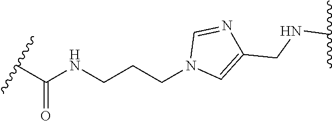 [SMILES: *NCc1cn(CCCNC(*)=O)cn1]

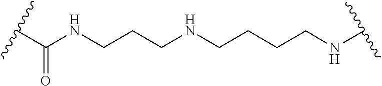 [SMILES: *NCCCCNCCCNC(*)=O]

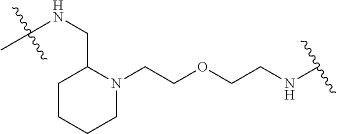 [SMILES: *NCCOCCN1CCCCC1CN*]

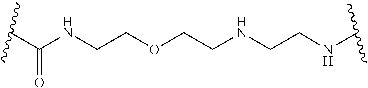 [SMILES: *NCCNCCOCCNC(*)=O]

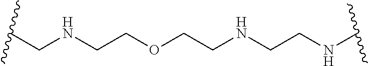 [SMILES: *CNCCOCCNCCN*]

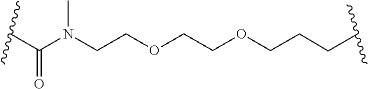 [SMILES: *CCCOCCOCCN(C)C(*)=O]

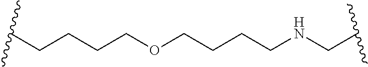 [SMILES: *CCCCOCCCCNC*]

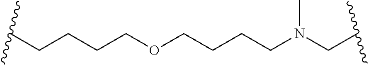 [SMILES: *CCCCOCCCCN(C)C*]

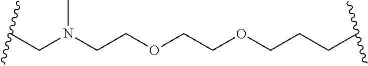 [SMILES: *CCCOCCOCCN(C)C*]

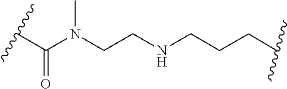 [SMILES: *CCCNCCN(C)C(*)=O]

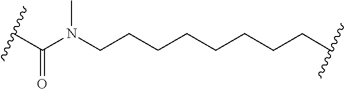 [SMILES: *CCCCCCCCN(C)C(*)=O]

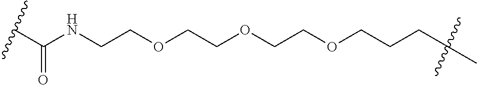 [SMILES: *CCCOCCOCCOCCNC(*)=O]

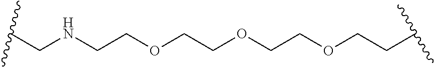 [SMILES: *CCOCCOCCOCCNC*]

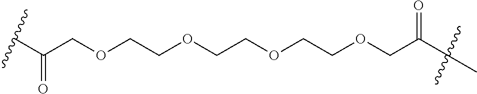 [SMILES: *C(=O)COCCOCCOCCOCC(*)=O]

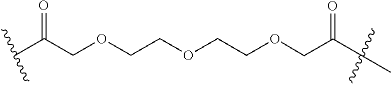 [SMILES: *C(=O)COCCOCCOCC(*)=O]

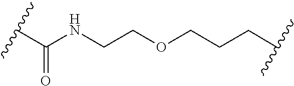 [SMILES: *CCCOCCNC(*)=O]

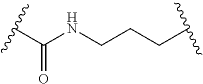 [SMILES: *CCCNC(*)=O]

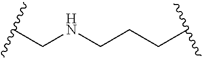 [SMILES: *CCCNC*]

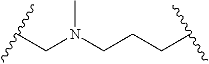 [SMILES: *CCCN(C)C*]

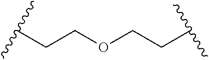 [SMILES: *CCOCC*]

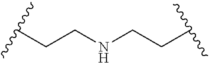 [SMILES: *CCNCC*]

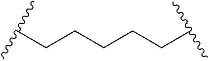 [SMILES: *CCCCC*]

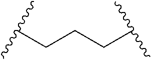 [SMILES: *CCC*]

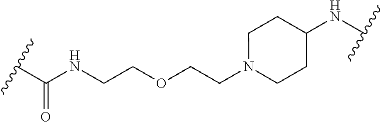 [SMILES: *NC1CCN(CCOCCNC(*)=O)CC1]

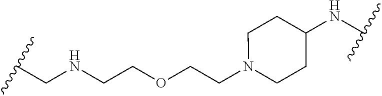 [SMILES: *CNCCOCCN1CCC(N*)CC1]

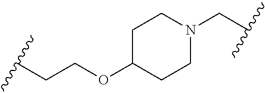 [SMILES: *CCOC1CCN(C*)CC1]

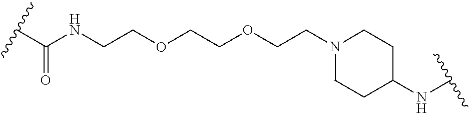 [SMILES: *NC1CCN(CCOCCOCCNC(*)=O)CC1]

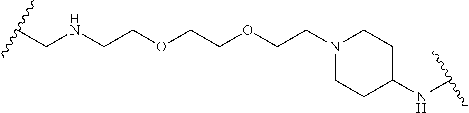 [SMILES: *CNCCOCCOCCN1CCC(N*)CC1]

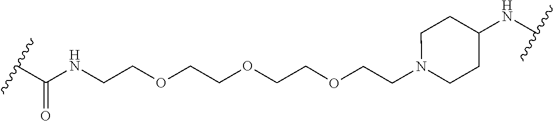 [SMILES: *NC1CCN(CCOCCOCCOCCNC(*)=O)CC1]

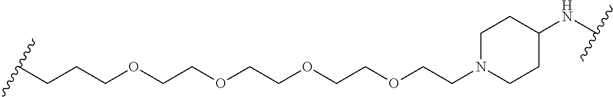 [SMILES: *CCCOCCOCCOCCOCCN1CCC(N*)CC1]

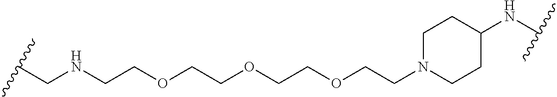 [SMILES: *CNCCOCCOCCOCCN1CCC(N*)CC1]

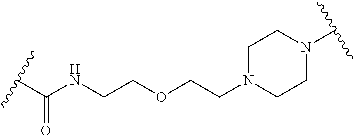 [SMILES: *C(=O)NCCOCCN1CCN(*)CC1]

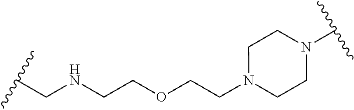 [SMILES: *CNCCOCCN1CCN(*)CC1]

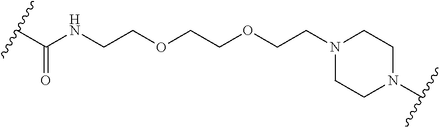 [SMILES: *C(=O)NCCOCCOCCN1CCN(*)CC1]

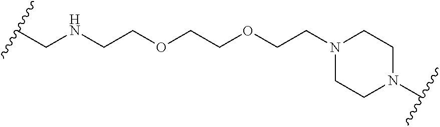 [SMILES: *CNCCOCCOCCN1CCN(*)CC1]

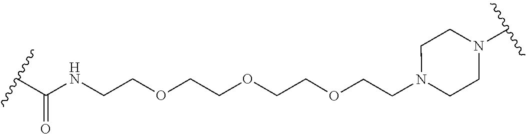 [SMILES: *C(=O)NCCOCCOCCOCCN1CCN(*)CC1]

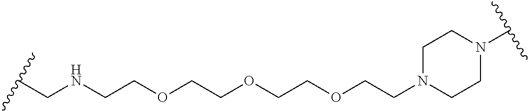 [SMILES: *CNCCOCCOCCOCCN1CCN(*)CC1]

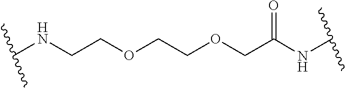 [SMILES: *NCCOCCOCC(=O)N*]

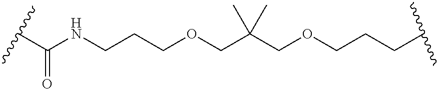 [SMILES: *CCCOCC(C)(C)COCCCNC(*)=O]

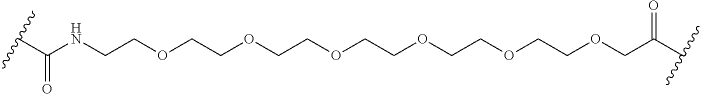 [SMILES: *C(=O)COCCOCCOCCOCCOCCOCCNC(*)=O]

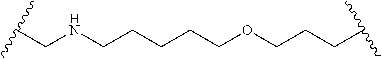 [SMILES: *CCCOCCCCCNC*]

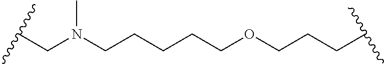 [SMILES: *CCCOCCCCCN(C)C*]

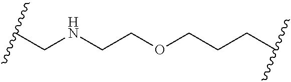 [SMILES: *CCCOCCNC*]

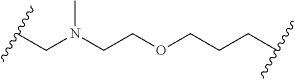 [SMILES: *CCCOCCN(C)C*]

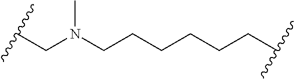 [SMILES: *CCCCCCN(C)C*]

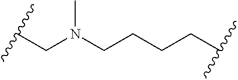 [SMILES: *CCCCN(C)C*]

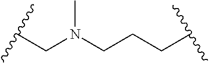 [SMILES: *CCCN(C)C*]

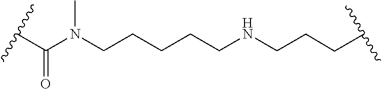 [SMILES: *CCCNCCCCCN(C)C(*)=O]

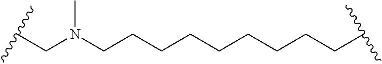 [SMILES: *CCCCCCCCCN(C)C*]

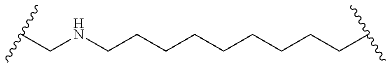 [SMILES: *CCCCCCCCCNC*]

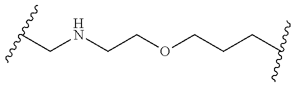 [SMILES: *CCCOCCNC*]

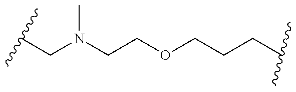 [SMILES: *CCCOCCN(C)C*]

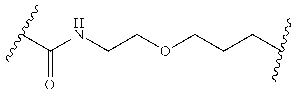 [SMILES: *CCCOCCNC(*)=O]

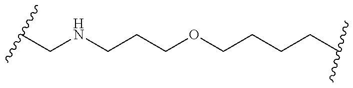 [SMILES: *CCCCOCCCNC*]

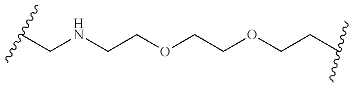 [SMILES: *CCOCCOCCNC*]

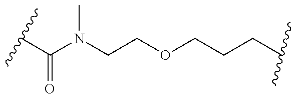 [SMILES: *CCCOCCN(C)C(*)=O]

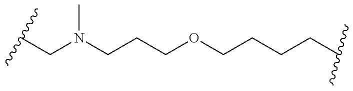 [SMILES: *CCCCOCCCN(C)C*]

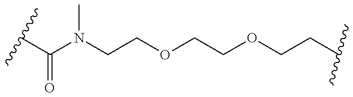 [SMILES: *CCOCCOCCN(C)C(*)=O]

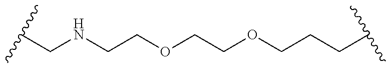 [SMILES: *CCCOCCOCCNC*]

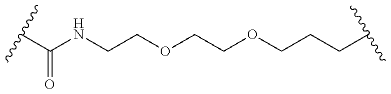 [SMILES: *CCCOCCOCCNC(*)=O]

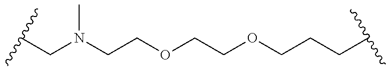 [SMILES: *CCCOCCOCCN(C)C*]

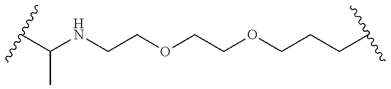 [SMILES: *CCCOCCOCCNC(*)C]

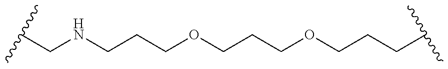 [SMILES: *CCCOCCCOCCCNC*]

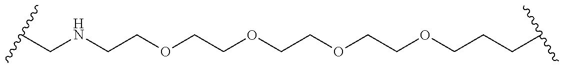 [SMILES: *CCCOCCOCCOCCOCCNC*]

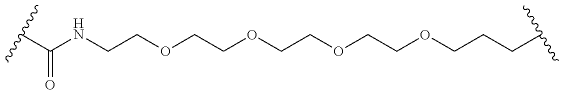 [SMILES: *CCCOCCOCCOCCOCCNC(*)=O]

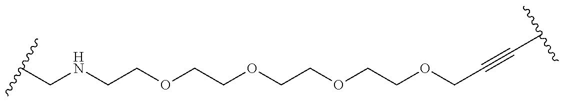 [SMILES: *C#CCOCCOCCOCCOCCNC*]

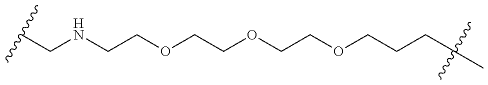 [SMILES: *CCCOCCOCCOCCNC*]

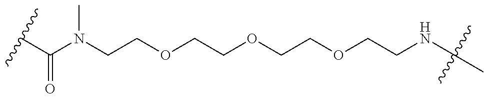 [SMILES: *NCCOCCOCCOCCN(C)C(*)=O]

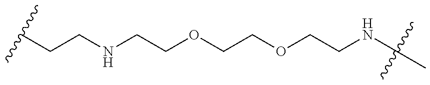 [SMILES: *CCNCCOCCOCCN*]

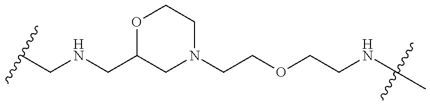 [SMILES: *CNCC1CN(CCOCCN*)CCO1]

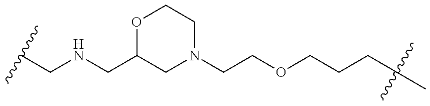 [SMILES: *CCCOCCN1CCOC(CNC*)C1]

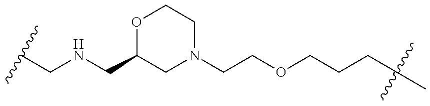 [SMILES: *CCCOCCN1CCO[C@H](CNC*)C1]

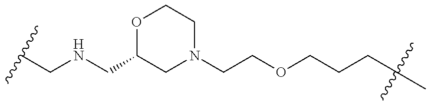 [SMILES: *CCCOCCN1CCO[C@@H](CNC*)C1]

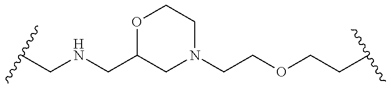 [SMILES: *CCOCCN1CCOC(CNC*)C1]

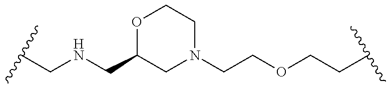 [SMILES: *CCOCCN1CCO[C@H](CNC*)C1]

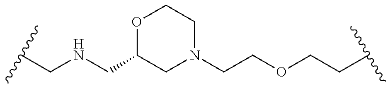 [SMILES: *CCOCCN1CCO[C@@H](CNC*)C1]

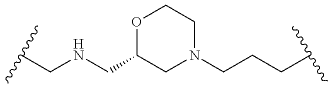 [SMILES: *CCCN1CCO[C@@H](CNC*)C1]

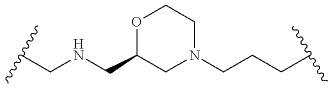 [SMILES: *CCCN1CCO[C@H](CNC*)C1]

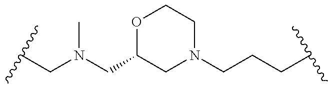 [SMILES: *CCCN1CCO[C@@H](CN(C)C*)C1]

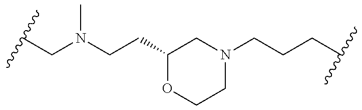 [SMILES: *CCCN1CCO[C@H](CCN(C)C*)C1]

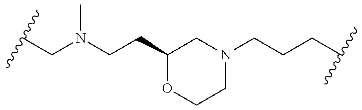 [SMILES: *CCCN1CCO[C@@H](CCN(C)C*)C1]

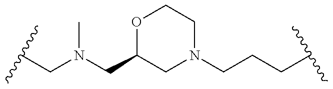 [SMILES: *CCCN1CCO[C@H](CN(C)C*)C1]

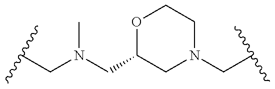 [SMILES: *CN(C)C[C@H]1CN(C*)CCO1]

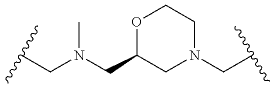 [SMILES: *CN(C)C[C@@H]1CN(C*)CCO1]

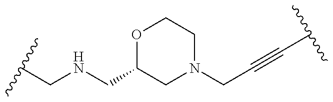 [SMILES: *C#CCN1CCO[C@@H](CNC*)C1]

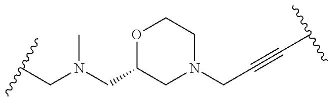 [SMILES: *C#CCN1CCO[C@@H](CN(C)C*)C1]

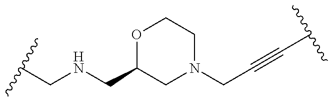 [SMILES: *C#CCN1CCO[C@H](CNC*)C1]

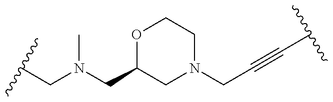 [SMILES: *C#CCN1CCO[C@H](CN(C)C*)C1]

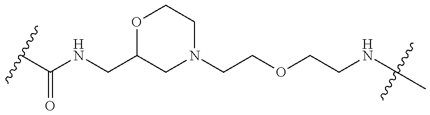 [SMILES: *NCCOCCN1CCOC(CNC(*)=O)C1]

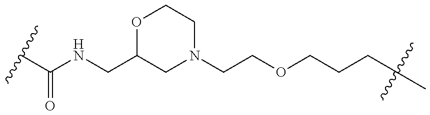 [SMILES: *CCCOCCN1CCOC(CNC(*)=O)C1]

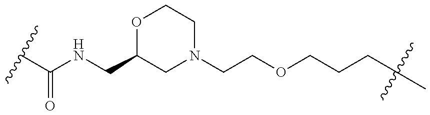 [SMILES: *CCCOCCN1CCO[C@H](CNC(*)=O)C1]

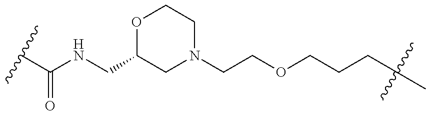 [SMILES: *CCCOCCN1CCO[C@@H](CNC(*)=O)C1]

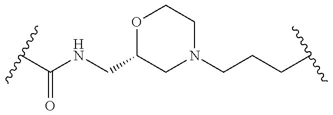 [SMILES: *CCCN1CCO[C@@H](CNC(*)=O)C1]

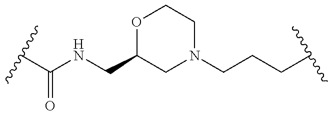 [SMILES: *CCCN1CCO[C@H](CNC(*)=O)C1]

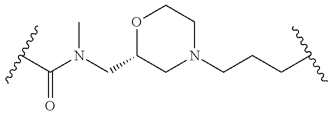 [SMILES: *CCCN1CCO[C@@H](CN(C)C(*)=O)C1]

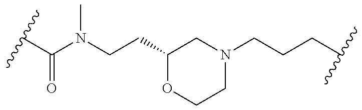 [SMILES: *CCCN1CCO[C@H](CCN(C)C(*)=O)C1]

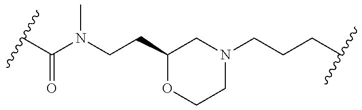 [SMILES: *CCCN1CCO[C@@H](CCN(C)C(*)=O)C1]

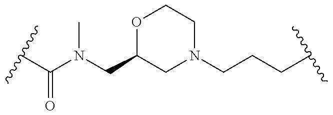 [SMILES: *CCCN1CCO[C@H](CN(C)C(*)=O)C1]

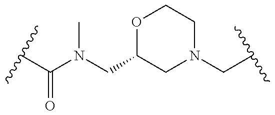 [SMILES: *CN1CCO[C@@H](CN(C)C(*)=O)C1]

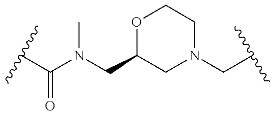 [SMILES: *CN1CCO[C@H](CN(C)C(*)=O)C1]

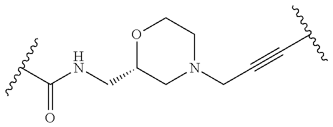 [SMILES: *C#CCN1CCO[C@@H](CNC(*)=O)C1]

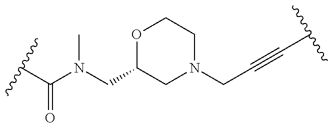 [SMILES: *C#CCN1CCO[C@@H](CN(C)C(*)=O)C1]

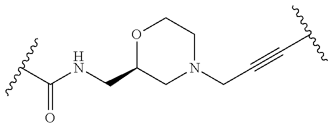 [SMILES: *C#CCN1CCO[C@H](CNC(*)=O)C1]

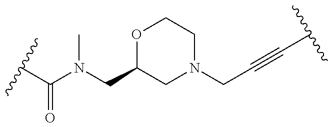 [SMILES: *C#CCN1CCO[C@H](CN(C)C(*)=O)C1]

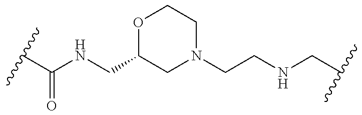 [SMILES: *CNCCN1CCO[C@@H](CNC(*)=O)C1]

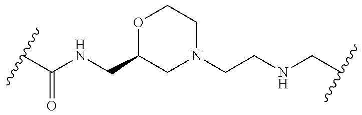 [SMILES: *CNCCN1CCO[C@H](CNC(*)=O)C1]

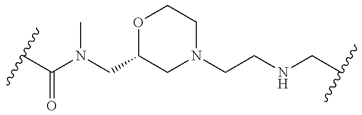 [SMILES: *CNCCN1CCO[C@@H](CN(C)C(*)=O)C1]

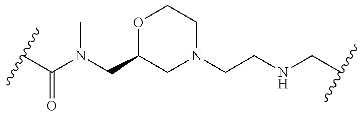 [SMILES: *CNCCN1CCO[C@H](CN(C)C(*)=O)C1]

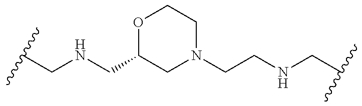 [SMILES: *CNCCN1CCO[C@@H](CNC*)C1]

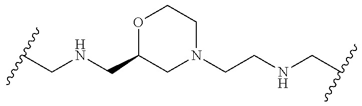 [SMILES: *CNCCN1CCO[C@H](CNC*)C1]

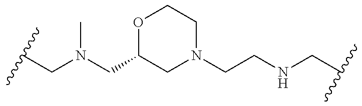 [SMILES: *CNCCN1CCO[C@@H](CN(C)C*)C1]

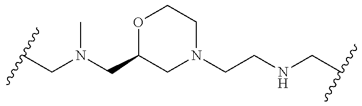 [SMILES: *CNCCN1CCO[C@H](CN(C)C*)C1]

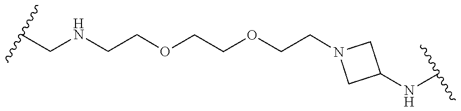 [SMILES: *CNCCOCCOCCN1CC(N*)C1]

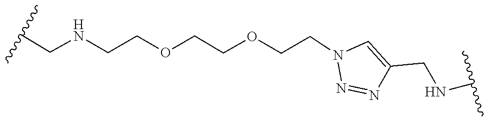 [SMILES: *CNCCOCCOCCn1cc(CN*)nn1]

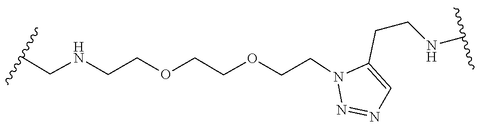 [SMILES: *CNCCOCCOCCn1nncc1CCN*]

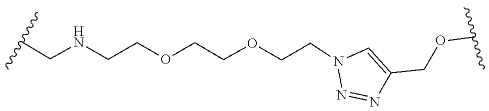 [SMILES: *CNCCOCCOCCn1cc(CO*)nn1]

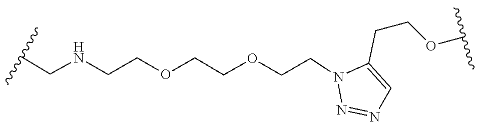 [SMILES: *CNCCOCCOCCn1nncc1CCO*]

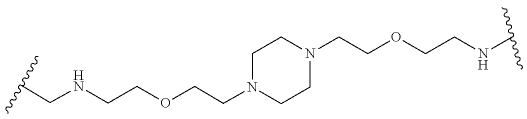 [SMILES: *CNCCOCCN1CCN(CCOCCN*)CC1]

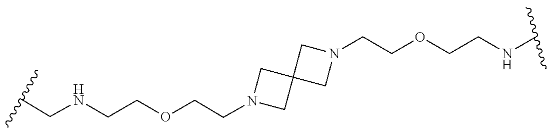 [SMILES: *CNCCOCCN1CC2(CN(CCOCCN*)C2)C1]

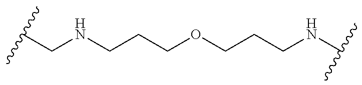 [SMILES: *CNCCCOCCCN*]

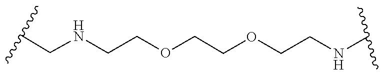 [SMILES: *CNCCOCCOCCN*]

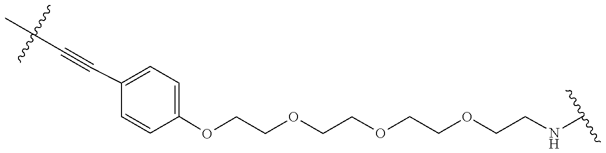 [SMILES: *C#Cc1ccc(OCCOCCOCCOCCN*)cc1]

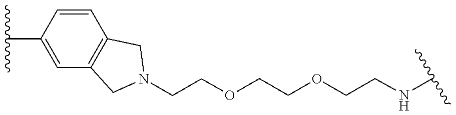 [SMILES: *NCCOCCOCCN1Cc2ccc(*)cc2C1]

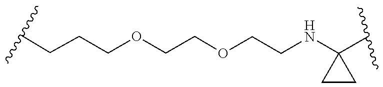 [SMILES: *CCCOCCOCCNC1(*)CC1]

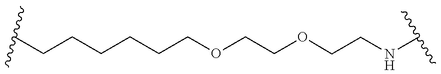 [SMILES: *CCCCCCOCCOCCN*]

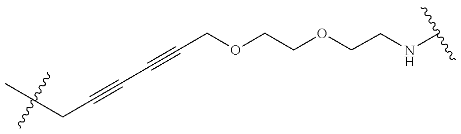 [SMILES: *CC#CC#CCOCCOCCN*]

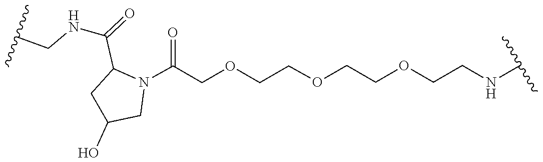 [SMILES: *CNC(=O)C1CC(O)CN1C(=O)COCCOCCOCCN*]

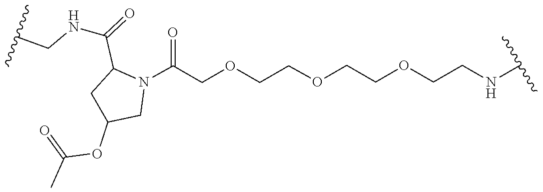 [SMILES: *CNC(=O)C1CC(OC(C)=O)CN1C(=O)COCCOCCOCCN*]

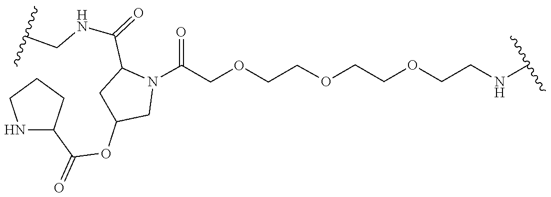 [SMILES: *CNC(=O)C1CC(OC(=O)C2CCCN2)CN1C(=O)COCCOCCOCCN*]

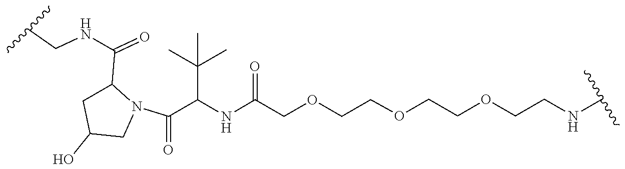 [SMILES: *CNC(=O)C1CC(O)CN1C(=O)C(NC(=O)COCCOCCOCCN*)C(C)(C)C]

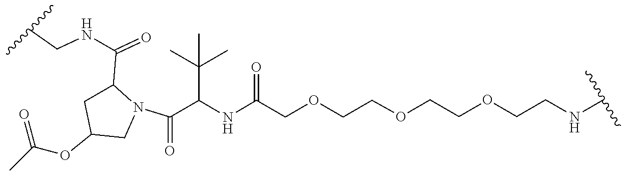 [SMILES: *CNC(=O)C1CC(OC(C)=O)CN1C(=O)C(NC(=O)COCCOCCOCCN*)C(C)(C)C]

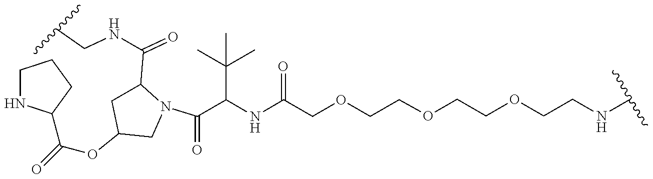 [SMILES: *CNC(=O)C1CC(OC(=O)C2CCCN2)CN1C(=O)C(NC(=O)COCCOCCOCCN*)C(C)(C)C]

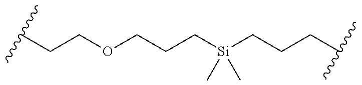 [SMILES: *CCC[Si](C)(C)CCCOCC*]

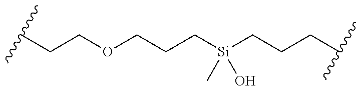 [SMILES: *CCC[Si](C)(O)CCCOCC*]

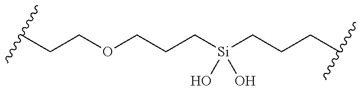 [SMILES: *CCC[Si](O)(O)CCCOCC*]

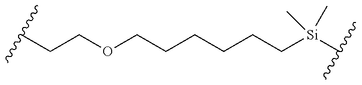 [SMILES: *CCOCCCCCC[Si](*)(C)C]

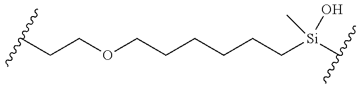 [SMILES: *CCOCCCCCC[Si](*)(C)O]

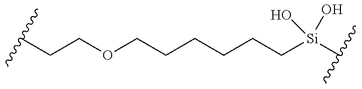 [SMILES: *CCOCCCCCC[Si](*)(O)O]

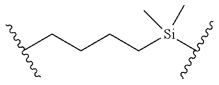 [SMILES: *CCCC[Si](*)(C)C]

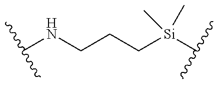 [SMILES: *NCCC[Si](*)(C)C]

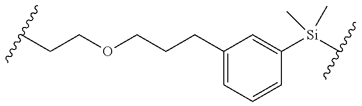 [SMILES: *CCOCCCc1cccc([Si](*)(C)C)c1]

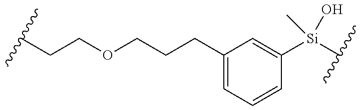 [SMILES: *CCOCCCc1cccc([Si](*)(C)O)c1]

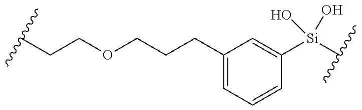 [SMILES: *CCOCCCc1cccc([Si](*)(O)O)c1]

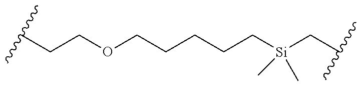 [SMILES: *CCOCCCCC[Si](C)(C)C*]

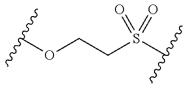 [SMILES: *OCCS(*)(=O)=O]

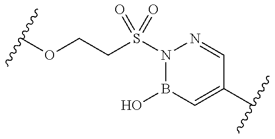 [SMILES: *OCCS(=O)(=O)N1N=CC(*)=CB1O]

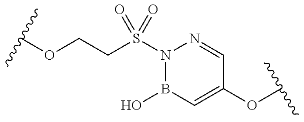 [SMILES: *OCCS(=O)(=O)N1N=CC(O*)=CB1O]

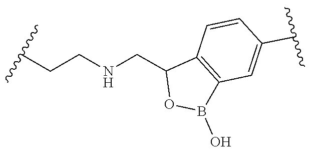 [SMILES: *CCNCC1OB(O)c2cc(*)ccc21]

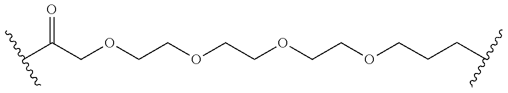 [SMILES: *CCCOCCOCCOCCOCC(*)=O]

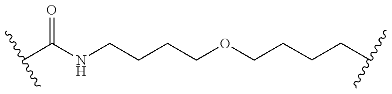 [SMILES: *CCCCOCCCCNC(*)=O]

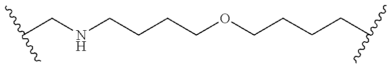 [SMILES: *CCCCOCCCCNC*]

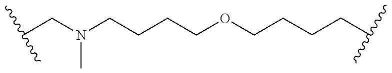 [SMILES: *CCCCOCCCCN(C)C*]

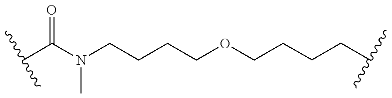 [SMILES: *CCCCOCCCCN(C)C(*)=O]

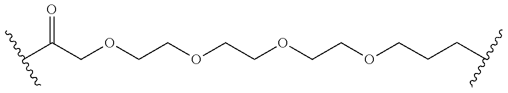 [SMILES: *CCCOCCOCCOCCOCC(*)=O]

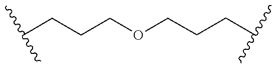 [SMILES: *CCCOCCC*]

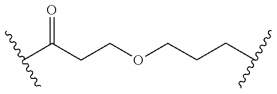 [SMILES: *CCCOCCC(*)=O]

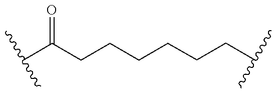 [SMILES: *CCCCCCC(*)=O]

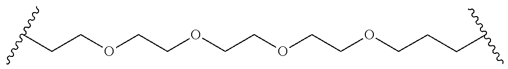 [SMILES: *CCCOCCOCCOCCOCC*]

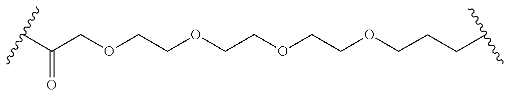 [SMILES: *CCCOCCOCCOCCOCC(*)=O]

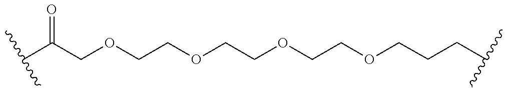 [SMILES: *CCCOCCOCCOCCOCC(*)=O]

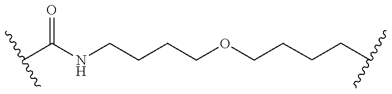 [SMILES: *CCCCOCCCCNC(*)=O]

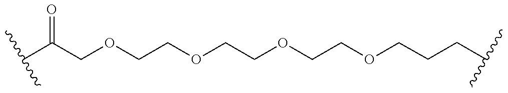 [SMILES: *CCCOCCOCCOCCOCC(*)=O]

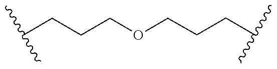 [SMILES: *CCCOCCC*]

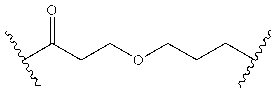 [SMILES: *CCCOCCC(*)=O]

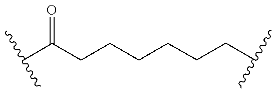 [SMILES: *CCCCCCC(*)=O]

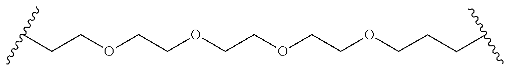 [SMILES: *CCCOCCOCCOCCOCC*]

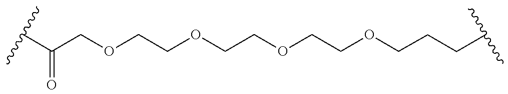 [SMILES: *CCCOCCOCCOCCOCC(*)=O]

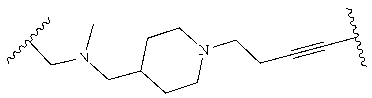 [SMILES: *C#CCCN1CCC(CN(C)C*)CC1]

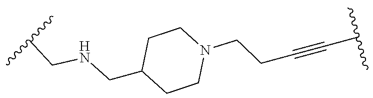 [SMILES: *C#CCCN1CCC(CNC*)CC1]

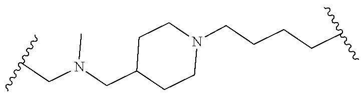 [SMILES: *CCCCN1CCC(CN(C)C*)CC1]

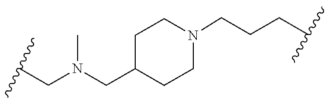 [SMILES: *CCCN1CCC(CN(C)C*)CC1]

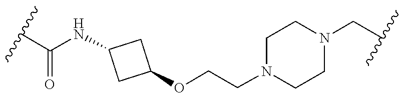 [SMILES: *CN1CCN(CCO[C@H]2C[C@H](NC(*)=O)C2)CC1]

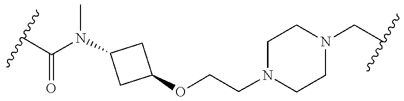 [SMILES: *CN1CCN(CCO[C@H]2C[C@H](N(C)C(*)=O)C2)CC1]

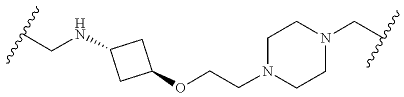 [SMILES: *CN[C@H]1C[C@H](OCCN2CCN(C*)CC2)C1]

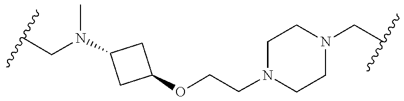 [SMILES: *CN1CCN(CCO[C@H]2C[C@H](N(C)C*)C2)CC1]

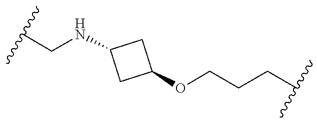 [SMILES: *CCCO[C@H]1C[C@H](NC*)C1]

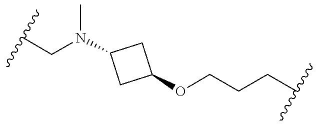 [SMILES: *CCCO[C@H]1C[C@H](N(C)C*)C1]

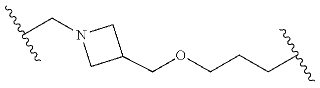 [SMILES: *CCCOCC1CN(C*)C1]

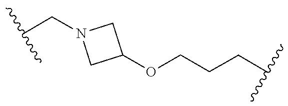 [SMILES: *CCCOC1CN(C*)C1]

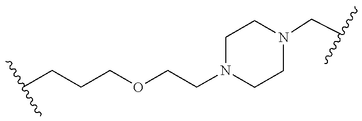 [SMILES: *CCCOCCN1CCN(C*)CC1]

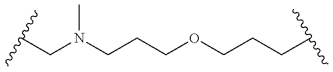 [SMILES: *CCCOCCCN(C)C*]

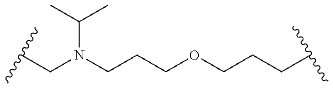 [SMILES: *CCCOCCCN(C*)C(C)C]

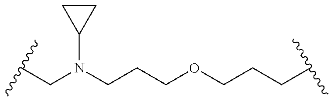 [SMILES: *CCCOCCCN(C*)C1CC1]

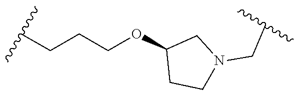 [SMILES: *CCCO[C@@H]1CCN(C*)C1]

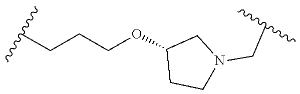 [SMILES: *CCCO[C@H]1CCN(C*)C1]

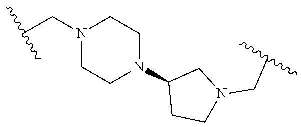 [SMILES: *CN1CCN([C@@H]2CCN(C*)C2)CC1]

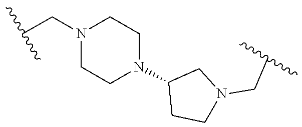 [SMILES: *CN1CCN([C@H]2CCN(C*)C2)CC1]

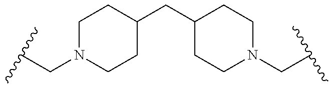 [SMILES: *CN1CCC(CC2CCN(C*)CC2)CC1]

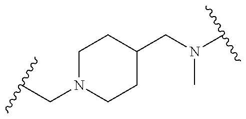 [SMILES: *CN1CCC(CN(*)C)CC1]

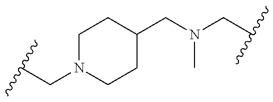 [SMILES: *CN(C)CC1CCN(C*)CC1]

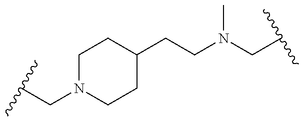 [SMILES: *CN(C)CCC1CCN(C*)CC1]

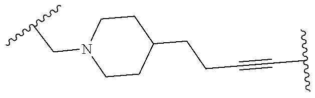 [SMILES: *C#CCCC1CCN(C*)CC1]

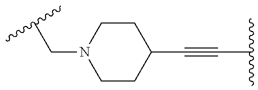 [SMILES: *C#CC1CCN(C*)CC1]

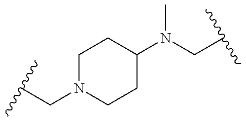 [SMILES: *CN1CCC(N(C)C*)CC1]

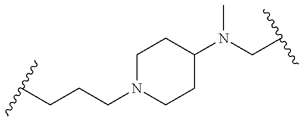 [SMILES: *CCCN1CCC(N(C)C*)CC1]

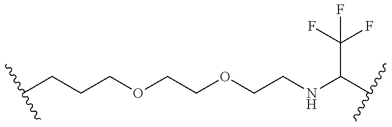 [SMILES: *CCCOCCOCCNC(*)C(F)(F)F]

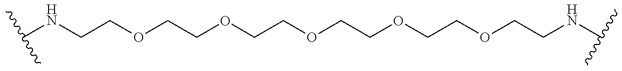 [SMILES: *NCCOCCOCCOCCOCCOCCN*]

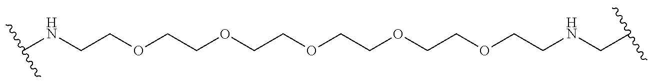 [SMILES: *CNCCOCCOCCOCCOCCOCCN*]

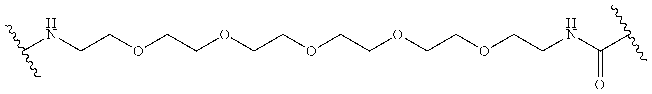 [SMILES: *NCCOCCOCCOCCOCCOCCNC(*)=O]

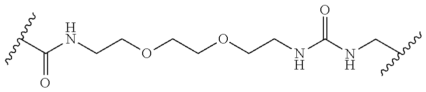 [SMILES: *CNC(=O)NCCOCCOCCNC(*)=O]

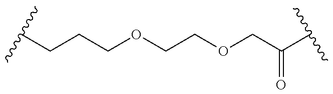 [SMILES: *CCCOCCOCC(*)=O]

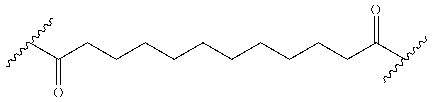 [SMILES: *C(=O)CCCCCCCCCCC(*)=O]

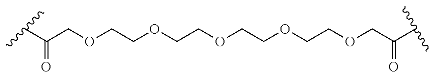 [SMILES: *C(=O)COCCOCCOCCOCCOCC(*)=O]

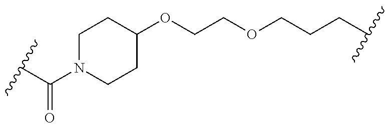 [SMILES: *CCCOCCOC1CCN(C(*)=O)CC1]

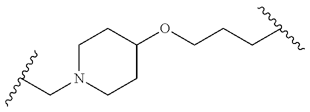 [SMILES: *CCCOC1CCN(C*)CC1]

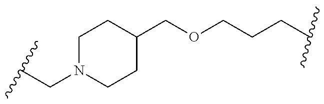 [SMILES: *CCCOCC1CCN(C*)CC1]

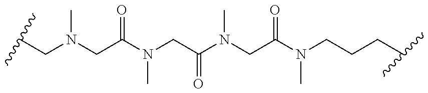 [SMILES: *CCCN(C)C(=O)CN(C)C(=O)CN(C)C(=O)CN(C)C*]

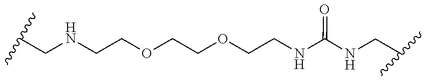 [SMILES: *CNCCOCCOCCNC(=O)NC*]

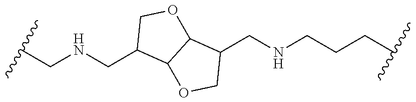 [SMILES: *CCCNCC1COC2C(CNC*)COC12]

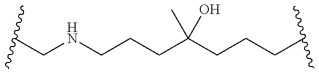 [SMILES: *CCCC(C)(O)CCCNC*]

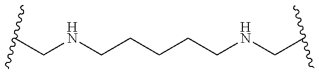 [SMILES: *CNCCCCCNC*]

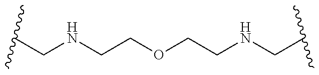 [SMILES: *CNCCOCCNC*]

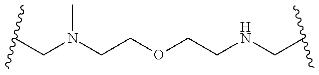 [SMILES: *CNCCOCCN(C)C*]

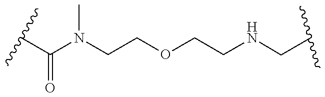 [SMILES: *CNCCOCCN(C)C(*)=O]

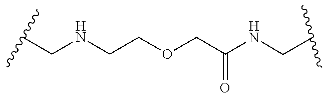 [SMILES: *CNCCOCC(=O)NC*]

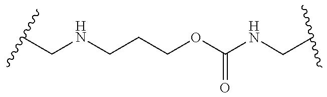 [SMILES: *CNCCCOC(=O)NC*]

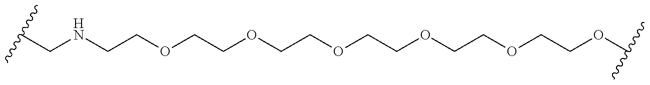 [SMILES: *CNCCOCCOCCOCCOCCOCCO*]

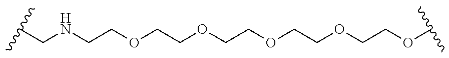 [SMILES: *CNCCOCCOCCOCCOCCO*]

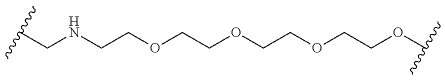 [SMILES: *CNCCOCCOCCOCCO*]

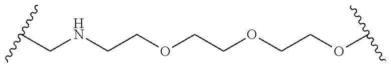 [SMILES: *CNCCOCCOCCO*]

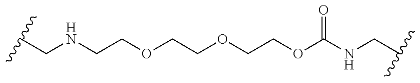 [SMILES: *CNCCOCCOCCOC(=O)NC*]

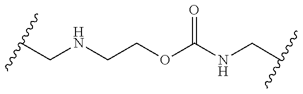 [SMILES: *CNCCOC(=O)NC*]

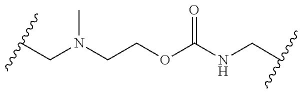 [SMILES: *CNC(=O)OCCN(C)C*]

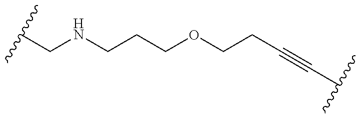 [SMILES: *C#CCCOCCCNC*]

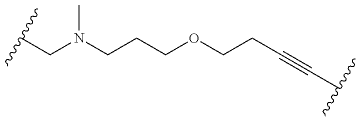 [SMILES: *C#CCCOCCCN(C)C*]

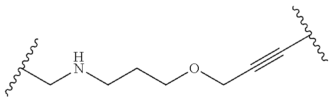 [SMILES: *C#CCOCCCNC*]

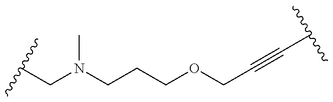 [SMILES: *C#CCOCCCN(C)C*]

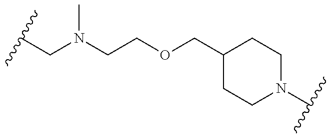 [SMILES: *CN(C)CCOCC1CCN(*)CC1]

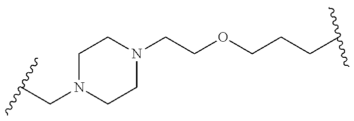 [SMILES: *CCCOCCN1CCN(C*)CC1]

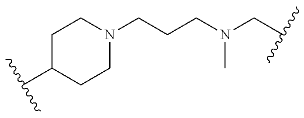 [SMILES: *CN(C)CCCN1CCC(*)CC1]

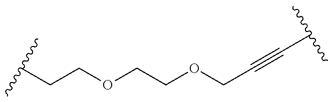 [SMILES: *C#CCOCCOCC*]

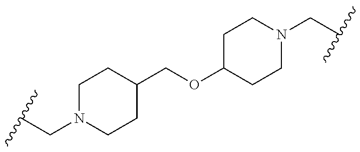 [SMILES: *CN1CCC(COC2CCN(C*)CC2)CC1]

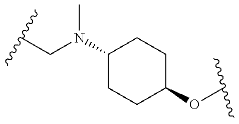 [SMILES: *CN(C)[C@H]1CC[C@H](O*)CC1]

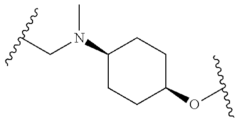 [SMILES: *CN(C)[C@H]1CC[C@@H](O*)CC1]

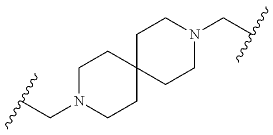 [SMILES: *CN1CCC2(CC1)CCN(C*)CC2]

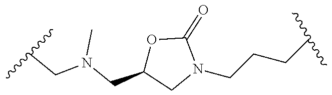 [SMILES: *CCCN1C[C@@H](CN(C)C*)OC1=O]

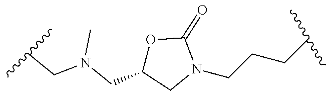 [SMILES: *CCCN1C[C@H](CN(C)C*)OC1=O]

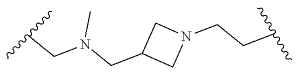 [SMILES: *CCN1CC(CN(C)C*)C1]

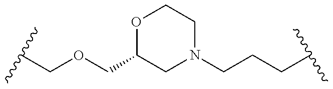 [SMILES: *CCCN1CCO[C@@H](COC*)C1]

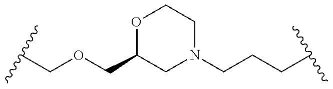 [SMILES: *CCCN1CCO[C@H](COC*)C1]

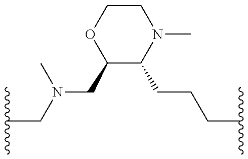 [SMILES: *CCC[C@@H]1[C@@H](CN(C)C*)OCCN1C]

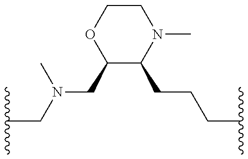 [SMILES: *CCC[C@H]1[C@@H](CN(C)C*)OCCN1C]

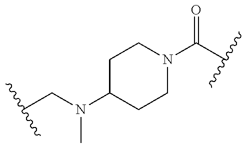 [SMILES: *CN(C)C1CCN(C(*)=O)CC1]

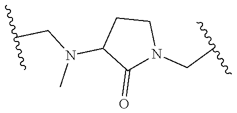 [SMILES: *CN1CCC(N(C)C*)C1=O]

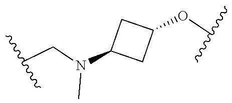 [SMILES: *CN(C)[C@H]1C[C@H](O*)C1]

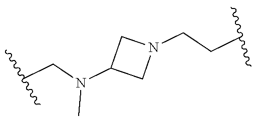 [SMILES: *CCN1CC(N(C)C*)C1]

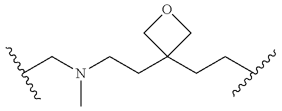 [SMILES: *CCC1(CCN(C)C*)COC1]

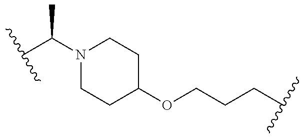 [SMILES: *CCCOC1CCN([C@@H](*)C)CC1]

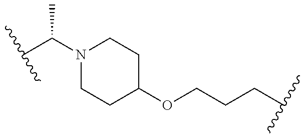 [SMILES: *CCCOC1CCN([C@H](*)C)CC1]

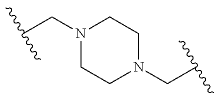 [SMILES: *CN1CCN(C*)CC1]

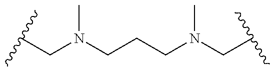 [SMILES: *CN(C)CCCN(C)C*]

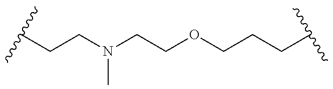 [SMILES: *CCCOCCN(C)CC*]

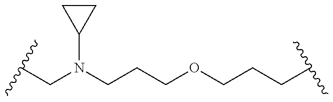 [SMILES: *CCCOCCCN(C*)C1CC1]

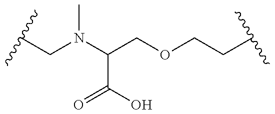 [SMILES: *CCOCC(C(=O)O)N(C)C*]

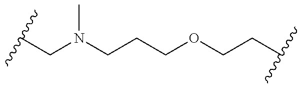 [SMILES: *CCOCCCN(C)C*]

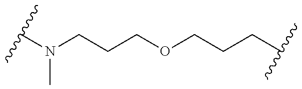 [SMILES: *CCCOCCCN(*)C]

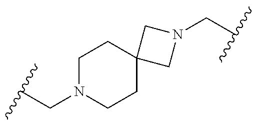 [SMILES: *CN1CCC2(CC1)CN(C*)C2]

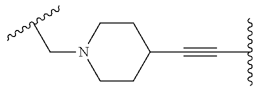 [SMILES: *C#CC1CCN(C*)CC1]

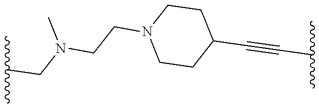 [SMILES: *C#CC1CCN(CCN(C)C*)CC1]

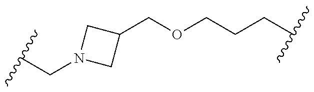 [SMILES: *CCCOCC1CN(C*)C1]

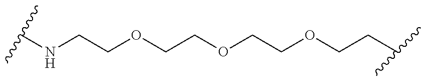 [SMILES: *CCOCCOCCOCCN*]

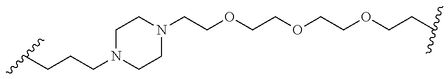 [SMILES: *CCCN1CCN(CCOCCOCCOCC*)CC1]

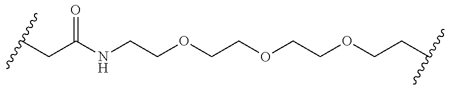 [SMILES: *CCOCCOCCOCCNC(=O)C*]

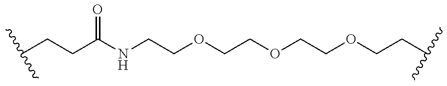 [SMILES: *CCOCCOCCOCCNC(=O)CC*]

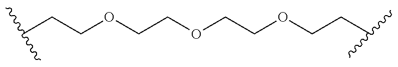 [SMILES: *CCOCCOCCOCC*]

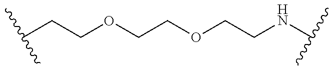 [SMILES: *CCOCCOCCN*]

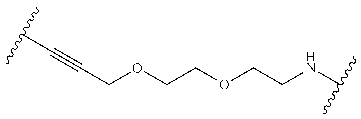 [SMILES: *C#CCOCCOCCN*]

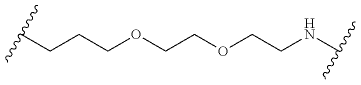 [SMILES: *CCCOCCOCCN*]

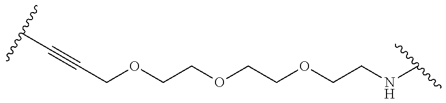 [SMILES: *C#CCOCCOCCOCCN*]

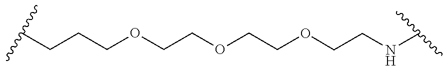 [SMILES: *CCCOCCOCCOCCN*]

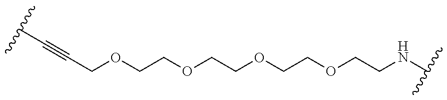 [SMILES: *C#CCOCCOCCOCCOCCN*]

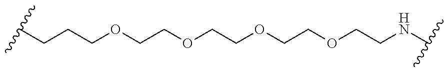 [SMILES: *CCCOCCOCCOCCOCCN*]

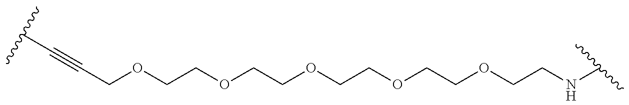 [SMILES: *C#CCOCCOCCOCCOCCOCCN*]

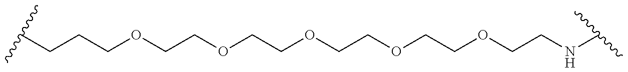 [SMILES: *CCCOCCOCCOCCOCCOCCN*]

In some embodiments, L is selected from those depicted in Table 2, above.

In some embodiments, the compound is not selected from one of those described in WO 2019/209975.

In some embodiments, the compound of Formula A is not selected from the following:

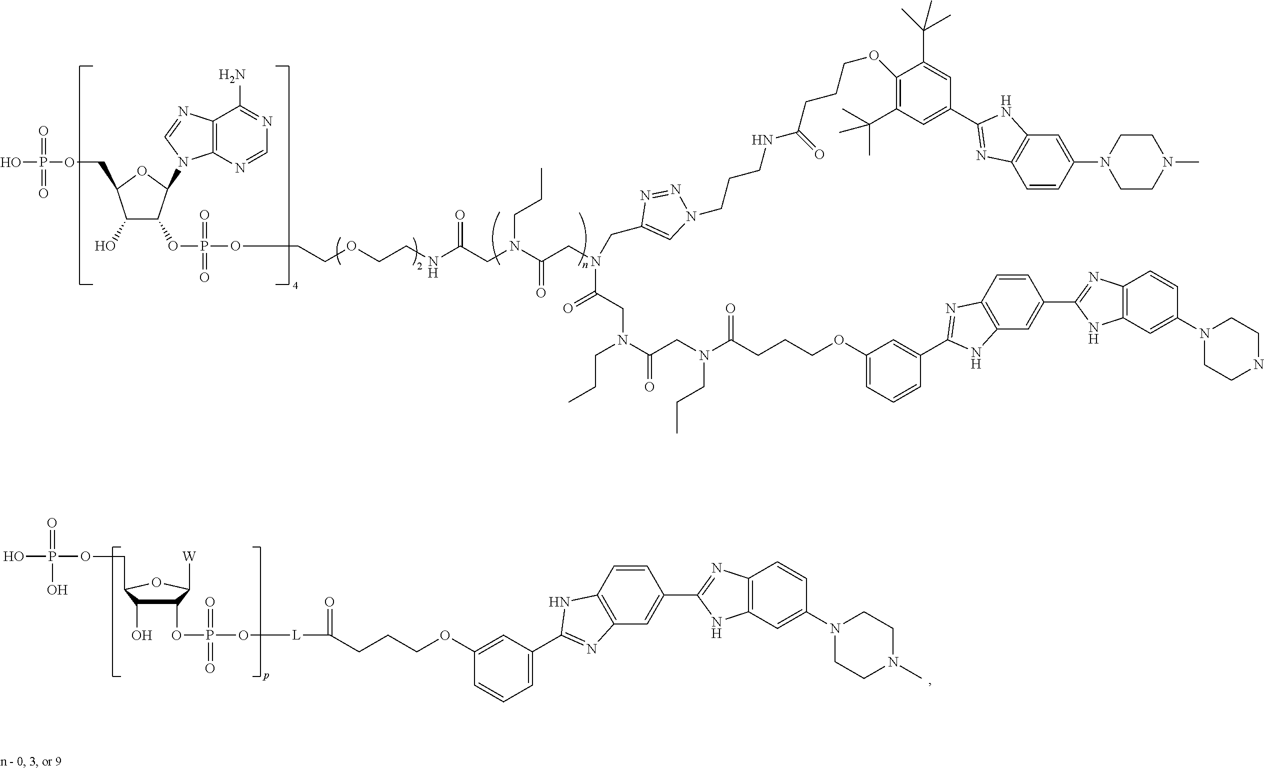

wherein W is a nucleobase; L is a linker moiety, and p is 1 to 5.

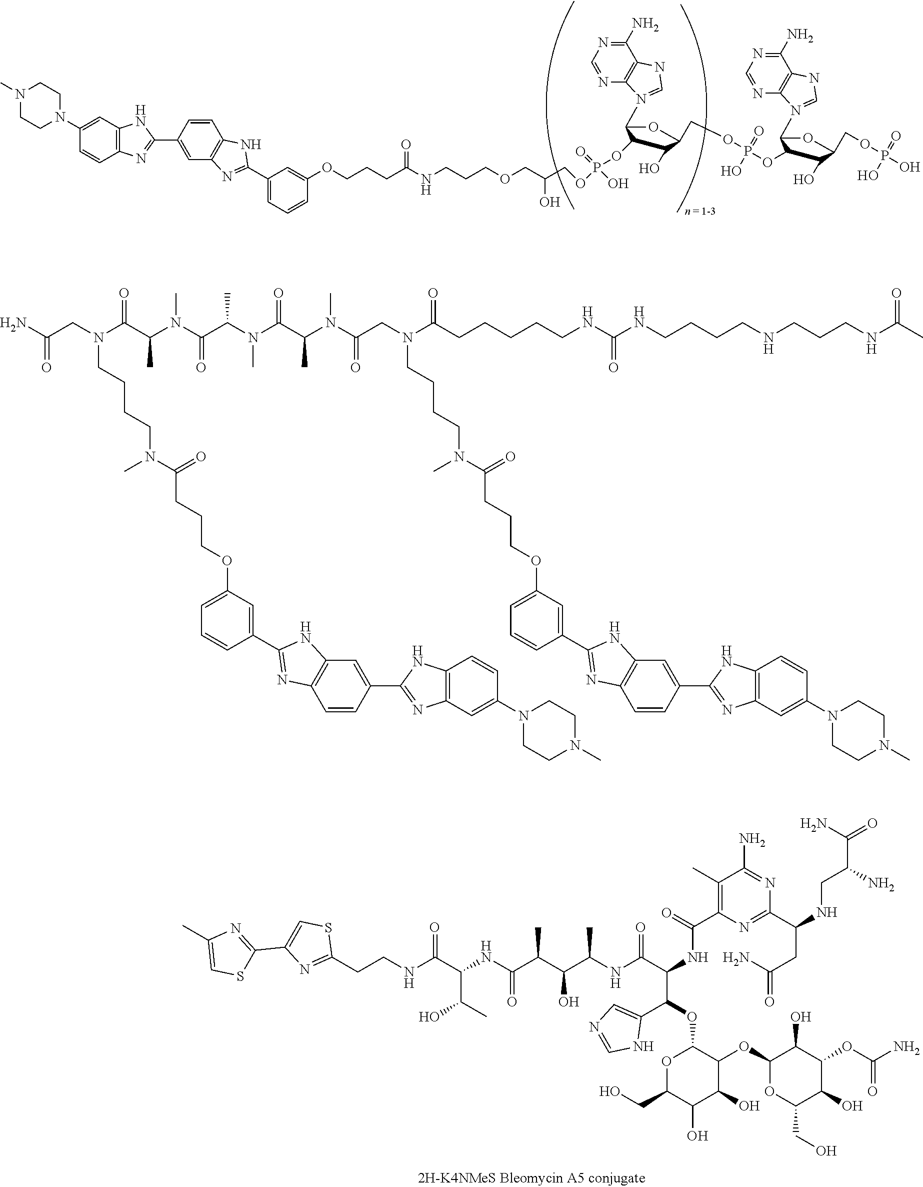

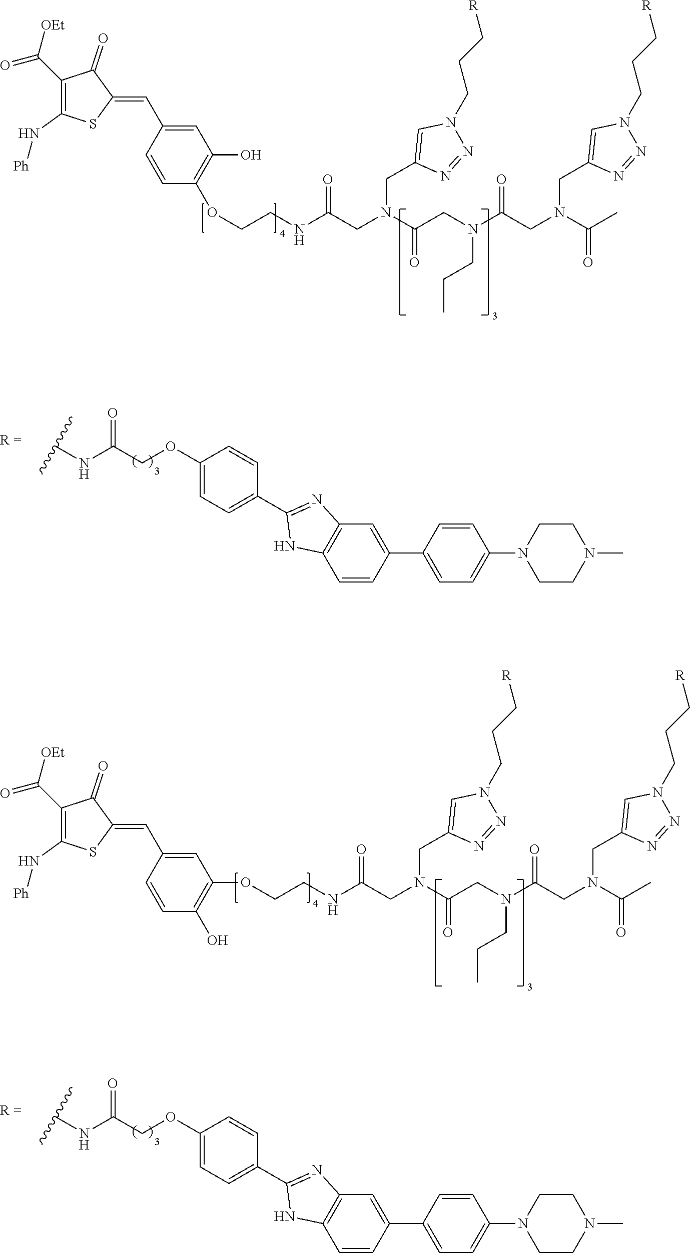

### Definitions

As used herein, a “nucleoside” refers to a molecule consisting of a guanine (G), adenine (A), thymine (T), uridine (U), or cytidine (C) base covalently linked to a pentose sugar, whereas “nucleotide” or “mononucleotide” refers to a nucleoside phosphorylated at one of the hydroxyl groups of the pentose sugar. “Nucleoside” also encompasses analogs of G, A, T, C, or U and natural or non-natural nucleic acid components wherein the base, sugar, and/or phosphate backbone have been modified or replaced. Nucleoside analogs are known in the art and include those described herein. Also included are endogenous, post-transcriptionally modified nucleosides, such as methylated nucleosides.

Linear nucleic acid molecules are said to have a “5′ terminus” (5′-end) and a “3′ terminus” (3′-end) because, except with respect to adenylation (as described elsewhere herein), mononucleotides are joined in one direction via a phosphodiester linkage (or analog thereof) to make oligonucleotides, in a manner such that a phosphate (or analog thereof) on the 5′ carbon of one mononucleotide sugar is joined to an oxygen on the 3′ carbon of the sugar of its neighboring mononucleotide. Therefore, an end of an oligonucleotide is referred to as the “5′ end” if its 5′ phosphate (or analog thereof) is not linked to the oxygen of the 3′ carbon of a mononucleotide sugar, and as the “3′ end” if its 3′ oxygen is not linked to a 5′ phosphate (or analog thereof) of a subsequent mononucleotide sugar. A “terminal nucleotide,” as used herein, is the nucleotide at the end position of the 3′ or 5′ terminus. The 3′ or 5′ terminus may alternatively end in a 3′-OH or 5′-OH if the terminal nucleotide is not phosphorylated.

As used herein, the term “nucleic acid” refers to a covalently linked sequence of nucleotides in which the 3′ position of the sugar of one nucleotide is joined by a phosphodiester bond to the 5′ position of the sugar of the next nucleotide (i.e., a 3′ to 5′ phosphodiester bond), and in which the nucleotides are linked in specific sequence; i.e., a linear order of nucleotides. “Nucleic acid” includes analogs of the foregoing wherein one or more nucleotides are modified at the base, sugar, or phosphodiester. Such analogs are known in the art and include those described elsewhere herein. As used herein, “polynucleotide” or “polynucleic acid” refers to a long nucleic acid sequence (or analog thereof) of many nucleotides. For example, but without limitation, a polynucleotide (or polynucleic acid) may be greater than 60, 61-1,000, or 201-1,000, or greater than 1,000 nucleotides in length. As used herein, an “oligonucleotide” or “oligonucleic acid” is a short polynucleotide or a portion of a polynucleotide. For example, but without limitation, an oligonucleotide may be between 5-10, 10-60, or 10-200 nucleotides in length.

In some embodiments, a nucleic acid, oligonucleotide, or polynucleotide consists of, consists primarily of, or is mostly 2′-deoxyribonucleotides (DNA) or ribonucleotides (RNA). In some embodiments, an oligonucleotide consists of or comprises 2′-deoxyribonucleotides (DNA). In some embodiments, the oligonucleotide consists of or comprises ribonucleotides (RNA). In some embodiments, the oligonucleotide is a DNA-RNA hybrid, such as a DNA sequence of contiguous nucleotides linked to an RNA sequence of contiguous nucleotides, or with some regions of RNA and some regions of DNA.

As used herein, the term “RNA-mediated” in reference to RNA-mediated disorders, diseases, and/or conditions means any disease or other deleterious condition in which RNA, such as an overexpressed, underexpressed, mutant, misfolded, expanded, pathogenic, or oncogenic RNA, is known to play a role.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry: Reactions, Mechanisms, and Structure, M. B. Smith and J. March, 7^{th} Edition, John Wiley & Sons: 2013; the entire contents of each of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally, or alternatively, any substitutable nitrogen of a bridged bicyclic group is optionally substituted. Exemplary bicyclic rings include:

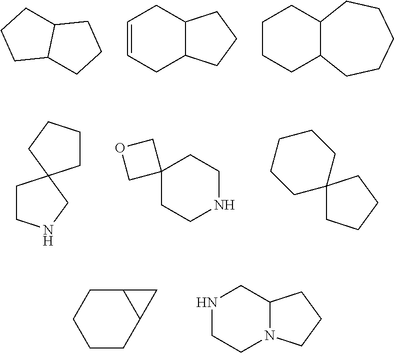

Exemplary bridged bicyclics include:

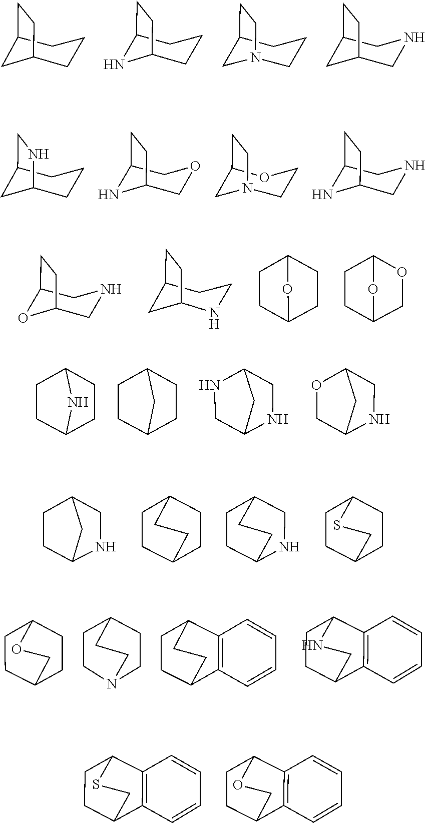

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 I electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted with a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted with a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent (“optional substituent”) at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘}) (CH₂)₀₋₄N(R^{∘}(O)R^{∘}; —N(R^{∘}C(S)R^{∘}; —(CH₂)₀₋₄ N(R^{∘})C(O)NR^{∘}₂; —N(R^{∘})C(S)NR^{∘}₂; —(CH₂)₀₋₄N(R^{∘})C(O)OR^{∘}; —N(R^{∘}N(R^{∘}C(O)R^{∘}; —N(R^{∘}N(R^{∘}C(O)NR^{∘}₂; —N(R^{∘}N(R^{∘}C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘}) R^{∘}; —C(O)C(O)R^{∘}; —C(O)CH₂C(O)R^{∘}; —C(NOR^{∘}) R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘}S(O)₂NR^{∘}₂; —N(R^{∘}S(O)₂R^{∘}; —N(OR^{∘}) R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—) N(R^{∘}₂; or —(C₁₋₄ straight or branched) alkylene) C(O)O—N(R^{∘}₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{•} together with their intervening atoms), are independently halogen, —(CH₂)₀₋₂R^{•}, (haloR^{•}, —(CH₂)₀₋₂OH, —(CH₂)₀₋₂OR^{•}, —(CH₂)₀₋₂CH(OR^{•})₂; —O(haloR^{•}, —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O) SR^{•}, —(C₁₋₄ straight or branched alkylene) C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{*}₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O) OH, —C(O) OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, C(O)CH₂C(O)R^{†}, —S(O)₂R^{†}, —S(O)₂NR^{†}₂, —C(S)NR^{†}₂, —C(NH)NR^{†}₂, or —N(R^{†}) S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl) 4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the term “binder” or “ligand” is defined as a compound that binds to a target RNA transcript or decay factor (e.g., nuclease) or RBP with measurable affinity. In certain embodiments, a binder has an IC₅₀ and/or binding constant of less than about 50 μM, less than about 1 μM, less than about 500 nM, less than about 100 nM, less than about 10 nM, or less than about 1 nM.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “RNA” (ribonucleic acid) as used herein, means a naturally-occurring or synthetic oligo- or polyribonucleotide independent of source (e.g., the RNA may be produced by a human, animal, plant, virus, or bacterium, or may be synthetic in origin), biological context (e.g., the RNA may be in the nucleus, circulating in the blood, in vitro, cell lysate, or isolated or pure form), or physical form (e.g., the RNA may be in single-, double-, or triple-stranded form (including RNA-DNA hybrids), may include epigenetic modifications, native post-transcriptional modifications, artificial modifications (e.g., obtained by chemical or in vitro modification), or other modifications, may be bound to, e.g., metal ions, small molecules, protein chaperones, or co-factors, or may be in a denatured, partially denatured, or folded state including any native or unnatural secondary or tertiary structure such as junctions (e.g., cis or trans three-way junctions (3WJ)), quadruplexes, hairpins, triplexes, hairpins, bulge loops, pseudoknots, and internal loops, etc., and any transient forms or structures adopted by the RNA). In some embodiments, the RNA is 100 or more nucleotides in length. In some embodiments, the RNA is 250 or more nucleotides in length. In some embodiments, the RNA is 350, 450, 500, 600, 750, or 1,000, 2,000, 3,000, 4,000, 5,000, 7,500, 10,000, 15,000, 25,000, 50,000, or more nucleotides in length. In some embodiments, the RNA is between 250 and 1,000 nucleotides in length. In some embodiments, the RNA is a pre-RNA, pre-miRNA, or pretranscript. In some embodiments, the RNA is a non-coding RNA (ncRNA), messenger RNA (mRNA), micro-RNA (miRNA), a ribozyme, riboswitch, lncRNA, lincRNA, snoRNA, snRNA, scaRNA, piRNA, ceRNA, pseudo-gene, viral RNA, or bacterial RNA. The term “target RNA” as used herein, means any type of RNA having or capable of adopting a secondary or tertiary structure that is capable of binding a small molecule ligand described herein. The target RNA may be inside a cell, in a cell lysate, or in isolated form prior to contacting the small molecule.

### 3. General Methods of Providing the Present Compounds

The compounds of this invention may be prepared or isolated in general by synthetic and/or semi-synthetic methods known to those skilled in the art for analogous compounds and by methods described in detail in the Examples and Figures, herein.

In the schemes and chemical reactions depicted in the detailed description, Examples, and Figures, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 7^{th} Edition, John Wiley & Sons, 2013, Comprehensive Organic Transformations, R. C. Larock, 3^{rd} Edition, John Wiley & Sons, 2018, and Protective Groups in Organic Synthesis, P. G. M. Wuts, 5^{th} edition, John Wiley & Sons, 2014, the entirety of each of which is hereby incorporated herein by reference.

As used herein, the phrase “leaving group” (LG) includes, but is not limited to, halogens (e.g., fluoride, chloride, bromide, iodide), sulfonates (e.g., mesylate, tosylate, benzenesulfonate, brosylate, nosylate, triflate), diazonium, and the like.

As used herein, the phrase “oxygen protecting group” includes, for example, carbonyl protecting groups, hydroxyl protecting groups, etc. Hydroxyl protecting groups are well known in the art and include those described in detail in Protective Groups in Organic Synthesis, P. G. M. Wuts, 5th edition, John Wiley & Sons, 2014, and Philip Kocienski, in Protecting Groups, Georg Thieme Verlag Stuttgart, New York, 1994, the entireties of which are incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio) pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl) ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protective Groups in Organic Synthesis, P. G. M. Wuts, 5^{th} edition, John Wiley & Sons, 2014, and Philip Kocienski, in Protecting Groups, Georg Thieme Verlag Stuttgart, New York, 1994, the entireties of which are incorporated herein by reference. Suitable amino protecting groups include, but are not limited to, aralkylamines, carbamates, cyclic imides, allyl amines, amides, and the like. Examples of such groups include t-butyloxycarbonyl (Boc), ethyloxycarbonyl, methyloxycarbonyl, trichloroethyloxycarbonyl, allyloxycarbonyl (Alloc), benzyloxocarbonyl (Cbz), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See, for example, March's Advanced Organic Chemistry: Reactions, Mechanisms, and Structure, M. B. Smith and J. March, 7^{th} Edition, John Wiley & Sons, 2013, Comprehensive Organic Transformations, R. C. Larock, 3^{rd} Edition, John Wiley & Sons, 2018, the entirety of each of which is incorporated herein by reference. Such interconversions may require one or more of the aforementioned techniques, and certain methods for synthesizing compounds of the invention are described below.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. Such groups and transformations are described in detail in March's Advanced Organic Chemistry: Reactions, Mechanisms, and Structure, M. B. Smith and J. March, 7^{th} Edition, John Wiley & Sons, 2013, Comprehensive Organic Transformations, R. C. Larock, 3^{rd} Edition, John Wiley & Sons, 2018, and Protective Groups in Organic Synthesis, P. G. M. Wuts, 5^{th} edition, John Wiley & Sons, 2014, the entirety of each of which is hereby incorporated herein by reference. Such interconversions may require one or more of the aforementioned techniques, and certain methods for synthesizing compounds of the invention are described below in the Exemplification and Figures.

### 4. Uses, Formulation and Administration

### Pharmaceutically Acceptable Compositions

In one aspect, the disclosure provides a composition comprising a compound of this invention or a pharmaceutically acceptable derivative thereof and a pharmaceutically acceptable carrier, adjuvant, or vehicle. The amount of compound in compositions of this invention is such that is effective to measurably modulate (e.g., inhibit or degrade) a target RNA transcript, or an isoform, mutant, or fragment thereof, in a biological sample or in a patient. In certain embodiments, the amount of compound in compositions of this invention is such that is effective to measurably inhibit or modulate a target RNA transcript, in a biological sample or in a patient. In certain embodiments, a composition of this invention is formulated for administration to a patient in need of such composition. In some embodiments, a composition of this invention is formulated for oral administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N. Y.; Fingl et al., The Pharmacological Basis of Therapeutics (1975), and Remington's Pharmaceutical Sciences, Mack Publishing Co., Easton, PA, 18^{th} edition (1990); each of which is hereby incorporated by reference in its entirety. These texts can, of course, also be referred to in making or using an aspect of the disclosure. The disclosure also provides pharmaceutical compositions comprising a compound of the disclosure or pharmaceutically acceptable salts thereof, and one or more other therapeutic agents disclosed herein, mixed with pharmaceutically suitable carriers or excipient(s) at doses to treat or prevent a disease or condition as described herein. The pharmaceutical compositions of the disclosure can also be administered in combination with other therapeutic agents or therapeutic modalities simultaneously, sequentially, or in alternation.

Mixtures of compositions of the disclosure can also be administered to the patient as a simple mixture or in suitable formulated pharmaceutical compositions. For example, some aspects of the disclosure relate to a pharmaceutical composition comprising a therapeutically effective dose of a compound of the disclosure, or a pharmaceutically acceptable salt, hydrate, enantiomer or stereoisomer thereof; one or more other therapeutic agents, and a pharmaceutically acceptable diluent or carrier.

A “pharmaceutical composition” is a formulation containing the compounds of the disclosure in a form suitable for administration to a subject. A compound of the disclosure and one or more other therapeutic agents described herein each can be formulated individually or in multiple pharmaceutical compositions in any combinations of the active ingredients.

Accordingly, one or more administration routes can be properly elected based on the dosage form of each pharmaceutical composition. Alternatively, a compound of the disclosure and one or more other therapeutic agents described herein can be formulated as one pharmaceutical composition.

### Uses of Compounds and Pharmaceutically Acceptable Compositions

Compounds and compositions described herein are generally useful for the modulation of a target RNA transcript to treat an RNA-mediated disease or condition. It should be appreciated that RNA-mediated diseases include all protein mediated diseases and conditions.

The activity of a compound utilized in this invention to modulate (e.g. degrade) a target RNA transcript may be assayed in vitro, in vivo, ex vivo, or in a cell line. In vitro assays include assays that determine modulation of the target RNA transcript. Alternate in vitro assays quantitate the ability of the compound to bind to the target RNA transcript. Detailed conditions for assaying a compound utilized in this invention to modulate a target RNA transcript are set forth in the Examples below.

The term “patient” or “subject,” as used herein, means an animal, preferably a mammal, and most preferably a human.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The present disclosure provides treatment modalities, methods, strategies, compositions, combinations, and dosage forms for the treatment of RNA-mediated diseases, disorders, and conditions.

Provided compounds are modulators of a target RNA transcript and are therefore useful for treating one or more disorders associated with or affected by (e.g., downstream of) the target RNA transcript. Thus, in certain embodiments, the present invention provides a method for treating an RNA-mediated disorder comprising the step of administering to a patient in need thereof a compound of the present invention, or pharmaceutically acceptable composition thereof.

In one aspect, the disclosure provides selective modulators of a target RNA transcript. For example, in some embodiments, the selective modulator (e.g., inhibitor or antagonist) has an IC₅₀ for a target RNA transcript that is at least 40 percent lower than the IC₅₀ for a non-target RNA transcript. In some embodiments, the selective modulator (e.g., inhibitor or antagonist) has an IC₅₀ for the target RNA transcript that is at least 50 percent lower than the IC₅₀ for the non-target RNA transcript. In some embodiments, the selective modulator (e.g., inhibitor or antagonist) has an IC₅₀ for the target RNA transcript that is at least 60, 70, 80, 90, or 95 percent lower than the IC₅₀ for the non-target RNA transcript. In some embodiments, the selective modulator (e.g., antagonist or inhibitor) of a target RNA transcript exerts essentially no inhibitory effect on a non-target RNA transcript.

In some embodiments, the selective modulator (e.g., inhibitor or antagonist) modulates the activity of a target RNA transcript at least 2-fold more efficiently than a non-target RNA transcript. In some embodiments, the selective modulator (e.g., inhibitor or antagonist) modulates the activity of a target RNA transcript at least 5-fold more efficiently than a non-target RNA transcript. In some embodiments, the selective modulator (e.g., inhibitor or antagonist) modulates the activity of a target RNA transcript at least 10-, 20-, 50-, 100-, 1000-, 10000-, or 100000-fold more efficiently than a non-target RNA transcript.

Disclosed compounds may be used to treat a variety of diseases, disorders, and conditions. In some embodiments, the present invention provides a method for treating one or more diseases, disorders, and conditions wherein the disorder, disease, or condition includes, but is not limited to, a cellular proliferative disorder.

### Cellular Proliferative Disorders

In one aspect, the present invention provides methods and compositions for the diagnosis and prognosis of cellular proliferative disorders (e.g., cancer) and the treatment of these disorders by modulating (e.g. degrading) a target RNA transcript. Cellular proliferative disorders described herein include, e.g., cancer, obesity, and proliferation-dependent diseases. Such disorders may be diagnosed using methods known in the art.

In one aspect, the present invention provides methods and compositions for the treatment of cancer by modulating (e.g. degrading) a target RNA transcript. In some embodiments, the cancer is driven or characterized by the overexpression of a protein (e.g. an oncogenic protein) and the cancer is treated by modulating (e.g. degrading) a target RNA transcript that corresponds to the overexpressed protein.

In one aspect, the present invention provides methods and compositions for the treatment of cancer. Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). In some embodiments, the cancer is melanoma or breast cancer.

Cancers includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkin's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the present invention provides a method for treating a tumor in a patient in need thereof, comprising administering to the patient any of the compounds, salts or pharmaceutical compositions described herein. In some embodiments, the tumor comprises any of the cancers described herein. In some embodiments, the tumor comprises melanoma cancer. In some embodiments, the tumor comprises breast cancer. In some embodiments, the tumor comprises lung cancer. In some embodiments the tumor comprises small cell lung cancer (SCLC). In some embodiments, the tumor comprises non-small cell lung cancer (NSCLC).

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplasia syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropuimonary biastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

In some embodiments, the disease, disorder, or condition is a cell proliferative disorder of the hematologic system. A “cell proliferative disorder of the hematologic system” is a cell proliferative disorder involving cells of the hematologic system. A cell proliferative disorder of the hematologic system includes lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system includes hyperplasia, dysplasia, and metaplasia of cells of the hematologic system. In some embodiments, the cancer is selected from a hematologic cancer disclosed herein or a hematologic cell proliferative disorder disclosed herein. Hematologic cancers include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms, and mast cell neoplasms.

In some embodiments, treating cancer results in a reduction in tumor volume. In some embodiments, after treatment, tumor volume is reduced by 5% or greater relative to its size prior to treatment; tumor volume is reduced by 10% or greater; 20% or greater; 30% or greater; 40% or greater; 50% or greater; or 75% or greater. Tumor volume may be measured by any reproducible means of measurement.

In some embodiments, treating cancer results in a decrease in number of tumors. In some embodiments, after treatment, tumor number is reduced by 5% or greater relative to number prior to treatment; or tumor number is reduced by 10% or greater; 20% or greater; 30% or greater; 40% or greater; 50% or greater; or by greater than 75%. Number of tumors may be measured by any reproducible means of measurement. The number of tumors may be measured by counting tumors visible to the naked eye or at a specified magnification. For example, the specified magnification is selected from 2×, 3×, 4×, 5×, 10×, or 50×.

In some embodiments, treating cancer results in a decrease in number of metastatic lesions in other tissues or organs distant from the primary tumor site. In some embodiments, after treatment, the number of metastatic lesions is reduced by 5% or greater relative to number prior to treatment; or reduced by 10% or greater; 20% or greater; 30% or greater; 40% or greater; 50% or greater; or reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

In some embodiments, treating cancer results in an increase in average survival time of a population of treated subjects in comparison to a population receiving carrier alone. In some embodiments, the average survival time is increased by more than 30 days; or by more than 60 days; more than 90 days; or by more than 120 days.

In some embodiments, treating cancer results in an increase in average survival time of a population of treated subjects in comparison to a population of untreated subjects. In some embodiments, the average survival time is increased by more than 30 days; or by more than 60 days; more than 90 days; or by more than 120 days.

In some embodiments, treating cancer can result in increase in average survival time of a population of treated subjects in comparison to a population receiving monotherapy with a drug that is not a compound of the present disclosure, or a pharmaceutically acceptable salt, solvate, analog or derivative thereof. In some embodiments, the average survival time is increased by more than 30 days; or more than 60 days; more than 90 days; or by more than 120 days.

An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

In some embodiments, treating cancer results in a decrease in the mortality rate of a population of treated subjects in comparison to a population receiving carrier alone. In some embodiments, treating cancer results in a decrease in the mortality rate of a population of treated subjects in comparison to an untreated population. In some embodiments, treating cancer results in a decrease in the mortality rate of a population of treated subjects in comparison to a population receiving monotherapy with a drug that is not a compound of the present disclosure, or a pharmaceutically acceptable salt, solvate, analog or derivative thereof. In some embodiments, the mortality rate is decreased by more than 2%; more than 5%; more than 10%; or by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

In some embodiments, treating cancer results in a decrease in tumor growth rate. In some embodiments, after treatment, tumor growth rate is reduced by at least 5% relative to number prior to treatment; or reduced by at least 10%; at least 20%; at least 30%; at least 40%; at least 50%; at least 50%; or at least 75%. Tumor growth rate may be measured by any reproducible means of measurement. Tumor growth rate can be measured according to a change in tumor diameter per unit time.

In some embodiments, treating cancer can result in a decrease in tumor regrowth. In some embodiments, after treatment, tumor regrowth is less than 5%; less than 10%; less than 20%; less than 30%; less than 40%; less than 50%; less than 50%; or less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

In some embodiments, treating or preventing a cell proliferative disorder results in a reduction in the rate of cellular proliferation. In some embodiments, after treatment, the rate of cellular proliferation is reduced by at least 5%; at least 10%; at least 20%; at least 30%; at least 40%; at least 50%; at least 50%; or at least 75%. The rate of cellular proliferation may be measured by any reproducible means of measurement. The rate of cellular proliferation is measured, for example, by measuring the number of dividing cells in a tissue sample per unit time.

In some embodiments, treating or preventing a cell proliferative disorder results in a reduction in the proportion of proliferating cells. In some embodiments, after treatment, the proportion of proliferating cells is reduced by at least 5%; at least 10%; at least 20%; at least 30%; at least 40%; at least 50%; at least 50%; or at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

In some embodiments, treating or preventing a cell proliferative disorder results in a decrease in size of an area or zone of cellular proliferation. In some embodiments, after treatment, size of an area or zone of cellular proliferation is reduced by at least 5% relative to its size prior to treatment; at least 10%; at least 20%; at least 30%; at least 40%; at least 50%; at least 50%; or at least 75%. Size of an area or zone of cellular proliferation may be measured by any reproducible means of measurement. The size of an area or zone of cellular proliferation may be measured as a diameter or width of an area or zone of cellular proliferation.

In some embodiments, treating or preventing a cell proliferative disorder results in a decrease in the number or proportion of cells having an abnormal appearance or morphology. In some embodiments, after treatment, the number of cells having an abnormal morphology is reduced by at least 5% relative to its size prior to treatment; at least 10%; at least 20%; at least 30%; at least 40%; at least 50%; at least 50%; or reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

In some embodiments, a subject in need thereof has refractory or resistant cancer. “Refractory or resistant cancer” means cancer that does not respond to an established line of treatment. In some embodiments, the cancer is resistant at the beginning of treatment or becomes resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In some embodiments, the prior therapy is monotherapy. In some embodiments, the prior therapy is combination therapy.

In some embodiments, a subject in need thereof has a secondary cancer as a result of a previous therapy. “Secondary cancer” means cancer that arises due to or as a result from previous carcinogenic therapies, such as chemotherapy.

As used herein, the term “responsiveness” is interchangeable with terms “responsive,” “sensitive,” and “sensitivity,” and it is meant that a subject is showing a therapeutic response when administered a composition of the disclosure, e.g., tumor cells or tumor tissues of the subject undergo apoptosis and/or necrosis, and/or display reduced growing, dividing, or proliferation. In some embodiments, a “response” also means that a subject will have or has a higher probability, relative to the population at large, of showing therapeutic responses when administered a disclosed compound, e.g., tumor cells or tumor tissues of the subject undergo apoptosis and/or necrosis, and/or display reduced growing, dividing, or proliferation.

As used herein, “sample” means any biological sample derived from the subject and includes, but is not limited to, cells, tissue samples, body fluids (including, but not limited to, mucus, blood, plasma, serum, urine, saliva, and semen), tumor cells, and tumor tissues.

In some embodiments, the sample is selected from bone marrow, peripheral blood cells, blood, plasma, and serum. Samples can be provided by the subject under treatment or testing. Alternatively, samples can be obtained by the physician according to routine practice in the art.

As used herein, a “normal cell” is a cell that cannot be classified as part of a “cell proliferative disorder.” A normal cell lacks unregulated or abnormal growth, or both, that can lead to the development of an unwanted condition or disease. Typically, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

In one aspect, the present invention provides methods and compositions for the diagnosis and prognosis of cellular proliferative disorders that are not commonly characterized as cancer, and the treatment of these disorders by modulating (e.g. degrading) a target RNA transcript. Other proliferative diseases include, e.g., obesity, benign prostatic hyperplasia, psoriasis, abnormal keratinization, lymphoproliferative disorders (e.g., a disorder in which there is abnormal proliferation of cells of the lymphatic system), chronic rheumatoid arthritis, arteriosclerosis, restenosis, and diabetic retinopathy. Proliferative diseases that are hereby incorporated by reference include those described in U.S. Pat. Nos. 5,639,600 and 7,087,648.

As used herein, the term “selectively” means tending to occur at a higher frequency in one population than in another population. The compared populations can be cell populations. In some embodiments, a compound of the disclosure, or a pharmaceutically acceptable salt or solvate thereof, acts selectively on a cancer or precancerous cell but not on a normal cell. In some embodiments, a disclosed compound, or a pharmaceutically acceptable salt or solvate thereof, acts selectively to modulate one molecular target but does not significantly modulate another molecular target. The present invention also provides a method for selectively inhibiting the activity of a target RNA.

In some embodiments, treating cancer or a cell proliferative disorder results in cell death. In some embodiments, cell death results in a decrease of at least 10% in number of cells in a population. In some embodiments, cell death means a decrease of at least 20%; at least 30%; at least 40%; at least 50%; or at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA, 100 (5): 2674-8, 2003. In some aspects, cell death occurs by apoptosis.

In some embodiments, an effective amount of a disclosed compound, or a pharmaceutically acceptable salt or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In some aspects, cell death occurs by apoptosis.

In some embodiments, the present invention provides a method of treating or preventing cancer by administering a an effective amount of a disclosed compound, or a pharmaceutically acceptable salt or solvate thereof, to a subject in need thereof, wherein administration of the compound, or a pharmaceutically acceptable salt or solvate thereof, results in one or more of the following: prevention of cancer cell proliferation by accumulation of cells in one or more phases of the cell cycle (e.g. Gl, Gl/S, G2/M), or induction of cell senescence, or promotion of tumor cell differentiation; promotion of cell death in cancer cells via cytotoxicity, necrosis or apoptosis, without a significant amount of cell death in normal cells, antitumor activity in animals with a therapeutic index of at least 2. As used herein, “therapeutic index” is the maximum tolerated dose divided by the efficacious dose.

### Formulations and Routes of Administration

The compounds and compositions, according to a method of the present invention, may be administered using any amount and any route of administration effective for treating or lessening the severity of a cancer or other disease, disorder, or condition disclosed herein. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “unit dosage form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient” or “subject,” as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of modulating the activity of a target RNA transcript in a biological sample comprising the step of contacting said biological sample with a compound of this invention, or a composition comprising said compound.

According to another embodiment, the invention relates to a method of modulating the activity of a target RNA transcript in a biological sample comprising the step of contacting said biological sample with a compound of this invention, or a composition comprising said compound. In certain embodiments, the invention relates to a method of irreversibly inhibiting the activity of a target RNA transcript in a biological sample comprising the step of contacting the biological sample with a compound of this invention, or a composition comprising the compound.

The term “biological sample,” as used herein, includes, without limitation, cell cultures or extracts thereof; biopsied material obtained from a mammal or extracts thereof; and blood, saliva, urine, feces, semen, tears, cerebrospinal fluid, or other body fluids or extracts thereof.

Another embodiment of the present invention relates to a method of modulating the activity of a target RNA transcript in a patient comprising the step of administering to said patient a compound of the present invention, or a composition comprising said compound.

According to another embodiment, the invention relates to a method of inhibiting the activity of a target RNA transcript in a patient comprising the step of administering to said patient a compound of the present invention, or a composition comprising said compound. According to certain embodiments, the invention relates to a method of irreversibly inhibiting the activity of a target RNA transcript in a patient comprising the step of administering to said patient a compound of the present invention, or a composition comprising said compound. In other embodiments, the present invention provides a method for treating a disorder mediated by a target RNA transcript in a patient in need thereof, comprising the step of administering to said patient a compound according to the present invention or pharmaceutically acceptable composition thereof. Such disorders are described in detail herein.

### EXEMPLIFICATION

As depicted in the Examples below, exemplary compounds are prepared according to the following general procedures and used in biological assays and other procedures described generally herein. It will be appreciated that, although the general methods depict the synthesis of certain compounds of the present invention, the following general methods, and other methods known to one of ordinary skill in the art, can be applied to all compounds and subclasses and species of each of these compounds, as described herein. Similarly, assays and other analyses can be adapted according to the knowledge of one of ordinary skill in the art.

### Example 1: Genetic Tethering Assays

### Genetic Tethering General Description

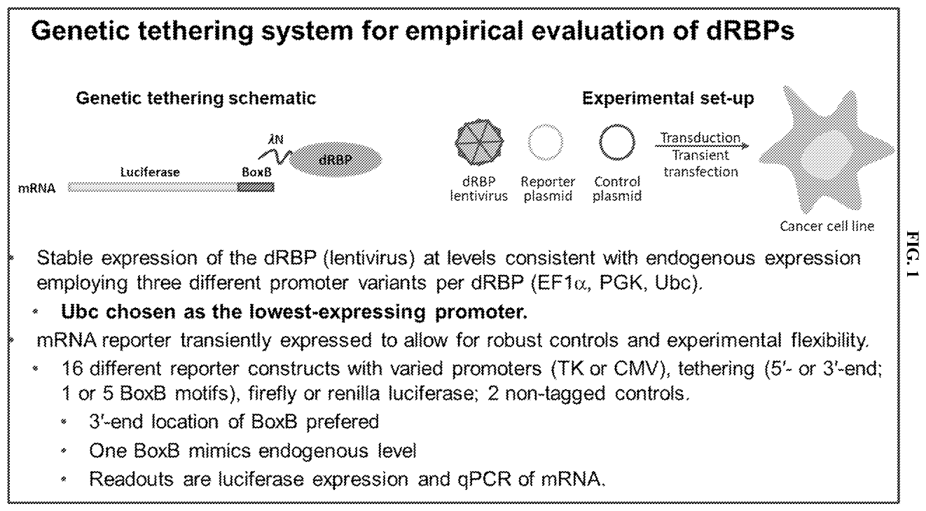

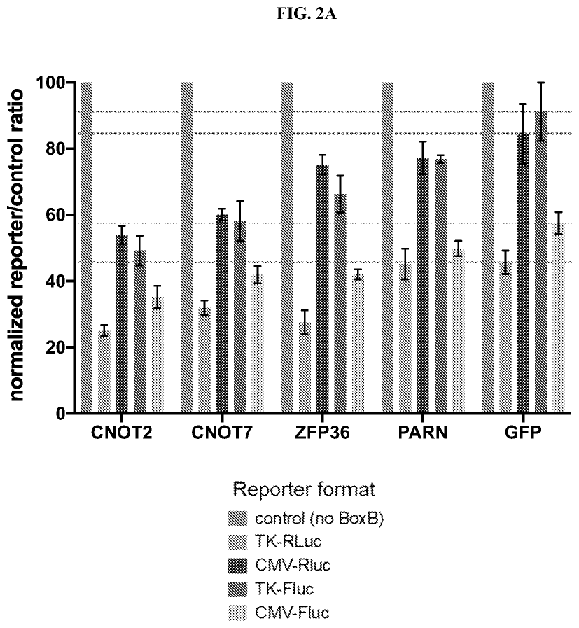

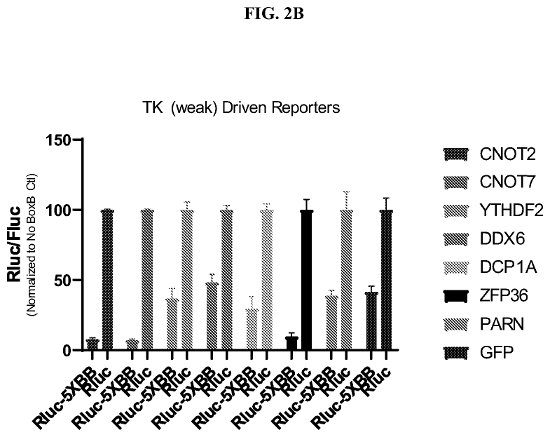

NCI-H1299, a human non-small cell lung cancer cell line (ATCC) were engineered to stably express various λN-tagged RNA-binding proteins (RBPs) via lentivirus. Cells were then transfected using Lipofectamine 3000 (Thermo Fisher) with two plasmids: one encoding a reporter luciferase containing a BoxB site in either the 3′ untranslated region (UTR) or the 5′ UTR and one normalizer plasmid encoding an alternative luciferase without any BoxB sites for tethering. After 24 hours, expression of both luciferases was measured using commercially available kits (Promega). Data are expressed as a ratio of reporter expression (measured in relative light units or RLUs) to normalizer expression. An overview of the assay is provided in FIG. 1.

### Transfection

NCI-H1299 cells were seeded on Day 0 in 96-well plates at 5000 cells per well in Roswell Park Memorial Institute (RPMI) 1640 medium containing 10% Fetal Bovine Serum and incubated overnight at 37 degrees Celsius under 5% carbon dioxide (CO₂). On day 1, transfection mixes were prepared with reporter and normalizer constructs containing BoxB sites for genetic tethering or no BoxB sites, respectively. Transfections were performed according to manufacturer instructions for Lipofectamine 3000 (Thermo Fisher). After approximately 24 hours, luciferase activity was measured using the Dual Luciferase Reporter Assay System (Promega).

### Lentiviral Production and Cell Transduction

Lentivirus encoding λN-tagged RNA-binding proteins (RBPs) was prepared using the ViraPower Lentiviral Packaging Mix (Thermo Fisher) according to manufacturer protocols. On Day 0, cells were transduced via centrifugation with polybrene for 1 hour at 1000 relative centrifugal force (RCF). After centrifugation, cells were kept at 37 degrees Celsius, 5% CO₂ overnight. Next, cells were expanded in standard tissue culture flasks under selective pressure of puromycin. Cells were passaged in appropriate cell culture medium for downstream applications.

### Quantification of RNA-Binding Protein (RBP) Expression

Protein expression of RBPs were measured using an automated in-capillary electrophoresis assay according to manufacturer protocols (ProteinSimple). Appropriate antibodies targeting either the RBP or the hemagglutinin (HA) tag were purchased from commercial vendors (i.e., Sigma Aldrich).

Quantitative Polymerase Chain Reaction (qPCR) Assay Development and Optimization

The qPCR assay reagents were ordered based on manufacturer's specification (Applied Biosystems). Standard software, such as Primer BLAST were used to generate optimal qPCR amplification primers and probes specific to Firefly and Renilla Luciferase complementary deoxyribonucleic acid (cDNA). The PCR primers were ordered from Integrated DNA Technologies (IDT). The gene specific probes were generated with 5-carboxyfluorescein (5′ FAM), nonfluorescent quencher (NFQ) and the minor groove binder (MGB).

qPCR assays for cDNA quantification and primer validation were conducted using Taqman Fast Advanced Master Mix (Life Technologies). Gene quantification and plots were conducted using Excel and Prism software, respectively.

### Vector Design and Synthesis

The desired RBP and reporter gene expressing constructs were generated using Geneious Primer software. The preferred RBP isoform and luciferase sequences were obtained from the National Center for Biotechnology Information (NCBI).

Vector cloning and transfection grade plasmid preparations were conducted at Genscript. The base mammalian expression vectors for cloning were obtained from the vector database at Arrakis Therapeutics.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula A: 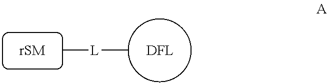
or a pharmaceutically acceptable salt thereof, wherein:
rSM is an RNA-binding small molecule that binds to a target RNA transcript;
DFL is a Decay Factor-recruiting Ligand; and
L is a bivalent linker group that covalently connects the rSM to the DFL;
wherein the DFL binds to or recruits a decay factor.

2. The compound of claim 1, wherein the decay factor is an RNA-binding protein (RBP) and wherein binding of the DFL to the RBP leads to modulation of the target RNA transcript in vivo.

3. The compound of claim 2, wherein modulation of the target RNA transcript in vivo is degradation of the target RNA transcript.

4. The compound of claim 2, wherein the DFL binds the RBP without abrogating the enzymatic activity of the RBP and/or the ability of the RBP to be part of a multi-component complex.

5. The compound of claim 2, wherein the RBP is an endonuclease, an exonuclease, a deadenylase, or a decapping protein; or wherein the RBP is part of a multi-component complex that has endonuclease, exonuclease, deadenylase, or decapping activity; or wherein the RBP destabilizes the target RNA transcript's 3-dimensional structure in a manner that makes it more prone to degradation.

6. (canceled)

7. The compound of claim 2, wherein the RBP has enzymatic activity, or is part of a multi-component complex that has enzymatic activity, at endogenous levels in vivo sufficient to measurably modulate the target RNA transcript or destabilize its 3-dimensional structure in a manner that makes it more prone to degradation.

8.-10. (canceled)

11. The compound of claim 2, wherein the RBP is part of the CCR4-NOT (Carbon Catabolite Repression-Negative On TATA-less) complex.

12.-19. (canceled)

20. The compound of claim 1, wherein L is a covalent bond or a bivalent, saturated or unsaturated, straight or branched, optionally substituted C₁₋₅₀ hydrocarbon chain, wherein 0, 1, 2, 3, 4, 5, 6, 7, 8, 9, or 10 methylene units of L are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(NR₂)—, an amino acid, 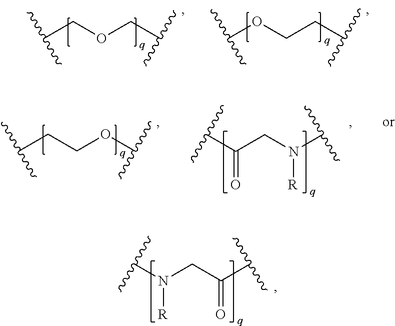
wherein:
each -Cy²- is independently an optionally substituted bivalent ring selected from phenylene, an 8-12 membered bicyclic arylene, a 3-8 membered saturated or partially unsaturated carbocyclylene, an 8-12 membered bicyclic saturated or partially unsaturated carbocyclylene, a 3-8 membered saturated or partially unsaturated heterocyclylene having 1-3 heteroatoms independently selected from nitrogen, oxygen, and sulfur, an 8-12 membered bicyclic saturated or partially unsaturated heterocyclylene having 1-5 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur; and
each q is independently 1, 2, or 3.

21.-43. (canceled)

44. A pharmaceutical composition comprising the compound of claim 1, or a pharmaceutically acceptable salt thereof, and a pharmaceutically acceptable carrier.

45. A method of modifying the amount of a protein in a cell, the method comprising administering a compound or composition that acts on a target RNA transcript or a precursor, isoform, fragment, or mutant thereof, in an amount sufficient to modify the amount of the protein in the cell.

46. The method of claim 45, wherein modifying the amount of a protein in a cell is reducing the amount of protein in the cell.

47. The method of claim 45, comprising administering a compound of Formula A: 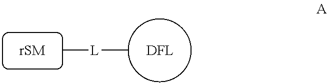
or a pharmaceutically acceptable salt thereof, wherein:
rSM is an RNA-binding small molecule that binds to a target RNA transcript;
DFL is a Decay Factor-recruiting Ligand; and
L is a bivalent linker group that covalently connects the rSM to the DFL;
wherein the DFL binds to or recruits a decay factor.

48.-50. (canceled)

51. A method of treating a disease, comprising administering to a subject in need thereof a compound of Formula A: 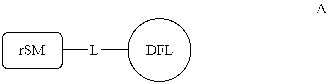
or a pharmaceutically acceptable salt thereof, wherein:
rSM is an RNA-binding small molecule that binds to a target RNA transcript;
DFL is a Decay Factor-recruiting Ligand; and
L is a bivalent linker group that covalently connects the rSM to the DFL;
wherein the DFL binds to or recruits a decay factor.

52. The method of claim 51, wherein the disease is characterized by an aberrant level of a protein in a cell.

53. (canceled)

54. The method of claim 52, wherein the disease is a cancer.

55.-59. (canceled)
